(19) United States
(12) Patent Application Publication
(10) Pub. No.: US 2012/0070430 A1
(43) Pub. Date: Mar. 22, 2012

(54) IDENTIFICATION OF TUMOR-ASSOCIATED MARKERS FOR DIAGNOSIS AND THERAPY

(76) Inventors: Ugur Sahin, Mainz (DE); Ozlem Tureci, Mainz (DE); Michael Koslowski, Frankfurt (DE)

(21) Appl. No.: 13/266,368

(22) PCT Filed: Apr. 22, 2010
(86) PCT No.: PCT/EP10/02490

§ 371 Date: Oct. 26, 2011

Related U.S. Application Data
(60) Provisional application No. 61/173,871, filed on Apr. 29, 2009.

(30) Foreign Application Priority Data
Apr. 29, 2009 (EP) 09005931.2

(51) Int. Cl. A61K 39/395; C07K 16/18; C07K 14/47; C12N 5/10; C12N 1/21; A61P 35/00; C07H 21/00; A61K 39/00; C12Q 1/68; G01N 33/53; A61K 35/12; C07H 21/04; C12N 1/19

(52) U.S. Cl. 424/133.1; 536/24.5; 530/389.7; 530/350; 435/325; 435/252.33; 435/254.2; 435/348; 435/353; 435/354; 435/358; 435/365; 435/363; 435/366; 536/23.5; 435/372.1; 530/388.85; 530/387.3; 424/184.1; 435/6.14; 435/7.1; 436/501; 424/174.1; 424/156.1; 424/93.21

(57) ABSTRACT

The invention relates to genetic products the expression of which is associated with cancer diseases. The invention also relates to the therapy and diagnosis of diseases in which the genetic products are expressed or aberrantly expressed, in particular cancer diseases.

The invention relates to nucleic acids and encoded polypeptides which are expressed in cancers. The invention also relates to agents which bind the polypeptides. The nucleic acids, polypeptides coded for by such nucleic acids and peptides derived therefrom, as well as related antibodies and cytolytic T lymphocytes, are useful, inter alia, in diagnostic and therapeutic contexts.

Despite interdisciplinary approaches and exhaustive use of classical therapeutic procedures, cancers are still among the leading causes of death.

More recent therapeutic concepts in cancer therapy aim at incorporating the patient's immune system into the overall therapeutic concept by using recombinant tumor vaccines and other specific measures such as antibody therapy. A prerequisite for the success of such a strategy is the recognition of tumor-specific or tumor-associated antigens or epitopes by the patient's immune system whose effector functions are to be interventionally enhanced.

Tumor cells biologically differ substantially from their nonmalignant cells of origin. These differences are due to genetic alterations acquired during tumor development and result, inter alia, also in the formation of qualitatively or quantitatively altered molecular structures in the cancer cells. Tumor-associated structures of this kind which are recognized by the specific immune system of the tumor-harboring host are referred to as tumor-associated antigens.

The specific recognition of tumor-associated antigens involves cellular and humoral mechanisms which are two functionally interconnected units: CD4⁺ and CD8⁺ T lymphocytes recognize the processed antigens presented on the molecules of the MHC (major histocompatibility complex) classes II and I, respectively, while B lymphocytes produce circulating antibody molecules which bind directly to unprocessed antigens. The potential clinical-therapeutical importance of tumor-associated antigens results from the fact that the recognition of antigens on neoplastic cells by the immune system leads to the initiation of cytotoxic effector mechanisms and, in the presence of T helper cells, can cause elimination of the cancer cells (Pardoll, Nat. Med. 4:525-31, 1998).

Antibody based cancer therapies have been successfully introduced into the clinic and have emerged as the most promising therapeutics in oncology over the last decade. Eight antibodies have been approved for treatment of neoplastic diseases, most of them, however in lymphoma and leukemia (Adams G P, Weiner L M, Nat Biotechnol 23:1147-57, 2005).

One of the challenges to be mastered for the advent of the next generation of upgraded antibody-based cancer therapeutics is the selection of appropriate target molecules, which is the key for a favorable toxicity/efficacy profile.

The search for genes tightly silenced in the vast majority of healthy tissues moves into the focus of attention the intriguing observation that genes of the gametogenic and/or trophoblastic lineage are frequently ectopically activated and robustly expressed in human cancer. Based on phenotypical similarities between germ cells, pregnancy trophoblast and cancer cells, John Beard proposed as much as 100 years ago a “trophoblastic theory of cancer” (Beard J, Lancet 1:1758-63, 1902; Gurchot C, Oncology 31:310-3, 1975). The discovery of the sporadic production of chorionic gonadotropin, alpha-fetoprotein, CEA and other trophoblastic hormones by cancer cells provided the first molecules shared between neoplastic and trophoblastic cells (Acevedo H F et al., Cancer 76:1467-75, 1995; Dirnhofer S et al., Hum Pathol 29:377-82, 1998; Gurchot C, Oncology 31:310-3, 1975; Iles R K, Chard T, J Urol 145:453-8, 1991; Laurence D J, Neville A M, Br J Cancer 26:335-55, 1972). The concept was reignited by the inauguration of the steadily growing so-called cancer/germline (CG) class of genes, which represents more than 100 members, each expressed in a variety of tumor types. The observation that entire trophoblastic and gametogenic programs escape transcriptional silencing and are ectopically activated in cancer cells (Koslowski M et al., Cancer Res 64:5988-93, 2004; Simpson A J et al., Nat Rev Cancer 5:615-25, 2005) indicates that within this class of genes with exquisitely selective tissue distribution, appropriate targets for mAB therapy may be found.

It was the object of the present invention to provide target structures for a diagnosis and therapy of cancers.

This object is achieved by the subject matter of the claims.

According to the invention, placenta-specific genes are identified which are selectively or aberrantly expressed in tumor cells and thus, provide target structures for therapeutic and diagnostic approaches.

The nucleic acids identified according to the invention to be selectively or aberrantly expressed in tumor cells are selected from the group consisting of (a) a nucleic acid which comprises a nucleic acid sequence selected from the group consisting of SEQ ID NOs: 1-540, 541, 545, 549, 553, 557, 560, 563, 566, 570, 574, 577, 580, 583, 587, 591, 595, 599, 602, 606, 610, 613, 617, 620, 624, 634, 638, 642, 646, 649, 653, 656, 660, 664, 668, 671, 675, 679, 682, and 686 of the sequence listing, a part or derivative thereof, (b) a nucleic acid which hybridizes with the nucleic acid of (a) under stringent conditions, (c) a nucleic acid which is degenerate with respect to the nucleic acid of (a) or (b), and (d) a nucleic acid which is complementary to the nucleic acid of (a), (b) or (c). These nucleic acids are also termed “tumor-associated nucleic acids” herein.

In another aspect, the invention relates to antigens encoded by the tumor-associated nucleic acids identified according to the invention. Accordingly, the tumor-associated antigens identified according to the invention have an amino acid sequence encoded by a nucleic acid which is selected from the group consisting of (a) a nucleic acid which comprises a nucleic acid sequence selected from the group consisting of SEQ ID NOs: 1-540, 541, 545, 549, 553, 557, 560, 563, 566, 570, 574, 577, 580, 583, 587, 591, 595, 599, 602, 606, 610, 613, 617, 620, 624, 634, 638, 642, 646, 649, 653, 656, 660, 664, 668, 671, 675, 679, 682, and 686 of the sequence listing, a part or derivative thereof, (b) a nucleic acid which hybridizes with the nucleic acid of (a) under stringent conditions, (c) a nucleic acid which is degenerate with respect to the nucleic acid of (a) or (b), and (d) a nucleic acid which is complementary to the nucleic acid of (a), (b) or (c). In a preferred embodiment, the tumor-associated antigens identified according to the invention comprises an amino acid sequence selected from the group consisting of SEQ ID NOs: 542, 546, 550, 554, 567, 571, 584, 588, 592, 596, 603, 607, 614, 621, 625, 635, 639, 643, 650, 657, 661, 665, 672, 676, and 683 of the sequence listing, a part or derivative thereof.

If according to the invention reference is made to nucleic acids comprising certain nucleic acid sequences or tumor-associated antigens comprising certain amino acid sequences this also includes embodiments wherein the nucleic acids or tumor-associated antigens consist of these certain nucleic acid sequences or amino acid sequences, respectively.

The present invention generally embraces the treatment and/or diagnosis by targeting tumor-associated nucleic acids or tumor-associated antigens identified according to the invention. This provides for the selective detection of cells and/or eradication of cells that express such tumor-associated nucleic acids and/or tumor-associated antigens thereby minimizing adverse effects to normal cells not expressing such tumor-associated nucleic acids and/or tumor-associated antigens. Thus, preferred diseases for a therapy or diagnosis are those in which one or more of the tumor-associated nucleic acids and/or tumor-associated antigens identified according to the invention are expressed such as tumor diseases, in particular cancer diseases such as those described herein.

The present invention generally involves the use of tumor-associated nucleic acids and tumor-associated antigens identified according to the invention or of parts or derivatives thereof, of nucleic acids directed against said tumor-associated nucleic acids, of antibodies or T cells directed against the tumor-associated antigens identified according to the invention or parts or derivatives thereof and/or of host cells expressing the tumor-associated antigens identified according to the invention or parts or derivatives thereof for therapy, prophylaxis, diagnosis and/or monitoring of neoplastic diseases such as tumor diseases, in particular cancer diseases such as those described herein.

This may also involve the use of a combination of two or more of these nucleic acids, antigens, antibodies, T cells and/or host cells.

In those embodiments of the invention relating to the use of antibodies directed against the tumor-associated antigens identified according to the invention or parts or derivatives thereof also T cell receptors directed against the tumor-associated antigens identified according to the invention or parts or derivatives thereof, optionally in a complex with MHC molecules, may be used.

Especially suitable for therapy, prophylaxis, diagnosis and/or monitoring is a part of the tumor-associated antigens identified according to the invention which corresponds to the non-transmembrane portion, in particular the extracellular portion of the tumor-associated antigens or is comprised thereof. Therefore, according to the invention, a part of the tumor-associated antigens identified according to the invention which corresponds to the non-transmembrane portion, in particular the extracellular portion of the tumor-associated antigens or is comprised thereof, or a corresponding part of the nucleic acids coding for the tumor-associated antigens identified according to the invention is preferred for therapy, prophylaxis, diagnosis and/or monitoring. Similarly the use of antibodies is preferred which are directed against a part of the tumor-associated antigens identified according to the invention which corresponds to the non-transmembrane portion, in particular the extracellular portion of the tumor-associated antigens or is comprised thereof.

Preferred diseases for a therapy, prophylaxis, diagnosis and/or monitoring are those in which one or more of the tumor-associated nucleic acids identified according to the invention are selectively expressed or abnormally expressed. Particularly preferred diseases for a therapy, prophylaxis, diagnosis and/or monitoring are those in which one or more of the tumor-associated nucleic acids identified according to the invention and/or one or more of the tumor-associated antigens encoded thereby are selectively expressed or abnormally expressed.

In one aspect, the invention relates to a pharmaceutical composition comprising an agent which recognizes a tumor-associated antigen identified according to the invention or a nucleic acid coding for the tumor-associated antigen and which is preferably selective for cells which have expression or abnormal expression of a tumor-associated antigen identified according to the invention or a nucleic acid coding for the tumor-associated antigen.

In a further aspect, the invention relates to a pharmaceutical composition comprising an agent which (I) inhibits expression or activity of a tumor-associated antigen identified according to the invention, and/or (II) has tumor-inhibiting or tumor-destroying activity and is selective for cells expressing or abnormally expressing a tumor-associated nucleic acid and/or a tumor-associated antigen identified according to the invention, and/or (III) when administered, selectively increases the amount of complexes between an MHC molecule and a tumor-associated antigen identified according to the invention or a part thereof, such as a peptide epitope. In particular embodiments, said agent may cause induction of cell death, reduction in cell growth, damage to the cell membrane or secretion of cytokines and preferably have a tumor-inhibiting activity.

In one embodiment, an agent which inhibits expression or activity of a tumor-associated antigen is specific for a tumor-associated nucleic acid identified according to the invention. In another embodiment, an agent which inhibits expression or activity of a tumor-associated antigen is specific for a tumor-associated antigen identified according to the invention. According to the invention the phrase “inhibit expression and/or activity” includes a complete or essentially complete inhibition of expression and/or activity and a reduction in expression and/or activity. Preferably, said inhibition of expression of a tumor-associated antigen identified according to the invention may take place by inhibiting the production of or reducing the level of transcript, i.e. mRNA, coding for a tumor-associated antigen identified according to the invention, e.g. by inhibiting transcription or inducing degradation of transcript, and/or by inhibiting the production of tumor-associated antigen identified according to the invention, e.g. by inhibiting translation of transcript coding for a tumor-associated antigen identified according to the invention. Preferably, said inhibition of expression and/or activity of a tumor-associated antigen identified according to the present invention reduces tumor cell growth and/or induces tumor cell death and thus, has a tumor-inhibiting or tumor-destroying effect.

In a particular embodiment, the agent which inhibits expression of a tumor-associated antigen identified according to the invention is an inhibitory nucleic acid (e.g., anti-sense oligonucleotide, ribozyme, iRNA, siRNA or a DNA encoding the same) selectively hybridizing to and being specific for a tumor-associated nucleic acid identified according to the invention, thereby inhibiting (e.g., reducing) transcription and/or translation thereof.

Inhibitory nucleic acids include oligonucleotides having sequences in the antisense orientation relative to the target nucleic acids. Suitable inhibitory oligonucleotides typically vary in length from five to several hundred nucleotides, more typically about 20-70 nucleotides in length or shorter, even more typically about 10-30 nucleotides in length. These inhibitory oligonucleotides may be administered as free (naked) nucleic acids or in protected forms, e.g., encapsulated in liposomes. The use of liposomal or other protected forms may be advantageous as it may enhance in vivo stability and thus facilitate delivery to target sites.

Also, the target tumor-associated nucleic acid may be used to design ribozymes that target the cleavage of the corresponding mRNAs in tumor cells. Similarly, these ribozymes may be administered in free (naked) form or by the use of delivery systems that enhance stability and/or targeting, e.g., liposomes.

Also, the target tumor-associated nucleic acid may be used to design siRNAs that can inhibit (e.g., reduce) expression of the tumor-associated nucleic acid. The siRNAs may be administered in free (naked) form or by the use of delivery systems that enhance stability and/or targeting, e.g., liposomes. They may also be administered in the form of their precursors or encoding DNAs.

In a further embodiment, the agent which inhibits activity of a tumor-associated antigen identified according to the invention is an antibody that specifically binds to said tumor-associated antigen. Binding of the antibody to the tumor-associated antigen can interfere with the function of the tumor-associated antigen, e.g. by inhibiting binding activity or catalytic activity.

Also, the present invention in another aspect relates to therapies for treatment of tumor diseases involving the administration of a ligand of a target molecule, i.e. a tumor-associated nucleic acid or tumor-associated antigen identified according to the invention. In this respect, a nucleic acid may be administered that selectively hybridizes to the target nucleic acid or an antibody may be administered that specifically binds to a target antigen, attached to therapeutic effector moieties, e.g., radiolabels, cytotoxins, cytotoxic enzymes, and the like in order to selectively target and kill cells that express these targets, e.g. tumor cells.

In one embodiment of the above aspects, the agent is an antisense nucleic acid which hybridizes selectively with the nucleic acid coding for the tumor-associated antigen. In a further embodiment, the agent is a siRNA preferably comprising a sense RNA strand and an antisense RNA strand, wherein the sense and antisense RNA strands form an RNA duplex, and wherein the sense RNA strand comprises a nucleotide sequence substantially identical to a target sequence of about 19 to about 25 contiguous nucleotides in a nucleic acid coding for the tumor-associated antigen, preferably mRNA coding for the tumor-associated antigen. In a further embodiment, the agent is an antibody which binds selectively to the tumor-associated antigen, in particular a complement-activated or toxin conjugated antibody which binds selectively to the tumor-associated antigen. In a preferred embodiment, the antibody which binds selectively to the tumor-associated antigen is coupled to a therapeutically useful substance and/or recruits natural or artificial effector mechanisms to said cell expressing or abnormally expressing said tumor-associated antigen. In a further embodiment, the agent is a cytotoxic T lymphocyte which recognizes the tumor-associated antigen or a part thereof bound by an MHC molecule on a cell and lyses the cells labeled in this way. In a further embodiment, the agent is a T helper lymphocyte which recognizes the tumor-associated antigen or a part thereof bound by an MHC molecule on a cell and enhances effector functions of other cells specifically recognizing said tumor-associated antigen or a part thereof.

In a further embodiment, the agent comprises two or more agents which each recognize different tumor-associated antigens or different nucleic acids coding for tumor-associated antigens and/or inhibit expression or activity of different tumor-associated antigens, and/or have tumor-inhibiting or tumor-destroying activity and are selective for cells expressing or abnormally expressing different tumor-associated antigens, and/or when administered, selectively increase the amount of complexes between MHC molecules and different tumor-associated antigens or parts thereof, wherein at least one of said different tumor-associated antigens is a tumor-associated antigen identified according to the invention.

Preferably, a tumor-associated antigen selectively limited to tumors serves as a label for recruiting effector mechanisms to this specific location. In this aspect, the invention includes embodiments wherein the agent itself does not have an ability to inhibit activity of a tumor-associated antigen or a tumor-inhibiting or tumor-destroying activity but mediates such effect, in particular by recruiting effector mechanisms, in particular those having cell damaging potential, to a specific location, in particular a tumor or tumor cells.

Preferably, said cells expressing or abnormally expressing a tumor-associated nucleic acid and/or a tumor-associated antigen identified according to the invention are non-placenta cells.

The activity of a tumor-associated antigen identified according to the invention can be any activity of a protein or a peptide. In one embodiment this activity is an enzymatic activity.

According to the invention the phrase “inhibit expression or activity” includes a complete or essentially complete inhibition of expression or activity and a reduction in expression or activity.

The agent which, when administered, selectively increases the amount of complexes between an MHC molecule and a tumor-associated antigen identified according to the invention or a part thereof comprises one or more components selected from the group consisting of (i) the tumor-associated antigen or a part thereof, (ii) a nucleic acid which codes for said tumor-associated antigen or a part thereof, (iii) a host cell which expresses said tumor-associated antigen or a part thereof, and (iv) isolated complexes between peptide epitopes from said tumor-associated antigen and an MHC molecule.

The invention furthermore relates to a pharmaceutical composition which comprises one or more components selected from the group consisting of (i) a tumor-associated antigen identified according to the invention or a part thereof, (ii) a nucleic acid which codes for a tumor-associated antigen identified according to the invention or a part thereof, (iii) an antibody which binds to a tumor-associated antigen identified according to the invention or to a part thereof, (iv) an antisense nucleic acid which hybridizes specifically with a tumor-associated nucleic acid identified according to the invention/a nucleic acid coding for a tumor-associated antigen identified according to the invention, (v) an siRNA directed against a tumor-associated nucleic acid identified according to the invention/a nucleic acid coding for a tumor-associated antigen identified according to the invention, (vi) a host cell which expresses a tumor-associated antigen identified according to the invention or a part thereof, and (vii) isolated complexes between a tumor-associated antigen identified according to the invention or a part thereof and an MHC molecule.

In one embodiment, a nucleic acid coding for a tumor-associated antigen identified according to the invention or a part thereof is present in the pharmaceutical composition in an expression vector and functionally linked to a promoter. In a further embodiment, a nucleic acid coding for a tumor-associated antigen identified according to the invention or a part thereof is present in the pharmaceutical composition in a virus as further described below.

A host cell present in a pharmaceutical composition of the invention may secrete the tumor-associated antigen or the part thereof, may express it on the surface and preferably may additionally express an MHC molecule which binds to said tumor-associated antigen or said part thereof. In one embodiment, the host cell expresses the MHC molecule endogenously. In a further embodiment, the host cell expresses the MHC molecule and/or the tumor-associated antigen or the part thereof in a recombinant manner. The host cell is preferably nonproliferative. In a preferred embodiment, the host cell is an antigen-presenting cell, in particular a dendritic cell, a monocyte or a macrophage.

In a further embodiment, an antibody present in a pharmaceutical composition of the invention is a monoclonal antibody. In further embodiments, the antibody is a chimeric, human or humanized antibody, a fragment of an antibody or a synthetic antibody. The antibody may be coupled to a therapeutically or diagnostically useful agent also termed therapeutic or diagnostic agent or substance herein.

An antisense nucleic acid present in a pharmaceutical composition of the invention may comprise a sequence of 6-50, in particular 10-30, 15-30 and 20-30, contiguous nucleotides of the nucleic acid coding for the tumor-associated antigen identified according to the invention.

In further embodiments, a tumor-associated antigen or a part thereof, provided by a pharmaceutical composition of the invention either directly or via expression of a nucleic acid, binds to MHC molecules on the surface of cells, said binding preferably causing a cytolytic response and/or inducing cytokine release.

A pharmaceutical composition of the invention may comprise a pharmaceutically compatible carrier and/or an adjuvant.

A pharmaceutical composition of the invention is preferably used for the treatment or prevention of a disease characterized by selective expression or abnormal expression of a tumor-associated nucleic acid and/or tumor-associated antigen. In a preferred embodiment, the disease is a neoplastic disease, preferably cancer.

In a preferred embodiment, the pharmaceutical composition of the invention is in the form of a vaccine which may be used therapeutically or prophylactically. Such vaccine preferably comprises a tumor-associated antigen identified according to the invention or a part thereof, and/or a nucleic acid which codes for a tumor-associated antigen identified according to the invention or a part thereof. In particular embodiments, the nucleic acid is present in a virus or host cell.

The invention furthermore relates to methods of treating, preventing, diagnosing or monitoring, i.e. determining the regression, progression, course and/or onset of, a disease characterized by expression or abnormal expression of one of more tumor-associated nucleic acids identified according to the invention, preferably also resulting in expression or abnormal expression of one of more tumor-associated antigens identified according to the invention, preferably a neoplastic disease, in particular cancer. In one embodiment, the treatment or prevention comprises administering a pharmaceutical composition of the invention.

The methods of diagnosing and/or methods of monitoring according to the invention generally concern the detection of and/or determination of the quantity of one or more parameters selected from the group consisting of (i) a tumor-associated nucleic acid identified according to the invention, or a part thereof, (ii) a tumor-associated antigen identified according to the invention, or a part thereof, (iii) an antibody against a tumor-associated antigen identified according to the invention or a part thereof, and (iv) T lymphocytes, preferably cytotoxic or T helper lymphocytes, which are specific for a tumor-associated antigen identified according to the invention or a part thereof and/or a complex between the tumor-associated antigen or a part thereof and an MHC molecule, in a biological sample isolated from a patient, preferably from a patient having said disease, being suspected of having or falling ill with said disease or having a potential for said disease. Means for accomplishing said detection and/or determination of the quantity are described herein and will be apparent to the skilled person.

Preferably, the presence of said nucleic acid or said part thereof, said tumor-associated antigen or said part thereof, said antibody and/or said T lymphocytes and/or a quantity of said nucleic acid or said part thereof, said tumor-associated antigen or said part thereof, said antibody and/or said T lymphocytes which is increased compared to a patient and/or a tissue without said disease is indicative for the presence of said disease or a potential for a development of said disease.

The methods of diagnosing and/or monitoring of the invention also include embodiments wherein by detection or determination of the quantity of said nucleic acid or said part thereof, said tumor-associated antigen or said part thereof, said antibody and/or said T lymphocytes it is possible to assess and/or prognose the metastatic behavior of said disease, wherein, preferably, the presence of said nucleic acid or said part thereof, said tumor-associated antigen or said part thereof, said antibody and/or said T lymphocytes and/or a quantity of said nucleic acid or said part thereof, said tumor-associated antigen or said part thereof, said antibody and/or said T lymphocytes which is increased compared to a patient without said disease or without a metastasis of said disease is indicative for a metastatic behavior of said disease or a potential for a metastatic behavior of said disease.

In particular embodiments, said detection or determination of the quantity comprises (i) contacting a biological sample with an agent which binds specifically to said tumor-associated nucleic acid or said part thereof, to said tumor-associated antigen or said part thereof, to said antibody or to said T lymphocytes, and (ii) detecting the formation of or determining the amount of a complex between the agent and the nucleic acid or the part thereof, the tumor-associated antigen or the part thereof, the antibody, or the T lymphocytes.

In one embodiment, the disease is characterized by expression or abnormal expression of two or more different tumor-associated nucleic acids preferably also resulting in expression or abnormal expression of two or more different tumor-associated antigens and a detection or determination of the quantity comprises a detection or determination of the quantity of two or more different tumor-associated nucleic acids or of parts thereof, of two or more different tumor-associated antigens or of parts thereof, of two or more antibodies binding to said two or more different tumor-associated antigens or to parts thereof and/or of two or more T lymphocytes specific for said two or more different tumor-associated antigens or parts thereof, or complexes thereof with MHC molecules. In a further embodiment, the biological sample isolated from the patient is compared to a comparable normal biological sample.

The methods of monitoring according to the invention preferably comprise a detection of and/or determination of the quantity of one or more of the parameters mentioned above in a first sample at a first point in time and in a further sample at a second point in time, wherein the course of the disease is determined by comparing the two samples.

Preferably, a level of said nucleic acid or said part thereof, said tumor-associated antigen or said part thereof, said antibody and/or said T lymphocytes winch is increased in a sample compared to a sample taken earlier from a patient indicates that the patient has developed or is about to develop cancer and/or a metastasis of cancer and/or a relapse of cancer. Preferably, a level of said nucleic acid or said part thereof, said tumor-associated antigen or said part thereof, said antibody and/or said T lymphocytes which is decreased in a sample compared to a sample taken earlier from a patient indicates regression of cancer and/or a metastasis of cancer in said patient and thus, preferably indicates a successful cancer therapy.

According to the invention, detection of a nucleic acid or of a part thereof or determining the quantity of a nucleic acid or of a part thereof may be carried out using a oligo- or polynucleotide probe which hybridizes specifically to said nucleic acid or said part thereof or may be carried out by selective amplification of said nucleic acid or said part thereof, e.g. by means of PCR amplification. In one embodiment, the oligo- or polynucleotide probe comprises a sequence of 6-50, in particular 10-30, 15-30 and 20-30, contiguous nucleotides of said nucleic acid.

In particular embodiments, the tumor-associated antigen or the part thereof which is to be detected or the quantity of which is to be determined in the methods of the present invention is present intracellularly, on the cell surface or in a complex with an MHC molecule.

According to the invention, detection of a tumor-associated antigen or of a part thereof or determining the quantity of a tumor-associated antigen or of a part thereof may be carried out using an antibody binding specifically to said tumor-associated antigen or said part thereof.

According to the invention, detection of an antibody or determining the quantity of an antibody may be carried out using a protein or peptide binding specifically to said antibody.

According to the invention, detection of or determining the quantity of T lymphocytes which are specific for a tumor-associated antigen or a part thereof and/or a complex thereof with an MHC molecule may be carried out using a cell presenting the complex between said tumor-associated antigen or said part thereof and an MHC molecule. T lymphocytes may additionally be detected by detecting their proliferation, their cytokine production, and their cytotoxic activity triggered by specific stimulation with a complex of an MHC molecule and a tumor-associated antigen or a part thereof. T lymphocytes may also be detected with aid of a recombinant MHC molecule or a complex of two or more MHC molecules loaded with immunogenic fragments of one or more tumor-associated antigens.

An agent which is used for detection or determining the quantity in the methods of the invention such as a oligo- or polynucleotide probe, an antibody, a protein or peptide or a cell is preferably labeled in a detectable manner, in particular by a detectable marker such as a radioactive marker or an enzymic marker.

In a particular aspect, the invention relates to a method of treating, preventing, diagnosing or monitoring a disease characterized by expression or abnormal expression of a tumor-associated antigen identified according to the invention, which method comprises administering an antibody which binds to said tumor-associated antigen or to a part thereof and which is coupled to a therapeutic or diagnostic agent. The antibody may be a monoclonal antibody. In further embodiments, the antibody is a chimeric or humanized antibody or a fragment of an antibody.

In certain embodiments, the methods of the invention of diagnosing or monitoring a disease are performed with a biological sample containing or suspected of containing tumor cells such as disseminating tumor cells or metastatic tumor cells. Such biological samples include, for example, tissue, blood, serum, bone marrow, sputum, bronchial aspirate, and/or bronchial lavage. Preferably, the methods of the invention of diagnosing or monitoring a disease are performed with a biological sample not containing placental cells and, in particular, being a non-placenta biological sample isolated from a subject. In one embodiment, the biological sample is from a tissue or organ wherein the cells when the tissue or organ is free of tumors do not substantially express a tumor-associated antigen identified according to the invention and/or a tumor-associated nucleic acid identified according to the invention.

In one particular aspect, the invention relates to a method of treating a patient having a disease characterized by expression or abnormal expression of a tumor-associated antigen identified according to the invention, which method comprises (i) providing a sample containing immunoreactive cells, either obtained from said patient or from another individual of the same species, in particular a healthy individual, or an individual of a different species, (ii) contacting said sample with a host cell expressing said tumor-associated antigen or a part thereof, under conditions which favor production of cytolytic T cells against said tumor-associated antigen or a part thereof, and (iii) introducing the cytolytic T cells into the patient in an amount suitable for lysing cells expressing the tumor-associated antigen or a part thereof. In one embodiment, the method includes cloning of the T cell receptor of cytolytic T cells obtained and transferring the nucleic acid coding for the T cell receptor to T cells, either obtained from said patient or from another individual of the same species, in particular a healthy individual, or an individual of a different species, which T cells thus receive the desired specificity and, as under (iii), may be introduced into the patient.

In one embodiment, the host cell endogenously expresses an MHC molecule. In a further embodiment, the host cell recombinantly expresses an MHC molecule and/or the tumor-associated antigen or the part thereof. Preferably, the host cell presents the tumor-associated antigen or the part thereof by MHC molecules on its surface. The host cell is preferably nonproliferative. In a preferred embodiment, the host cell is an antigen-presenting cell, in particular a dendritic cell, a monocyte or a macrophage.

The invention also relates to a method of treating a disease characterized by expression or abnormal expression of a tumor-associated antigen identified according to the invention, which method comprises (i) identifying cells from the patient which express abnormal amounts of the tumor-associated antigen, (ii) isolating a sample of said cells, (iii) culturing said cells, and (iv) introducing said cells into the patient in an amount suitable for triggering an immune response to the cells.

The present invention furthermore relates to a nucleic acid selected from the group consisting of (a) a nucleic acid which comprises a nucleic acid sequence selected from the group consisting of SEQ ID NOs: 1-540, 541, 545, 549, 553, 557, 560, 563, 566, 570, 574, 577, 580, 583, 587, 591, 595, 599, 602, 606, 610, 613, 617, 620, 624, 634, 638, 642, 646, 649, 653, 656, 660, 664, 668, 671, 675, 679, 682, and 686, a part or derivative thereof, (b) a nucleic acid which hybridizes with the nucleic acid of (a) under stringent conditions, (c) a nucleic acid which is degenerate with respect to the nucleic acid of (a) or (b), and (d) a nucleic acid which is complementary to the nucleic acid of (a), (b) or (c).

In a further aspect, the invention relates to a recombinant nucleic acid molecule, in particular DNA or RNA molecule, which comprises a nucleic acid of the invention.

The invention also relates to host cells which contain a nucleic acid or recombinant nucleic acid molecule of the invention.

The host cell may also comprise a nucleic acid coding for a MHC molecule. In one embodiment, the host cell endogenously expresses the MHC molecule. In a further embodiment, the host cell recombinantly expresses the MHC molecule and/or the nucleic acid or recombinant nucleic acid molecule of the invention or a part thereof. Preferably, the host cell is nonproliferative. In a preferred embodiment, the host cell is an antigen-presenting cell, in particular a dendritic cell, a monocyte or a macrophage.

In a further embodiment, the invention relates to oligonucleotides which hybridize with a nucleic acid identified according to the invention and which may be used as genetic probes or as “antisense” molecules. Nucleic acid molecules in the form of oligonucleotide primers or competent probes, which hybridize with a nucleic acid identified according to the invention or parts thereof, may be used for detecting said nucleic acid and/or finding nucleic acids which are homologous to said nucleic acid identified according to the invention, e.g. by PCR amplification, Southern and Northern hybridization. Hybridization may be carried out under low stringency, more preferably under medium stringency and most preferably under high stringency conditions.

In a further aspect, the invention relates to a protein or peptide which is encoded by a nucleic acid selected from the group consisting of (a) a nucleic acid which comprises a nucleic acid sequence selected from the group consisting of SEQ ID NOs: 1-540, 541, 545, 549, 553, 557, 560, 563, 566, 570, 574, 577, 580, 583, 587, 591, 595, 599, 602, 606, 610, 613, 617, 620, 624, 634, 638, 642, 646, 649, 653, 656, 660, 664, 668, 671, 675, 679, 682, and 686, a part or derivative thereof, (b) a nucleic acid which hybridizes with the nucleic acid of (a) under stringent conditions, (c) a nucleic acid which is degenerate with respect to the nucleic acid of (a) or (b), and (d) a nucleic acid which is complementary to the nucleic acid of (a), (b) or (c). In a preferred embodiment, the protein or peptide comprises an amino acid sequence selected from the group consisting of SEQ ID NOs: 542, 546, 550, 554, 567, 571, 584, 588, 592, 596, 603, 607, 614, 621, 625, 635, 639, 643, 650, 657, 661, 665, 672, 676, and 683 of the sequence listing, a part or derivative thereof.

In a further aspect, the invention relates to an immunogenic fragment of a tumor-associated antigen identified according to the invention. Said fragment preferably binds to a MHC molecule or an antibody, preferably to a human HLA receptor or a human antibody. According to the invention, a part or fragment preferably comprises a sequence of at least 5, at least 6, in particular at least 8, at least 10, at least 12, at least 15, at least 20, at least 30 or at least 50, amino acids.

In a further aspect, the invention relates to an agent which binds to a tumor-associated antigen identified according to the invention or to a part thereof. In a preferred embodiment, the agent is a protein or peptide, in particular an antibody, a T cell receptor or an MHC molecule. In further embodiments, the antibody is a monoclonal, chimeric, human or humanized antibody, an antibody produced by combinatory techniques, or a fragment of an antibody. In one preferred embodiment, the invention relates to an antibody which binds selectively to a complex of (i) a tumor-associated antigen identified according to the invention or a part thereof and (ii) an MHC molecule to which said tumor-associated antigen identified according to the invention or said part thereof binds, with said antibody not binding to (i) or (ii) alone.

According to the invention, the term “binding” preferably relates to a specific binding. “Specific binding” means that an agent such as an antibody binds stronger to a target such as an epitope for which it is specific compared to the binding to another target. An agent binds stronger to a first target compared to a second target if it binds to the first target with a dissociation constant (K_{D}) which is lower than the dissociation constant for the second target. Preferably the dissociation constant (K_{D}) for the target to which the agent binds specifically is more than 10-fold, preferably more than 20-fold, more preferably more than 50-fold, even more preferably more than 100-fold, 200-fold, 500-fold or 1000-fold lower than the dissociation constant (K_{D}) for the target to which the agent does not bind specifically.

Such specific antibodies may, for example, be obtained by immunization using the aforementioned peptides.

The invention furthermore relates to a conjugate between an agent of the invention which binds to a tumor-associated antigen identified according to the invention or to a part thereof or an antibody of the invention and a therapeutic or diagnostic agent. In one embodiment, the therapeutic or diagnostic agent is a toxin.

In a further aspect, the invention relates to a kit for detecting a disease characterized by expression or abnormal expression of one of more tumor-associated nucleic acids identified according to the invention, preferably also resulting in expression or abnormal expression of one of more tumor-associated antigens identified according to the invention, preferably a neoplastic disease, in particular cancer, which kit comprises agents for detection or determining the quantity (i) of the tumor-associated nucleic acid or of a part thereof, (ii) of the tumor-associated antigen or of a part thereof, (iii) of antibodies which bind to the tumor-associated antigen or to a part thereof, and/or (iv) of T cells which are specific for the tumor-associated antigen or a part thereof or a complex thereof with an MHC molecule. Such agents are described herein above.

### DETAILED DESCRIPTION OF THE INVENTION

A reference herein to a range of numerical values is to be understood so as to specify and mention each of the individual numerical values comprised by said range. For example, a reference to SEQ ID NOs: 1-540 is to be understood so as to refer to each and every of the following individual SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, and 540.

According to the invention, a “reference” such as a reference sample or reference organism may be used to correlate and compare the results obtained in the methods of the invention from a test sample or test organism, i.e. a patient. Typically the reference sample is a sample from a healthy tissue, in particular a corresponding tissue which is not affected by cancer, or the reference organism is a healthy organism, in particular an organism which does not suffer from cancer.

A “reference value” can be determined from a reference empirically by measuring a sufficiently large number of references. Preferably the reference value is determined by measuring at least 2, preferably at least 3, preferably at least 5, preferably at least 8, preferably at least 12, preferably at least 20, preferably at least 30, preferably at least 50, or preferably at least 100 references.

According to the invention, a nucleic acid is preferably deoxyribonucleic acid (DNA) or ribonucleic acid (RNA). Nucleic acids comprise according to the invention genomic DNA, cDNA, mRNA, recombinantly produced and chemically synthesized molecules. According to the invention, a nucleic acid may be present as a single-stranded or double-stranded and linear or covalently circularly closed molecule.

The terms “tumor-associated nucleic acid identified according to the invention” and “nucleic acid encoding a tumor-associated antigen identified according to the invention” have similar meanings. However, the different terms are used herein to account for the fact that in some embodiments only the expression of nucleic acid, in particular mRNA, is of relevance while the expression of protein is not a critical factor.

As used herein, the term “RNA” means a molecule comprising at least one ribonucleotide residue. By “ribonucleotide” is meant a nucleotide with a hydroxyl group at the 2′-position of a beta-D-ribo-furanose moiety. The term includes double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

If reference is made herein to the detection of or the determination of the quantity of a nucleic acid, the nucleic acid which is actually to be detected or the quantity of which is actually to be determined is preferably mRNA. However, it should be understood that this may also include embodiments wherein mRNA is detected or the quantity of mRNA is determined indirectly. For example, mRNA may be transformed into cDNA and the cDNA detected or its quantity determined. mRNA is given herein as the cDNA equivalent. One skilled in the art would understand that the cDNA sequence is equivalent to the mRNA sequence, and can be used for the same purpose herein, e.g., the generation of probes hybridizing to the nucleic acid to be detected. Thus, if reference is made herein to the sequences shown in the sequence listing this is also to include the RNA equivalents of said sequences.

The nucleic acids described according to the invention have preferably been isolated. The term “isolated nucleic acid” means according to the invention that the nucleic acid was (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid which is available for manipulation by recombinant DNA techniques.

A degenerate nucleic acid according to the invention is a nucleic acid that differs from a reference nucleic acid in codon sequence due to the degeneracy of the genetic code.

The term “derivative” with respect to, for example, nucleic acid and amino acid sequences, according to the invention includes any variants, in particular mutants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. A species homolog is a nucleic acid or amino acid sequence with a different species of origin from that of a given nucleic acid or amino acid sequence.

A “derivative” of a nucleic acid according to the invention also includes nucleic acids comprising single or multiple such as at least 2, at least 4, or at least 6 and preferably up to 3, up to 4, up to 5, up to 6, up to 10, up to 15, or up to 20 nucleotide substitutions, deletions and/or additions. Furthermore, the term “derivative” also comprises chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

Preferably the degree of identity between a specific nucleic acid sequence described herein and a nucleic acid sequence which is a derivative of said specific nucleic acid sequence, which hybridizes with said specific nucleic acid sequence and/or which is degenerate with respect to said specific nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence, such as the nucleic acid sequences given in the sequence listing.

A nucleic acid is “complementary” to another nucleic acid if the two sequences are capable of hybridizing and forming a stable duplex with one another, with hybridization preferably being carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, N.Y., 1989 or Current Protocols in Molecular Biology, F. M. Ausubel et al., Editors, John Wiley & Sons, Inc., New York and refer, for example, to hybridization at 65° C. in hybridization buffer (3.5×SSC, 0.02% Ficoll, 0.02% polyvinylpyrrolidone, 0.02% bovine serum albumin, 2.5 mM NaH₂PO₄ (pH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68° C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the invention is at least 70%, preferably at least 75%, preferably at least 80%, more preferably at least 85%, even more preferably at least 90% or most preferably at least 95%, 96%, 97%, 98% or 99%. Most preferably, the degree of complementarity according to the invention is 100%.

“Sequence similarity” indicates the percentage of amino acids that either are identical or that represent conservative amino acid substitutions. “Sequence identity” between two polypeptide or nucleic acid sequences indicates the percentage of amino acids or nucleotides that are identical between the sequences.

The term “percentage identity” is intended to denote a percentage of nucleotides or of amino acid residues which are identical between the two sequences to be compared, obtained after the best alignment, this percentage being purely statistical and the differences between the two sequences being distributed randomly and over their entire length. Sequence comparisons between two nucleotide or amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

In one embodiment, a nucleic acid sequence which is a derivative of a specific nucleic acid sequence, which is degenerate with respect to a specific nucleic acid sequence or which is a part of a specific nucleic acid sequence has a relevant function and/or activity of the specific nucleic acid sequence, i.e. it may encode a protein or peptide having the same activity or immunological properties as the protein or peptide encoded by the specific nucleic acid sequence and, in one embodiment, encodes the same protein or peptide.

Nucleic acids coding for tumor-associated antigens may, according to the invention, be present alone or in combination with other nucleic acids, in particular heterologous nucleic acids. Preferably, a nucleic acid coding for a tumor-associated antigen or a part thereof expresses said tumor-associated antigen or part thereof. In preferred embodiments, a nucleic acid is functionally linked to expression control sequences or regulatory sequences which may be homologous or heterologous with respect to said nucleic acid. A coding sequence and a regulatory sequence are “functionally” linked to one another, if they are covalently linked to one another in such a way that expression or transcription of said coding sequence is under the control or under the influence of said regulatory sequence. If the coding sequence is to be translated into a functional protein, then, with a regulatory sequence functionally linked to said coding sequence, induction of said regulatory sequence results in transcription of said coding sequence, without causing a frame shift in the coding sequence or said coding sequence not being capable of being translated into the desired protein or peptide.

The term “expression control sequence” or “regulatory sequence” comprises according to the invention promoters, enhancers and other control elements which regulate expression of a gene. In particular embodiments of the invention, the expression control sequences can be regulated. The exact structure of regulatory sequences may vary as a function of the species or cell type, but generally comprises 5′ untranscribed and 5′ untranslated sequences which are involved in initiation of transcription and translation, respectively, such as TATA box, capping sequence, CAAT sequence, and the like. More specifically, 5′ untranscribed regulatory sequences comprise a promoter region which includes a promoter sequence for transcriptional control of the functionally linked gene. Regulatory sequences may also comprise enhancer sequences or upstream activator sequences.

According to the invention, a nucleic acid may furthermore be present in combination with another nucleic acid which codes for a peptide controlling secretion of the protein or peptide encoded by said nucleic acid from a host cell. According to the invention, a nucleic acid may also be present in combination with another nucleic acid which codes for a peptide causing the encoded protein or peptide to be anchored on the cell membrane of the host cell or compartmentalized into particular organelles of said cell. Similarly, a combination with a nucleic acid is possible which represents a reporter gene or any “tag”.

In a preferred embodiment, a recombinant nucleic acid molecule is according to the invention a vector, where appropriate with a promoter, which controls expression of a nucleic acid, for example a nucleic acid coding for a tumor-associated antigen identified according to the invention. The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. An intermediary vehicle may be adapted, for example, to the use in electroporation, in bombardment with microprojectiles, in liposomal administration, in the transfer with the aid of agrobacteria or in insertion via DNA or RNA viruses. Vectors comprise plasmids, phagemids, bacteriophages or viral genomes.

The nucleic acids coding for a tumor-associated antigen identified according to the invention may be used for transfection of host cells. Nucleic acids here mean both recombinant DNA and RNA. Recombinant RNA may be prepared by in-vitro transcription of a DNA template. Furthermore, it may be modified by stabilizing sequences, capping and polyadenylation prior to application.

According to the invention, the term “host cell” relates to any cell which can be transformed or transfected with an exogenous nucleic acid. The term “host cells” comprises according to the invention prokaryotic (e.g. E. coli) or eukaryotic cells (e.g. dendritic cells, B cells, CHO cells, COS cells, K562 cells, yeast cells and insect cells). Particular preference is given to mammalian cells such as cells from humans, mice, hamsters, pigs, goats, primates. The cells may be derived from a multiplicity of tissue types and comprise primary cells and cell lines. Specific examples comprise keratinocytes, peripheral blood leukocytes, stem cells of the bone marrow and embryonic stem cells. In further embodiments, the host cell is an antigen-presenting cell, in particular a dendritic cell, monocyte or a macrophage. A nucleic acid may be present in the host cell in the form of a single copy or of two or more copies and, in one embodiment, is expressed in the host cell.

According to the invention, the term “expression” is used in its most general meaning and comprises the production of RNA or of RNA and protein. It also comprises partial expression of nucleic acids. Furthermore, expression may be carried out transiently or stably. Preferred expression systems in mammalian cells comprise pcDNA3.1 and pRc/CMV (Invitrogen, Carlsbad, Calif.), which contain a selectable marker such as a gene imparting resistance to G418 (and thus enabling stably transfected cell lines to be selected) and the enhancer-promoter sequences of cytomegalovirus (CMV).

In those cases of the invention in which a MHC molecule presents a tumor-associated antigen or a part thereof, an expression vector may also comprise a nucleic acid sequence coding for said MHC molecule. The nucleic acid sequence coding for the MHC molecule may be present on the same expression vector as the nucleic acid coding for the tumor-associated antigen or the part thereof, or both nucleic acids may be present on different expression vectors. In the latter case, the two expression vectors may be cotransfected into a cell. If a host cell expresses neither the tumor-associated antigen or the part thereof nor the MHC molecule, both nucleic acids coding therefor may be transfected into the cell either on the same expression vector or on different expression vectors. If the cell already expresses the MHC molecule, only the nucleic acid sequence coding for the tumor-associated antigen or the part thereof can be transfected into the cell.

The invention also comprises kits for detection and/or determination of the quantity of nucleic acids. Such kits comprise, for example, a pair of amplification primers which hybridize to the nucleic acid which is to be detected or the amount of which is to be determined. The primers preferably comprise a sequence of 6-50, in particular 10-30, 15-30 and 20-30 contiguous nucleotides of the nucleic acid and are nonoverlapping, in order to avoid the formation of primer dimers. One of the primers will hybridize to one strand of the nucleic acid, and the other primer will hybridize to the complementary strand in an arrangement which allows amplification of the nucleic acid.

“Antisense molecules” or “antisense nucleic acids” may be used for regulating, in particular reducing, expression of a nucleic acid. The term “antisense molecule” or “antisense nucleic acid” refers according to the invention to an oligonucleotide which is an oligoribonucleotide, oligodeoxyribonucleotide, modified oligoribonucleotide or modified oligodeoxyribonucleotide and which hybridizes under physiological conditions to DNA comprising a particular gene or to mRNA of said gene, thereby inhibiting transcription of said gene and/or translation of said mRNA. According to the invention, an “antisense molecule” also comprises a construct which contains a nucleic acid or a part thereof in reverse orientation with respect to its natural promoter. An antisense transcript of a nucleic acid or of a part thereof may form a duplex with naturally occurring mRNA and thus prevent accumulation of or translation of the mRNA. Another possibility is the use of ribozymes for inactivating a nucleic acid.

Antisense oligonucleotides preferred according to the invention have a sequence of 6-50, in particular 10-30, 15-30 and 20-30, contiguous nucleotides of the target nucleic acid and preferably are fully complementary to the target nucleic acid or to a part thereof.

In preferred embodiments, the antisense oligonucleotide hybridizes with an N-terminal or 5′ upstream site such as a translation initiation site, transcription initiation site or promoter site. In further embodiments, the antisense oligonucleotide hybridizes with a 3′ untranslated region or mRNA splicing site.

In one embodiment, an oligonucleotide of the invention consists of ribonucleotides, deoxyribonucleotides or a combination thereof, with the 5′ end of one nucleotide and the 3′ end of another nucleotide being linked to one another by a phosphodiester bond. These oligonucleotides may be synthesized in the conventional manner or produced recombinantly.

In preferred embodiments, an oligonucleotide of the invention is a “modified” oligonucleotide. Here, the oligonucleotide may be modified in very different ways, without impairing its ability to bind its target, in order to increase, for example, its stability or therapeutic efficacy. According to the invention, the term “modified oligonucleotide” means an oligonucleotide in which (i) at least two of its nucleotides are linked to one another by a synthetic internucleoside bond (i.e. an internucleoside bond which is not a phosphodiester bond) and/or (ii) a chemical group which is usually not found in nucleic acids is covalently linked to the oligonucleotide. Preferred synthetic internucleoside bonds are phosphorothioates, alkyl phosphonates, phosphorodithioates, phosphate esters, alkyl phosphonothioates, phosphoramidates, carbamates, carbonates, phosphate triesters, acetamidates, carboxymethyl esters and peptides.

The term “modified oligonucleotide” also comprises oligonucleotides having a covalently modified base and/or sugar. “Modified oligonucleotides” comprise, for example, oligonucleotides with sugar residues which are covalently bound to low molecular weight organic groups other than a hydroxyl group at the 3′ position and a phosphate group at the 5′ position. Modified oligonucleotides may comprise, for example, a 2′-O-alkylated ribose residue or another sugar instead of ribose, such as arabinose.

It is to be understood that all embodiments described above with respect to oligonucleotides may also apply to polynucleotides.

By “small interfering RNA” or “siRNA” as used herein is meant an isolated RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is used to identify a target gene or mRNA to be degraded. A range of 19-25 nucleotides is the most preferred size for siRNAs.

siRNA according to the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more nucleotides in the siRNA with deoxyribonucleotides. Furthermore, siRNA may be modified to increase the stability thereof as described above for modified oligonucleotides, in particular by introducing one or more phosphorothioate linkages.

One or both strands of the siRNA can also comprise a 3′-overhang. As used herein, a “3′-overhang” refers to at least one unpaired nucleotide extending from the 3′-end of an RNA strand. Thus in one embodiment, the siRNA comprises at least one 3′-overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length. In the embodiment in which both strands of the siRNA molecule comprise a 3′-overhang, the length of the overhangs can be the same or different for each strand. In a most preferred embodiment, the 3′-overhang is present on both strands of the siRNA, and is 2 nucleotides in length. For example, each strand of the siRNA of the invention can comprise 3′-overhangs of dideoxythymidylic acid (“TT”) or diuridylic acid (“uu”).

In order to enhance the stability of the siRNA, the 3′-overhangs can be also stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′-overhangs with 2′-deoxythymidine, is tolerated and does not affect the efficiency of RNAi degradation. In particular, the absence of a 2′-hydroxyl in the 2′-deoxythymidine significantly enhances the nuclease resistance of the 3′-overhang in tissue culture medium.

The sense and antisense strands of the siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. Without wishing to be bound by any theory, it is believed that the hairpin area of the latter type of siRNA molecule is cleaved intracellularly by the “Dicer” protein (or its equivalent) to form a siRNA of two individual base-paired RNA molecules.

As used herein, “target mRNA” refers to an RNA molecule that is a target for downregulation.

siRNA can be expressed from pol III expression vectors without a change in targeting site, as expression of RNAs from pol III promoters is only believed to be efficient when the first transcribed nucleotide is a purine.

siRNA according to the invention can be targeted to any stretch of approximately 19-25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. “The siRNA User Guide” is available on the world wide web at a website maintained by Dr. Thomas Tuschl, Laboratory of RNA Molecular Biology, Rockefeller University, New York, USA, and can be found by accessing the website of the Rockefeller University and searching with the keyword “siRNA”. Thus, the sense strand of the present siRNA comprises a nucleotide sequence substantially identical to any contiguous stretch of about 19 to about 25 nucleotides in the target mRNA.

Generally, a target sequence on the target mRNA can be selected from a given cDNA sequence corresponding to the target mRNA, preferably beginning 50 to 100 nt downstream (i.e., in the 3′-direction) from the start codon. The target sequence can, however, be located in the 5′- or 3′-untranslated regions, or in the region nearby the start codon.

siRNA can be obtained using a number of techniques known to those of skill in the art. For example, siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, siRNA is chemically synthesized using appropriately protected ribonucleoside phosphoramidites and a conventional DNA/RNA synthesizer. siRNA can be synthesized as two separate, complementary RNA molecules, or as a single RNA molecule with two complementary regions.

Alternatively, siRNA can also be expressed from recombinant circular or linear DNA plasmids using any suitable promoter. Such embodiments are included according to the present invention when reference is made herein to the administration of siRNA or the incorporation of siRNA into pharmaceutical compositions. Suitable promoters for expressing siRNA of the invention from a plasmid include, for example, the U6 or H1 RNA pol III promoter sequences and the cytomegalovirus promoter.

Selection of other suitable promoters is within the skill in the art. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly. The use of recombinant plasmids to deliver siRNA to cells in vivo is discussed in more detail below. siRNA can be expressed from a recombinant plasmid either as two separate, complementary RNA molecules, or as a single RNA molecule with two complementary regions.

Selection of plasmids suitable for expressing siRNA, methods for inserting nucleic acid sequences for expressing the siRNA into the plasmid, and methods of delivering the recombinant plasmid to the cells of interest are within the skill in the art.

siRNA can also be expressed from recombinant viral vectors intracellularly in vivo. The recombinant viral vectors comprise sequences encoding the siRNA and any suitable promoter for expressing the siRNA sequences. The recombinant viral vectors can also comprise inducible or regulatable promoters for expression of the siRNA in a particular tissue or in a particular intracellular environment. siRNA can be expressed from a recombinant viral vector either as two separate, complementary RNA molecules, or as a single RNA molecule with two complementary regions.

The term “peptide” comprises oligo- and polypeptides and refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently by peptide bonds. The term “protein” refers to large peptides, preferably to peptides with more than 100 amino acid residues, but in general the terms “peptides” and “proteins” are synonyms and are used interchangeably herein.

Preferably, the proteins and peptides described according to the invention have been isolated. The terms “isolated protein” or “isolated peptide” mean that the protein or peptide has been separated from its natural environment. An isolated protein or peptide may be in an essentially purified state. The term “essentially purified” means that the protein or peptide is essentially free of other substances with which it is associated in nature or in vivo.

Such proteins and peptides may be used, for example, in producing antibodies and in an immunological or diagnostic assay or as therapeutics. Proteins and peptides described according to the invention may be isolated from biological samples such as tissue or cell homogenates and may also be expressed recombinantly in a multiplicity of pro- or eukaryotic expression systems.

For the purposes of the present invention, “derivatives” of a protein or peptide or of an amino acid sequence comprise amino acid insertion variants, amino acid deletion variants and/or amino acid substitution variants.

Amino acid insertion variants comprise amino- and/or carboxy-terminal fusions and also insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid deletion variants are characterized by the removal of one or more amino acids from the sequence.

Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties.

“Conservative substitutions” may be made, for instance, on the basis of similarity in polarity, charge, solubility, hydrophobicity, hydrophilicity, and/or the amphipathic nature of the residues involved. For example: (a) nonpolar (hydrophobic) amino acids include alanine, leucine, isoleucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

Preferably the degree of similarity, preferably identity between a specific amino acid sequence described herein and an amino acid sequence which is a derivative of said specific amino acid sequence will be at least 70%, preferably at least 80%, preferably at least 85%, even more preferably at least 90% or most preferably at least 95%, 96%, 97%, 98% or 99%. The degree of similarity or identity is given preferably for a region of at least about 20, at least about 40, at least about 60, at least about 80, at least about 100, at least about 120, at least about 140, at least about 160, at least about 200 or 250 amino acids. In preferred embodiments, the degree of similarity or identity is given for the entire length of the reference amino acid sequence.

The amino acid variants described above may be readily prepared with the aid of known peptide synthesis techniques such as, for example, by solid phase synthesis (Merrifield, 1964) and similar methods or by recombinant DNA manipulation. The manipulation of DNA sequences for preparing proteins and peptides having substitutions, insertions or deletions, is described in detail in Sambrook et al. (1989), for example.

According to the invention, “derivatives” of proteins and peptides include modified forms of proteins and peptides. Such modifications include any chemical modification and comprise single or multiple substitutions, deletions and/or additions of any molecules associated with the protein or peptide, such as carbohydrates, lipids and/or proteins or peptides. The term “derivative” also extends to all functional chemical equivalents of said proteins and peptides.

According to the invention, a part or fragment or derivative of a protein, peptide or nucleic acid, e.g. of a tumor-associated antigen or tumor-associated nucleic acid, preferably has a functional property of the protein, peptide or nucleic acid from which it has been derived. Such functional properties comprise the interaction with antibodies, the interaction with other peptides or proteins, the selective binding of nucleic acids and an enzymatic activity. In one embodiment, a part or fragment or derivative of a protein, peptide or nucleic acid is immunologically equivalent to the protein, peptide or nucleic acid from which it has been derived. In one embodiment, the functional property is an immunological property. A particular property is the ability to form a complex with MHC molecules and, where appropriate, generate an immune response, preferably by stimulating cytotoxic or T helper cells.

A part or fragment of a tumor-associated antigen or of an amino acid sequence described herein preferably comprises a sequence of at least 6, in particular at least 8, at least 10, at least 12, at least 15, at least 20, at least 30 or at least 50, consecutive amino acids of the tumor-associated antigen or of the amino acid sequence. A part or fragment of a tumor-associated antigen or of an amino acid sequence described herein preferably comprises a sequence of up to 8, in particular up to 10, up to 12, up to 15, up to 20, up to 30 or up to 55, consecutive amino acids of the tumor-associated antigen or of the amino acid sequence. A part or fragment of a tumor-associated antigen or of an amino acid sequence described herein is preferably a part of the tumor-associated antigen or of the amino acid sequence which corresponds to the non-transmembrane portion, in particular the extracellular portion of the antigen or amino acid sequence, or is comprised thereof. A part or fragment of a tumor-associated antigen or of an amino acid sequence described herein is preferably a part of the tumor-associated antigen or of the amino acid sequence which may be presented with MHC molecules and when so presented is capable of stimulating a cellular response.

A “cellular response” is meant to include a cellular response directed to cells characterized by presentation of a tumor-associated antigen with class I or class II MHC. The cellular response relates to cells called T cells or T lymphocytes which act as either ‘helpers’ or ‘killers’. The helper T cells (also termed CD4+ T cells) play a central role by regulating the immune response and the killer cells (also termed cytotoxic T cells, cytolytic T cells, CD8+ T cells or CTLs) kill tumor cells, preventing the production of more tumor cells. Although both arms of the immune response are thought to be necessary, the CTL response may be more important for controlling cancer.

By “cell characterized by presentation of a tumor-associated antigen with class I MHC” or “cell presenting a tumor-associated antigen with class I MHC” or similar expressions is meant a cell such as a tumor cell or an antigen presenting cell presenting the tumor-associated antigen it expresses or a fragment derived from said tumor-associated antigen, e.g. by processing of the tumor-associated antigen, in the context of MHC Class I molecules. Similarly, the term “tumor characterized by presentation of a tumor-associated antigen with class I MHC” denotes a tumor comprising cells characterized by presentation of a tumor-associated antigen with class I MHC.

Preferred parts or fragments of a tumor-associated antigen or of an amino acid sequence described herein are in particular suitable for the stimulation of cytotoxic T-lymphocytes in vivo but also for the production of expanded and stimulated T-lymphocytes for the therapeutic adoptive transfer ex vivo.

In one aspect, a part or fragment of a tumor-associated antigen or of an amino acid sequence described herein comprises an amino acid sequence derived from the sequence of the tumor-associated antigen or of the amino acid sequence. Preferably, a part or fragment of a tumor-associated antigen or of an amino acid sequence described herein is capable of stimulating a cellular response against cells characterized by presentation of a tumor-associated antigen identified according to the invention with class I MHC and/or of elicting antibodies that specifically bind to a tumor-associated antigen identified according to the invention when used itself or attached to an immunogenic carrier. Preferably, a part or fragment of a tumor-associated antigen or of an amino acid sequence described herein may be presented, directly or following processing, with class I MHC molecules. Preferably, a part or fragment of a tumor-associated antigen or of an amino acid sequence described herein is a MHC class I and/or class II presented peptide or can be processed to produce a MHC class I and/or class II presented peptide. Preferably, a part or fragment of a tumor-associated antigen or of an amino acid sequence described herein comprises an amino acid sequence substantially corresponding to the amino acid sequence of a fragment of a tumor-associated antigen or of an amino acid sequence described herein.

A part or fragment of a tumor-associated antigen or of an amino acid sequence described herein may be present according to the invention as a peptide or peptide antigen as such or in combination with additional amino acid sequences, i.e. it may be comprised in a peptide or peptide antigen. Such peptides or peptide antigens are useful in the compositions and methods of the present invention and generally are included herein by the term “tumor-associated antigen”. In general, such peptides or peptide antigens relate to oligopeptides or polypeptides comprising an amino acid sequence substantially corresponding to the amino acid sequence of a part or fragment of a tumor-associated antigen or of an amino acid sequence described herein. Preferably, such peptides or peptide antigens are capable of stimulating a cellular response against a tumor characterized by presentation of a tumor-associated antigen identified herein with class I MHC and/or of eliciting antibodies that specifically bind to a tumor-associated antigen identified according to the present invention.

If a peptide or peptide antigen is to be presented directly, i.e. without processing, in particular without cleavage, it has a length which is suitable for binding to an MHC molecule, in particular a class I MHC molecule, and preferably is 7-20 amino acids in length, more preferably 7-12 amino acids in length, more preferably 8-11 amino acids in length, in particular 9 or 10 amino acids in length. Preferably the sequence of a peptide or peptide antigen which is to be presented directly is derived from the amino acid sequence of a tumor-associated antigen or of an amino acid sequence described herein, i.e. its sequence substantially corresponds and is preferably completely identical to a fragment of a tumor-associated antigen or of an amino acid sequence described herein. If a peptide or peptide antigen is to be presented following processing, in particular following cleavage, the peptide produced by processing has a length which is suitable for binding to an MHC molecule, in particular a class I MHC molecule, and preferably is 7-20 amino acids in length, more preferably 7-12 amino acids in length, more preferably 8-11 amino acids in length, in particular 9 or 10 amino acids in length. Preferably the sequence of the peptide which is to be presented following processing is derived from the amino acid sequence of a tumor-associated antigen or of an amino acid sequence described herein, i.e. its sequence substantially corresponds and is preferably completely identical to a fragment of a tumor-associated antigen or of an amino acid sequence described herein. Thus, a peptide or peptide antigen according to the invention in one embodiment comprises a sequence of 7-20 amino acids in length, more preferably 7-12 amino acids in length, more preferably 8-11 amino acids in length, in particular 9 or 10 amino acids in length which substantially corresponds and is preferably completely identical to a fragment of a tumor-associated antigen or of an amino acid sequence described herein and following processing of the peptide or peptide antigen makes up the presented peptide. However, the peptide or peptide antigen may also comprise a sequence which substantially corresponds and preferably is completely identical to a fragment of a tumor-associated antigen or of an amino acid sequence described herein which is even longer than the above stated sequence. In one embodiment, a peptide or peptide antigen may comprise the entire sequence of a tumor-associated antigen or of an amino acid sequence described herein.

Peptides having amino acid sequences differing at residues that do not affect TCR recognition but improve the stability of binding to MHC may improve the immunogenicity of the peptide or peptide antigen, and may be referred to herein as “optimized peptides”. Using existing knowledge about which of these residues may be more likely to affect binding either to the MHC or to the TCR, a rational approach to the design of substantially corresponding peptides may be employed. Resulting peptides that are functional are contemplated herein.

In one embodiment, a part or a fragment of a nucleic acid coding for a tumor-associated antigen relates according to the invention to the part of the nucleic acid, which codes at least for the tumor-associated antigen and/or for a part or a fragment of said tumor-associated antigen, as defined above. A part or fragment of a nucleic acid coding for a tumor-associated antigen is preferably that part of the nucleic acid corresponding to the open reading frame.

According to the invention, particular embodiments ought to involve providing “dominant negative” proteins or peptides derived from tumor-associated antigens. A dominant negative protein or peptide is an inactive protein or peptide variant which, by way of interacting with the cellular machinery, displaces an active protein or peptide from its interaction with the cellular machinery or which competes with the active protein or peptide, thereby reducing the effect of said active protein.

Antisera which contain specific antibodies specifically binding to the target protein can be prepared by various standard processes; see, for example, “Monoclonal Antibodies: A Practical Approach” by Philip Shepherd, Christopher Dean ISBN 0-19-963722-9; “Antibodies: A Laboratory Manual” by Ed Harlow, David Lane, ISBN: 0879693142 and “Using Antibodies: A Laboratory Manual: Portable Protocol NO” by Edward Harlow, David Lane, Ed Harlow ISBN 0879695447. Thereby it is also possible to generate affine and specific antibodies which recognize complex membrane proteins in their native form (Azorsa et al., J. Immunol. Methods 229: 35-48, 1999; Anderson et al., J. Immunol. 143: 1899-1904, 1989; Gardsvoll, J. Immunol. Methods 234: 107-116, 2000). This is in particular relevant for the preparation of antibodies which are to be used therapeutically, but also for many diagnostic applications. In this respect, it is possible to immunize with the whole protein, with extracellular partial sequences as well as with cells which express the target molecule in physiologically folded form.

Monoclonal antibodies are traditionally prepared using the hybridoma technology. (for technical details see: “Monoclonal Antibodies: A Practical Approach” by Philip Shepherd, Christopher Dean ISBN 0-19-963722-9; “Antibodies: A Laboratory Manual” by Ed Harlow, David Lane ISBN: 0879693142; “Using Antibodies: A Laboratory Manual: Portable Protocol NO” by Edward Harlow, David Lane, Ed Harlow ISBN: 0879695447).

It is known that only a small part of an antibody molecule, the paratope, is involved in binding of the antibody to its epitope (cf. Clark, W. R. (1986), The Experimental Foundations of Modern Immunology, Wiley & Sons, Inc., New York; Roitt, I. (1991), Essential Immunology, 7th Edition, Blackwell Scientific Publications, Oxford). The pFc′ and Fc regions are, for example, effectors of the complement cascade but are not involved in antigen binding. An antibody from which the pFc′ region has been enzymatically removed or which has been produced without the pFc′ region, referred to as F(ab′)₂ fragment, carries both antigen binding sites of a complete antibody. Similarly, an antibody from which the Fc region has been enzymatically removed or which has been produced without said Fc region, referred to as Fab fragment, carries one antigen binding site of an intact antibody molecule. Furthermore, Fab fragments consist of a covalently bound light chain of an antibody and part of the heavy chain of said antibody, referred to as Fd. The Fd fragments are the main determinants of antibody specificity (a single Fd fragment can be associated with up to ten different light chains, without altering the specificity of the antibody) and Fd fragments, when isolated, retain the ability to bind to an epitope.

Located within the antigen-binding part of an antibody are complementary-determining regions (CDRs) which interact directly with the antigen epitope and framework regions (FRs) which maintain the tertiary structure of the paratope. Both the Fd fragment of the heavy chain and the light chain of IgG immunoglobulins contain four framework regions (FR1 to FR4) which are separated in each case by three complementary-determining regions (CDR1 to CDR3). The CDRs and, in particular, the CDR3 regions and, still more particularly, the CDR3 region of the heavy chain are responsible to a large extent for antibody specificity.

Non-CDR regions of a mammalian antibody are known to be able to be replaced by similar regions of antibodies with the same or a different specificity, with the specificity for the epitope of the original antibody being retained. This made possible the development of “humanized” antibodies in which nonhuman CDRs are covalently linked to human FR and/or Fc/pFc′ regions to produce a functional antibody.

As another example, WO 92/04381 describes the production and use of humanized murine RSV antibodies in which at least part of the murine FR regions have been replaced with FR regions of a human origin. Antibodies of this kind, including fragments of intact antibodies with antigen-binding capability, are often referred to as “chimeric” antibodies.

According to the invention, the term “antibody” also includes F(ab′)₂, Fab, Fv, and Fd fragments of antibodies, chimeric antibodies, in which the Fc and/or FR and/or CDR1 and/or CDR2 and/or light chain-CDR3 regions have been replaced with homologous human or nonhuman sequences, chimeric F(ab′)₂-fragment antibodies in which the FR and/or CDR1 and/or CDR2 and/or light chain-CDR3 regions have been replaced with homologous human or nonhuman sequences, chimeric Fab-fragment antibodies in which the FR and/or CDR1 and/or CDR2 and/or light chain-CDR3 regions have been replaced with homologous human or nonhuman sequences, and chimeric Fd-fragment antibodies in which the FR and/or CDR1 and/or CDR2 regions have been replaced with homologous human or nonhuman sequences. The term “antibody” also comprises “single-chain” antibodies.

The invention also comprises proteins and peptides which bind specifically to tumor-associated antigens. Binding substances of this kind may be provided, for example, by degenerate peptide libraries which may be prepared simply in solution in an immobilized form or as phage-display libraries. It is likewise possible to prepare combinatorial libraries of peptides with one or more amino acids. Libraries of peptoids and nonpeptidic synthetic residues may also be prepared.

Antibodies may also be coupled to specific diagnostic substances for displaying cells and tissues expressing tumor-associated antigens. They may also be coupled to therapeutically useful substances.

Diagnostic substances or agents include any label that functions to: (i) provide a detectable signal; (ii) interact with a second label to modify the detectable signal provided by the first or second label, e.g. FRET (Fluorescence Resonance Energy Transfer); (iii) affect mobility, e.g. electrophoretic mobility, by charge, hydrophobicity, shape, or other physical parameters, or (iv) provide a capture moiety, e.g., affinity, antibody/antigen, or ionic complexation. Suitable as label are structures, such as fluorescent labels, luminescent labels, chromophore labels, radioisotopic labels, isotopic labels, preferably stable isotopic labels, isobaric labels, enzyme labels, particle labels, in particular metal particle labels, magnetic particle labels, polymer particle labels, small organic molecules such as biotin, ligands of receptors or binding molecules such as cell adhesion proteins or lectins, label-sequences comprising nucleic acids and/or amino acid residues which can be detected by use of binding agents, etc. Diagnostic substances comprise, in a nonlimiting manner, barium sulfate, iocetamic acid, iopanoic acid, calcium ipodate, sodium diatrizoate, meglumine diatrizoate, metrizamide, sodium tyropanoate and radio diagnostic, including positron emitters such as fluorine-18 and carbon-11, gamma emitters such as iodine-123, technetium-99m, iodine-131 and indium-111, nuclides for nuclear magnetic resonance, such as fluorine and gadolinium.

According to the invention, the terms “therapeutically useful substance”, “therapeutic substance” or “therapeutic agent” means any molecule which may exert a therapeutic effect. According to the invention, a therapeutically useful substance is preferably selectively guided to a cell which expresses one or more tumor-associated antigens and includes anticancer agents, radioactive compounds such as radioactive iodine-labeled compounds, toxins, cytostatic or cytolytic drugs, etc. Anticancer agents comprise, for example, aminoglutethimide, azathioprine, bleomycin sulfate, busulfan, carmustine, chlorambucil, cisplatin, cyclophosphamide, cyclosporine, cytarabidine, dacarbazine, dactinomycin, daunorubin, doxorubicin, taxol, etoposide, fluorouracil, interferon-α, lomustine, mercaptopurine, methotrexate, mitotane, procarbazine HCl, thioguanine, vinblastine sulfate and vincristine sulfate. Other anticancer agents are described, for example, in Goodman and Gilman, “The Pharmacological Basis of Therapeutics”, 8th Edition, 1990, McGraw-Hill, Inc., in particular Chapter 52 (Antineoplastic Agents (Paul Calabresi and Bruce A. Chabner). Toxins may be proteins such as pokeweed antiviral protein, cholera toxin, pertussis toxin, ricin, gelonin, abrin, diphtheria exotoxin or Pseudomonas exotoxin. Toxin residues may also be high energy-emitting radionuclides such as cobalt-60.

The term “major histocompatibility complex” or “MHC” relates to a complex of genes present in all vertebrates. MHC proteins or molecules are involved in signaling between lymphocytes and antigen presenting cells in normal immune reactions by binding peptides and presenting them for recognition by T cell receptors (TCR). MHC molecules bind peptides within an intracellular processing compartment and present these peptides on the surface of antigen presenting cells for recognition by T cells. The human MHC region also termed HLA is located on chromosome 6 and includes the class I and class II region. In one preferred embodiment of all aspects of the invention an MHC molecule is an HLA molecule.

“Reduce” or “inhibit” as used herein means the ability to cause an overall decrease, preferably of 20% or greater, more preferably of 50% or greater, and most preferably of 75% or greater, in the level, e.g. in the level of protein or mRNA as compared to a reference sample (e.g., a sample not treated with siRNA). This reduction or inhibition of RNA or protein expression can occur through targeted mRNA cleavage or degradation. Assays for protein expression or nucleic acid expression are known in the art and include, for example, ELISA, western blot analysis for protein expression, and northern blotting or RNase protection assays for RNA.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

According to the invention the term “increased” or “increased amount” preferably refers to an increase by at least 10%, in particular at least 20%, at least 50% or at least 100%. The amount of a substance is also increased in a test sample such as a biological sample compared to a reference sample if it is detectable in the test sample but absent or not detectable in the reference sample.

According to the invention, the term “disease” refers to any pathological state in which tumor-associated nucleic acids and/or tumor-associated antigens are expressed or abnormally expressed. According to the invention, the term “tumor” or “tumor disease” refers to a swelling or lesion formed by an abnormal growth of cells (called neoplastic cells or tumor cells). By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant. Preferably, a tumor disease according to the invention is a cancer disease, i.e. a malignant disease and a tumor cell is a cancer cell. Preferably, a tumor disease is characterized by cells in which a tumor-associated nucleic acid and/or tumor-associated antigen identified according to the invention is expressed or abnormally expressed and a tumor cell or a circulating or metastatic tumor cell is characterized by expression or abnormal expression of a tumor-associated nucleic acid and/or tumor-associated antigen identified according to the invention. Preferably, a tumor disease, a tumor cell or a circulating or metastatic tumor cell is characterized by presentation of a tumor-associated antigen identified according to the invention with class I MHC.

“Abnormal expression” means according to the invention that expression is altered, preferably increased, compared to the state in a healthy individual. An increase in expression refers to an increase by at least 10%, in particular at least 20%, at least 50% or at least 100%. In one embodiment, expression is only found in a diseased tissue, while expression in a healthy tissue is repressed or essentially repressed. One example of such a disease is cancer, wherein the term “cancer” according to the invention comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the matastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

According to the invention, a biological sample may be a tissue sample, including bodily fluids, and/or a cellular sample and may be obtained in the conventional manner such as by tissue biopsy, including punch biopsy, and by taking blood, bronchial aspirate, sputum, urine, feces or other body fluids. In one embodiment, a biological sample is obtained from a tissue for which a connection between expression of the target nucleic acids or target antigens identified herein and a cancer disease is demonstrated herein. In one embodiment, a biological sample is obtained from a tissue for which overexpression of the target nucleic acids or target antigens identified herein in a cancer tissue compared to normal tissues is demonstrated herein. In this embodiment, the diagnostic methods disclosed herein aim at diagnosing cancer of said tissue. For example, in the case of lung cancer, the sample may be a sample comprising lung tissue. According to the invention, the term “biological sample” also includes processed biological samples such as fractions or isolates of biological samples, e.g. nucleic acid and peptide/protein isolates. Preferably, the term “biological sample” according to the invention does not include samples derived from placental tissue or samples derived from tissues which demonstrate expression of the target nucleic acids or target antigens identified herein even if not affected by cancer.

Some aspects of the present invention envision the immunotherapy of tumor diseases, in particular cancer diseases, utilizing the tumor-associated nucleic acids and tumor-associated antigens identified according to the invention by means of active or passive immunotherapeutioc approaches which can be summarized as follows:

### Immunotherapy

I. Active immunotherapy (“Cancer vaccines”)

Immunisation with:

i) antigen or peptide (native or modified)
ii) nucleic acid encoding the antigen or peptide
iii) recombinant cells encoding the antigen or peptide
iv) recombinant viruses encoding the antigen or peptide
v) antigen presenting cells pulsed with antigen or peptide (native or modified) or transfected with nucleic acids encoding the antigen or peptide
II. Passive immunotherapy (“Adoptive immunotherapy”)
vi) Transfer of antibodies or T cell receptors that recognise antigen
vii) Transfer of cells sensitized in vitro to antigen (bulk or cloned populations)
viii) Transfer of effector cells (or stem cells) transduced with nucleic acids encoding T cell receptors that recognise antigen and preferably are responsive to tumor-specific class I MHC presented peptides

Antigen presenting cells (APC) such as dendritic cells (DCs) can be loaded with either MHC class I-presented peptide antigens or tumor lysate, or transduced with nucleic acid such as by transduction using adenovirus encoding a tumor-associated antigen, in particular a peptide antigen.

In a preferred embodiment, an anti-tumor vaccine of the invention comprises an APC loaded with peptide antigen. In this respect, protocols may rely on in vitro culture/differentiation of DCs manipulated in such a way that they artificially present peptide antigen. Production of genetically engineered DCs may involve introduction of nucleic acids encoding tumor-associated antigens or peptide antigens into DCs. Transfection of DCs with mRNA is a promising antigen-loading technique of stimulating strong antitumor immunity.

If used for passive anti-tumor immunotherapy, antibodies may or may not be attached to therapeutic effector moieties, e.g., radiolabels, cytotoxins, therapeutic enzymes, agents that induce apoptosis, and the like in order to provide for targeted cytotoxicity, i.e., killing of tumor cells. In one embodiment of the present invention, such antibodies or fragments are administered in labeled or unlabeled form, alone or in conjunction with other therapeutics, e.g., chemotherapeutics such as cisplatin, methotrexate, adriamycin, and the like suitable for cancer therapy.

Preferably the antibodies described herein mediate killing of cells by inducing complement dependent cytotoxicity (CDC) mediated lysis, antibody dependent cellular cytotoxicity (ADCC) mediated lysis, apoptosis, homotypic adhesion, and/or phagocytosis, preferably by inducing CDC mediated lysis and/or ADCC mediated lysis. The antibodies described herein preferably interact with components of the immune system, preferably through ADCC or CDC. However, antibodies of the invention may also exert an effect simply by binding to tumor-associated antigens on the cell surface, thus, e.g. blocking proliferation of the cells.

ADCC describes the cell-killing ability of effector cells as described herein, in particular lymphocytes, which preferably requires the target cell being marked by an antibody.

ADCC preferably occurs when antibodies bind to antigens on tumor cells and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that also leads to antigen presentation and the induction of tumor-directed T-cell responses. Preferably, in vivo induction of ADCC will lead to tumor-directed T-cell responses and host-derived antibody responses.

CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the C_{H}2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell and may lead to apoptosis.

Passive immunotherapy with immune cells (optionally genetically modified) capable of recognizing tumor-associated antigens is effective in mediating the regression of cancer in selected patients. These techniques may be based on ex-vivo reactivation and expansion of cloned or polyclonal cultures of tumor reactive T cells. After culture, T cells may be reinfused into the patient along with IL-2. In vitro techniques have been developed in which human lymphocytes are sensitized in vitro to tumor peptide antigens presented on antigen presenting cells. By repetitive in vitro stimulation cells can be derived with a great capacity to recognize human tumor-associated antigens. The adoptive transfer of these cells may be more effective in mediating tumor regression in vivo than are conventionally grown cells.

According to the invention, the term “immunoreactive cell” means a cell which can mature into an immune cell (such as B cell, T helper cell, or cytolytic T cell) with suitable stimulation. Immunoreactive cells comprise CD34⁺ hematopoietic stem cells, immature and mature T cells and immature and mature B cells. If production of cytolytic or T helper cells recognizing a tumor-associated antigen is desired, the immunoreactive cell is contacted with a cell expressing a tumor-associated antigen under conditions which favor production, differentiation and/or selection of cytolytic T cells and of T helper cells. The differentiation of T cell precursors into a cytolytic T cell, when exposed to an antigen, is similar to clonal selection of the immune system.

The terms “T cell” and “T lymphocyte” are used interchangeably herein and include T helper cells and cytotoxic T cells which comprise cytolytic T cells.

Some therapeutic methods are based on a reaction of the immune system of a patient, which results in a lysis of antigen-presenting cells such as cancer cells which present one or more tumor-associated antigens. In this connection, for example autologous cytotoxic T lymphocytes specific for a complex of a tumor-associated antigen and an MHC molecule are administered to a patient having a cellular abnormality. The production of such cytotoxic T lymphocytes in vitro is known. An example of a method of differentiating T cells can be found in WO-A-9633265. Generally, a sample containing cells such as blood cells is taken from the patient and the cells are contacted with a cell which presents the complex and which can cause propagation of cytotoxic T lymphocytes (e.g. dendritic cells). The target cell may be a transfected cell such as a COS cell. These transfected cells present the desired complex on their surface and, when contacted with cytotoxic T lymphocytes, stimulate propagation of the latter. The clonally expanded autologous cytotoxic T lymphocytes are then administered to the patient.

In another method of selecting antigen-specific cytotoxic T lymphocytes, fluorogenic tetramers of MHC class I molecule/peptide complexes are used for obtaining specific clones of cytotoxic T lymphocytes (Altman et al., Science 274:94-96, 1996; Dunbar et al., Curr. Biol. 8:413-416, 1998).

The present invention also includes therapeutic methods referred to as adoptive transfer (Greenberg, J. Immunol. 136(5):1917, 1986; Riddel et al., Science 257:238, 1992; Lynch et al., Eur. J. Immunol. 21:1403-1410, 1991; Kast et al., Cell 59:603-614, 1989), wherein cells presenting the desired complex (e.g. dendritic cells) are combined with cytotoxic T lymphocytes of the patient to be treated, resulting in a propagation of specific cytotoxic T lymphocytes. The propagated cytotoxic T lymphocytes are then administered to a patient having a cellular anomaly characterized by particular abnormal cells presenting the specific complex. The cytotoxic T lymphocytes then lyse the abnormal cells, thereby achieving a desired therapeutic effect.

Furthermore, cells presenting the desired complex (e.g. dendritic cells) may be combined with cytotoxic T lymphocytes of healthy individuals or another species (e.g. mouse) which may result in propagation of specific cytotoxic T lymphocytes with high affinity. The high affinity T cell receptor of these propagated specific T lymphocytes may be cloned and optionally humanized to a different extent, and the T cell receptors thus obtained then transduced via gene transfer, for example using retroviral vectors, into T cells of patients. Adoptive transfer may then be carried out using these genetically altered T lymphocytes (Stanislawski et al., Nat. Immunol. 2:962-70, 2001; Kessels et al., Nat. Immunol. 2:957-61, 2001).

Adoptive transfer is not the only form of therapy which can be applied according to the invention. Cytotoxic T lymphocytes may also be generated in vivo in a manner known per se. One method uses nonproliferative cells expressing the complex. The cells used here will be those which usually express the complex, such as irradiated tumor cells or cells transfected with one or both genes necessary for presentation of the complex (i.e. the antigenic peptide and the presenting MHC molecule). Another preferred form is the introduction of the tumor-associated antigen in the form of recombinant RNA which may be introduced into cells by liposomal transfer or by electroporation, for example. The resulting cells present the complex of interest and are recognized by autologous cytotoxic T lymphocytes which then propagate.

A similar effect can be achieved by combining the tumor-associated antigen or a fragment thereof with an adjuvant in order to make incorporation into antigen-presenting cells in vivo possible. The tumor-associated antigen or a fragment thereof may be represented as protein, as DNA (e.g. within a vector) or as RNA. The tumor-associated antigen is processed to produce a peptide partner for the MHC molecule, while a fragment thereof may be presented without the need for further processing. The latter is the case in particular, if these can bind to MI-IC molecules. Preference is given to administration forms in which the complete antigen is processed in vivo by a dendritic cell, since this may also produce T helper cell responses which are needed for an effective immune response (Ossendorp et al., Immunol Lett. 74:75-9, 2000; Ossendorp et al., J. Exp. Med. 187:693-702, 1998). In general, it is possible to administer an effective amount of the tumor-associated antigen to a patient by intradermal injection, for example. However, injection may also be carried out intranodally into a lymph node (Maloy et al., Proc Natl Acad Sci USA 98:3299-303, 2001).

The pharmaceutical compositions and methods of treatment described according to the invention may also be used for immunization or vaccination to therapeutically treat or prevent a disease described herein. According to the invention, the terms “immunization” or “vaccination” preferably relate to an increase in or activation of an immune response to an antigen. It is possible to use animal models for testing an immunizing effect on cancer by using a tumor-associated antigen or a nucleic acid coding therefor. For example, human cancer cells may be introduced into a mouse to generate a tumor, and one or more nucleic acids coding for tumor-associated antigens may be administered. The effect on the cancer cells (for example reduction in tumor size) may be measured as a measure for the effectiveness of an immunization by the nucleic acid.

As part of the composition for an immunization or a vaccination, preferably one or more tumor-associated antigens or stimulating fragments thereof are administered together with one or more adjuvants for inducing an immune response or for increasing an immune response. An adjuvant is a substance which is incorporated into the antigen or administered together with the latter and which enhances the immune response. Adjuvants may enhance the immune response by providing an antigen reservoir (extracellularly or in macrophages), activating macrophages and/or stimulating particular lymphocytes. Adjuvants are known and comprise in a nonlimiting way monophosphoryl lipid A (MPL, SmithKline Beecham), saponins such as QS21 (SmithKline Beecham), DQS21 (SmithKline Beecham; WO 96/33739), QS7, QS17, QS18 and QS-L1 (So et al., Mol. Cells 7:178-186, 1997), incomplete Freund's adjuvant, complete Freund's adjuvant, vitamin E, montanide, alum, CpG oligonucleotides (cf. Kreig et al., Nature 374:546-9, 1995) and various water-in-oil emulsions prepared from biologically degradable oils such as squalene and/or tocopherol. Preferably, the peptides are administered in a mixture with DQS21/MPL. The ratio of DQS21 to MPL is typically about 1:10 to 10:1, preferably about 1:5 to 5:1 and in particular about 1:1. For administration to humans, a vaccine formulation typically contains DQS21 and MPL in a range from about 1 μg to about 100 μg.

Other substances which stimulate an immune response of the patient may also be administered. It is possible, for example, to use cytokines in a vaccination, owing to their regulatory properties on lymphocytes. Such cytokines comprise, for example, interleukin-12 (IL-12) which was shown to increase the protective actions of vaccines (cf. Science 268:1432-1434, 1995), GM-CSF and IL-18.

There are a number of compounds which enhance an immune response and which therefore may be used in a vaccination. Said compounds comprise costimulating molecules provided in the form of proteins or nucleic acids such as B7-1 and B7-2 (CD80 and CD86, respectively).

The invention also provides for administration of nucleic acids, proteins or peptides. Proteins and peptides may be administered in a manner known per se. In one embodiment, nucleic acids are administered by ex vivo methods, i.e. by removing cells from a patient, genetic modification of said cells in order to incorporate a tumor-associated antigen and reintroduction of the altered cells into the patient. This generally comprises introducing a functional copy of a gene into the cells of a patient in vitro and reintroducing the genetically altered cells into the patient. The functional copy of the gene is under the functional control of regulatory elements which allow the gene to be expressed in the genetically altered cells. Transfection and transduction methods are known to the skilled worker. The invention also provides for administering nucleic acids in vivo by using vectors such as viruses and target-controlled liposomes. If according to the invention reference is made to the administration or incorporation into pharmaceutical compositions of nucleic acids this includes embodiments wherein the nucleic acid is present in such vectors.

In a preferred embodiment, a virus or viral vector for administering a nucleic acid coding for a tumor-associated antigen is selected from the group consisting of adenoviruses, adeno-associated viruses, pox viruses, including vaccinia virus and attenuated pox viruses, Semliki Forest virus, retroviruses, Sindbis virus and Ty virus-like particles. Particular preference is given to adenoviruses and retroviruses. The retroviruses are typically replication-deficient (i.e. they are incapable of generating infectious particles).

Methods of introducing nucleic acids into cells in vitro or in vivo comprise transfection of nucleic acid calcium phosphate precipitates, transfection of nucleic acids associated with DEAE, transfection or infection with the above viruses carrying the nucleic acids of interest, liposome-mediated transfection, and the like. In particular embodiments, preference is given to directing the nucleic acid to particular cells. In such embodiments, a carrier used for administering a nucleic acid to a cell (e.g. a retrovirus or a liposome) may have a bound target control molecule. For example, a molecule such as an antibody specific for a surface membrane protein on the target cell or a ligand for a receptor on the target cell may be incorporated into or attached to the nucleic acid carrier. Preferred antibodies comprise antibodies which bind selectively a tumor-associated antigen. If administration of a nucleic acid via liposomes is desired, proteins binding to a surface membrane protein associated with endocytosis may be incorporated into the liposome formulation in order to make target control and/or uptake possible. Such proteins comprise capsid proteins or fragments thereof which are specific for a particular cell type, antibodies to proteins which are internalized, proteins addressing an intracellular site, and the like.

The therapeutic compositions of the invention may be administered in pharmaceutically compatible preparations. Such preparations may usually contain pharmaceutically compatible concentrations of salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds.

The therapeutically active compounds of the invention may be administered via any conventional route, including by injection or infusion. The administration may be carried out, for example, orally, intravenously, intraperitonealy, intramuscularly, subcutaneously or transdermally. Preferably, antibodies are therapeutically administered by way of a lung aerosol. Antisense nucleic acids are preferably administered by slow intravenous administration.

The compositions of the invention are administered in effective amounts. An “effective amount” refers to the amount which achieves a desired reaction or a desired effect alone or together with further doses. In the case of treatment of a particular disease or of a particular condition characterized by expression of one or more tumor-associated antigens, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. According to the invention, a diagnosis or treatment of cancer may also include the diagnosis or treatment of cancer metastases which have already formed or will form. According to the invention, the term “treatment” comprises therapeutic and prophylactic treatment, i.e. prevention.

An effective amount of a composition of the invention will depend on the condition to be treated, the severeness of the disease, the individual parameters of the patient, including age, physiological condition, size and weight, the duration of treatment, the type of an accompanying therapy (if present), the specific route of administration and similar factors.

The pharmaceutical compositions of the invention are preferably sterile and contain an effective amount of the therapeutically active substance to generate the desired reaction or the desired effect.

The doses administered of the compositions of the invention may depend on various parameters such as the type of administration, the condition of the patient, the desired period of administration, etc. In the case that a reaction in a patient is insufficient with an initial dose, higher doses (or effectively higher doses achieved by a different, more localized route of administration) may be used.

Generally, doses of the tumor-associated antigen of from 1 ng to 1 mg, preferably from 10 ng to 100 μg, are formulated and administered for a treatment or for generating or increasing an immune response. If the administration of nucleic acids (DNA and RNA) coding for tumor-associated antigens is desired, doses of from 1 ng to 0.1 mg are formulated and administered.

The pharmaceutical compositions of the invention are generally administered in pharmaceutically compatible amounts and in pharmaceutically compatible compositions. The term “pharmaceutically compatible” refers to a nontoxic material which does not interact with the action of the active component of the pharmaceutical composition. Preparations of this kind may usually contain salts, buffer substances, preservatives, carriers and, where appropriate, other therapeutically active compounds. When used in medicine, the salts should be pharmaceutically compatible. However, salts which are not pharmaceutically compatible may used for preparing pharmaceutically compatible salts and are included in the invention. Pharmacologically and pharmaceutically compatible salts of this kind comprise in a nonlimiting way those prepared from the following acids: hydrochloric, hydrobromic, sulfuric, nitric, phosphoric, maleic, acetic, salicylic, citric, formic, malonic, succinic acids, and the like. Pharmaceutically compatible salts may also be prepared as alkali metal salts or alkaline earth metal salts, such as sodium salts, potassium salts or calcium salts.

A pharmaceutical composition of the invention may comprise a pharmaceutically compatible carrier. According to the invention, the term “pharmaceutically compatible carrier” refers to one or more compatible solid or liquid fillers, diluents or encapsulating substances, which are suitable for administration to humans. The term “carrier” refers to an organic or inorganic component, of a natural or synthetic nature, in which the active component is combined in order to facilitate application. The components of the pharmaceutical composition of the invention are usually such that no interaction occurs which substantially impairs the desired pharmaceutical efficacy.

The pharmaceutical compositions of the invention may contain suitable buffer substances such as acetic acid in a salt, citric acid in a salt, boric acid in a salt and phosphoric acid in a salt.

The pharmaceutical compositions may, where appropriate, also contain suitable preservatives such as benzalkonium chloride, chlorobutanol, paraben and thimerosal.

The pharmaceutical compositions are usually provided in a uniform dosage form and may be prepared in a manner known per se. Pharmaceutical compositions of the invention may be in the form of capsules, tablets, lozenges, solutions, suspensions, syrups, elixirs or in the form of an emulsion, for example.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

The present invention is described in detail by the figures and examples below, which are used only for illustration purposes and are not meant to be limiting. Owing to the description and the examples, further embodiments which are likewise included in the invention are accessible to the skilled worker.

### FIGURES

FIG. 1. Expression of a tumor-associated nucleic acid identified according to the present invention in normal tissues and cancer tissue Significant expression of the nucleic acid sequence according to SEQ ID NO:540 was found only in placenta tissue and mamma carcinomas.

FIG. 2. Quantitative expression of a tumor-associated nucleic acid identified according to the present invention in normal tissues and cancer tissue Quantitative RT-PCR showed selective expression of the nucleic acid sequence according to SEQ ID NO:540 in placenta tissue and mamma carcinomas.

FIG. 3. Quantitative expression of SEQ ID NO:540 mRNA in MCF-7 breast cancer cells Real-time RT-PCR 24 h after transfection with siRNA oligos showed that both SEQ ID NO:540-specific siRNAs (siRNA#1 (SEQ ID NO:630, 631), siRNA#2 (SEQ ID NO:632, 633)) induce robust silencing of SEQ ID NO:540 expression.

FIG. 4. Silencing of SEQ ID NO:540 expression by transfection with siRNA oligos results in impaired proliferation of MCF-7 breast cancer cells Proliferation was quantified 96 h after transfection with siRNAs by measuring incorporation of BrdU in newly synthesized DNA strands. These results show that SEQ ID NO:540 is a positive factor for the proliferation of breast cancer cells.

FIG. 5. Quantitative expression of SEQ ID NO:541 in normal tissues and cancer tissue Real-time RT-PCR showed overexpression of the nucleic acid sequence according to SEQ ID NO:541 in lung cancer.

FIG. 6. Quantitative expression of SEQ ID NO:545 in normal tissues and cancer tissues Real-time RT-PCR showed overexpression of the nucleic acid sequence according to SEQ ID NO:545 in malignant melanomas.

FIG. 7. Quantitative expression of SEQ ID NO:549 in normal tissues and cancer tissues Real-time RT-PCR showed overexpression of the nucleic acid sequence according to SEQ ID NO:549 in ovarian cancer.

FIG. 8. Quantitative expression of SEQ ID NO:553 in normal tissues and cancer tissues Real-time RT-PCR showed overexpression of the nucleic acid sequence according to SEQ ID NO:553 in colon cancer and ovarian cancer.

FIG. 9. Quantitative expression of SEQ ID NO:557 in normal tissues and cancer tissues Real-time RT-PCR showed overexpression of the nucleic acid sequence according to SEQ ID NO:557 in breast cancer.

FIG. 10. Quantitative expression of SEQ ID NO:560 in normal tissues and cancer tissues Real-time RT-PCR showed overexpression of the nucleic acid sequence according to SEQ ID NO:560 in colon cancer and ovarian cancer.

FIG. 11. Quantitative expression of SEQ ID NO:563 in normal tissues and cancer tissues Real-time RT-PCR showed overexpression of the nucleic acid sequence according to SEQ ID NO:563 in breast cancer, colon cancer, ovarian cancer, lung cancer and melanoma.

FIG. 12. Quantitative expression of SEQ ID NO:566 in normal tissues and cancer tissues Real-time RT-PCR showed overexpression of the nucleic acid sequence according to SEQ ID NO:566 in gastric cancer, breast cancer, colon cancer, ovarian cancer, lung cancer and melanoma.

FIG. 13. Quantitative expression of SEQ ID NO:570 in normal tissues and cancer tissues Real-time RT-PCR showed overexpression of the nucleic acid sequence according to SEQ ID NO:570 in ovarian cancer, lung cancer and melanoma.

FIG. 14. Quantitative expression of SEQ ID NO:574 in normal tissues and cancer tissues Real-time RT-PCR showed overexpression of the nucleic acid sequence according to SEQ ID NO:574 in lung cancer and melanoma.

FIG. 15. Quantitative expression of SEQ ID NO:577 in normal tissues and cancer tissues Real-time RT-PCR showed overexpression of the nucleic acid sequence according to SEQ ID NO:577 in gastric cancer, breast cancer and lung cancer.

FIG. 16. Quantitative expression of SEQ ID NO:580 in normal tissues and cancer tissues Real-time RT-PCR showed overexpression of the nucleic acid sequence according to SEQ ID NO:580 in ovarian cancer and lung cancer.

FIG. 17. Quantitative expression of SEQ ID NO:583 in normal tissues and cancer tissues Real-time RT-PCR showed overexpression of the nucleic acid sequence according to SEQ ID NO:583 in colon cancer, ovarian cancer and lung cancer.

FIG. 18. Quantitative expression of SEQ ID NO:587 in normal tissues and cancer tissues Real-time RT-PCR showed overexpression of the nucleic acid sequence according to SEQ ID NO:587 in lung cancer.

FIG. 19. Quantitative expression of SEQ ID NO:591 in normal tissues and cancer tissues Real-time RT-PCR showed overexpression of the nucleic acid sequence according to SEQ ID NO:591 in breast cancer, colon cancer, ovarian cancer, lung cancer and melanoma.

FIG. 20. Quantitative expression of SEQ ID NO:595 in normal tissues and cancer tissues Real-time RT-PCR showed overexpression of the nucleic acid sequence according to SEQ ID NO:595 in gastric cancer, colon cancer, ovarian cancer, lung cancer and melanoma.

FIG. 21. Quantitative expression of SEQ ID NO:599 in normal tissues and cancer tissues Real-time RT-PCR showed overexpression of the nucleic acid sequence according to SEQ ID NO:599 in gastric cancer, breast cancer, lung cancer and melanoma.

FIG. 22. Quantitative expression of SEQ ID NO:602 in normal tissues and cancer tissues Real-time RT-PCR showed overexpression of the nucleic acid sequence according to SEQ ID NO:602 in ovarian cancer and lung cancer.

FIG. 23. Quantitative expression of SEQ ID NO:606 in normal tissues and cancer tissues Real-time RT-PCR showed overexpression of the nucleic acid sequence according to SEQ ID NO:606 in gastric cancer, colon cancer and lung cancer.

FIG. 24. Quantitative expression of SEQ ID NO:610 in normal tissues and cancer tissues Real-time RT-PCR showed overexpression of the nucleic acid sequence according to SEQ ID NO:610 in gastric cancer, breast cancer and lung cancer.

FIG. 25. Quantitative expression of SEQ ID NO:613 in normal tissues and cancer tissues Real-time RT-PCR showed overexpression of the nucleic acid sequence according to SEQ ID NO:613 in breast cancer, lung cancer and melanoma.

FIG. 26. Quantitative expression of SEQ ID NO:617 in normal tissues and cancer tissues Real-time RT-PCR showed overexpression of the nucleic acid sequence according to SEQ ID NO:617 in lung cancer and melanoma.

FIG. 27. Quantitative expression of SEQ ID NO:620 in normal tissues and cancer tissues Real-time RT-PCR showed overexpression of the nucleic acid sequence according to SEQ ID NO:620 in ovarian cancer and melanoma.

FIG. 28. Quantitative expression of SEQ ID NO:624 in normal tissues and cancer tissues Real-time RT-PCR showed overexpression of the nucleic acid sequence according to SEQ ID NO:624 in gastric cancer and lung cancer.

FIG. 29. Quantitative expression of SEQ ID NO:634 in normal tissues and cancer tissues Real-time RT-PCR showed overexpression of the nucleic acid sequence according to SEQ ID NO:634 in ovarian cancer and lung cancer.

FIG. 30. Quantitative expression of SEQ ID NO:638 in normal tissues and cancer tissues Real-time RT-PCR showed overexpression of the nucleic acid sequence according to SEQ ID NO:638 in colon cancer, ovarian cancer, lung cancer, and malignant melanomas.

FIG. 31. Quantitative expression of SEQ ID NO:642 in normal tissues and cancer tissues Real-time RT-PCR showed overexpression of the nucleic acid sequence according to SEQ ID NO:642 in breast cancer, ovarian cancer, lung cancer, and malignant melanomas.

FIG. 32. Quantitative expression of SEQ ID NO:646 in normal tissues and cancer tissues Real-time RT-PCR showed overexpression of the nucleic acid sequence according to SEQ ID NO:646 in gastric cancer, ovarian cancer, and lung cancer.

FIG. 33. Quantitative expression of SEQ ID NO:649 in normal tissues and cancer tissues Real-time RT-PCR showed overexpression of the nucleic acid sequence according to SEQ ID NO:649 in ovarian cancer, lung cancer, and malignant melanomas.

FIG. 34. Quantitative expression of SEQ ID NO:653 in normal tissues and cancer tissues Real-time RT-PCR showed overexpression of the nucleic acid sequence according to SEQ ID NO:653 in colon cancer, and lung cancer.

FIG. 35. Quantitative expression of SEQ ID NO:656 in normal tissues and cancer tissues Real-time RT-PCR showed overexpression of the nucleic acid sequence according to SEQ ID NO:656 in lung cancer, and malignant melanomas.

FIG. 36. Quantitative expression of SEQ ID NO:660 in normal tissues and cancer tissues Real-time RT-PCR showed overexpression of the nucleic acid sequence according to SEQ ID NO:660 in gastric cancer, ovarian cancer, and malignant melanomas.

FIG. 37. Quantitative expression of SEQ ID NO:664 in normal tissues and cancer tissues Real-time RT-PCR showed overexpression of the nucleic acid sequence according to SEQ ID NO:664 in gastric cancer, colon cancer, lung cancer, and malignant melanomas.

FIG. 38. Quantitative expression of SEQ ID NO:668 in normal tissues and cancer tissues Real-time RT-PCR showed overexpression of the nucleic acid sequence according to SEQ ID NO:668 in lung cancer.

FIG. 39. Quantitative expression of SEQ ID NO:671 in normal tissues and cancer tissues Real-time RT-PCR showed overexpression of the nucleic acid sequence according to SEQ ID NO:671 in lung cancer, and malignant melanomas.

FIG. 40. Quantitative expression of SEQ ID NO:675 in normal tissues and cancer tissues Real-time RT-PCR showed overexpression of the nucleic acid sequence according to SEQ ID NO:675 in lung cancer, and malignant melanomas.

FIG. 41. Quantitative expression of SEQ ID NO:679 in normal tissues and cancer tissues Real-time RT-PCR showed overexpression of the nucleic acid sequence according to SEQ ID NO:679 in lung cancer.

FIG. 42. Quantitative expression of SEQ ID NO:682 in normal tissues and cancer tissues Real-time RT-PCR showed overexpression of the nucleic acid sequence according to SEQ ID NO:682 in gastric cancer, breast cancer, and malignant melanomas.

FIG. 43. Quantitative expression of SEQ ID NO:686 in normal tissues and cancer tissues Real-time RT-PCR showed overexpression of the nucleic acid sequence according to SEQ ID NO:686 in breast cancer, ovarian cancer, and lung cancer.

### EXAMPLES

The techniques and methods mentioned herein are carried out in a manner known per se and are described, for example, in Sambrook et al., Molecular Cloning: A Laboratory Manual, 2nd Edition (1989) Cold Spring Harbor Laboratory Press, Cold Spring Harbor, N.Y. All methods including the use of kits and reagents are carried out according to the manufacturers' information unless specifically indicated.

### Example 1

### Screening for Placenta-Specific Genes Aberrantly Activated in Tumors

### Tissues and Cell Lines

Tissues were obtained as human surplus materials during routine diagnostic or therapeutic procedures and were stored at −80° C. until use. Cell lines were purchased from the American Type Culture Collection (ATCC) and the German Resource Collection of Microorganisms and Cell Culture (DSMZ).

### RNA Isolation and Microarray Hybridization

Total RNA was isolated using the RNeasy Mini Kit protocol (Qiagen).

Quantification of isolated RNA was performed using UV-spectroscopy and the quality was determined both by A₂₆₀/A₂₈₀ ratio and Agilent bioanalyzer (Agilent Technologies). Five micrograms total RNA were used for cDNA synthesis with 5 pmol μl⁻¹ T7-oligo(dT)₂₄ primer and was performed at 43° C. for 90 minutes with the “Superscript First-Strand Synthesis-System” for RT-PCR (Invitrogen). Second-strand synthesis was performed with complete cDNA. The cDNA solution was incubated at 16° C. for 2 hours followed by an incubation step for 20 min with 6 U T4-DNA polymerase at 16° C. and the reaction was stopped using 10 μl of 0.5 M EDTA. After purification of the double stranded cDNA using the GeneChip Sample Cleanup Module (Affymetrix) labeled cRNA was generated from the cDNA sample by an in vitro transcription reaction that was supplemented with biotin-11-CTP and biotin-16-UTP (Enzo Diagnostics) according to the manufacturer's instructions. The cRNA was quantified by A₂₆₀, and the quality was determined using the labchip bioanalyzer (Agilent). Only cRNA specimens with a high quality were selected for further analyses. Fragmented cRNA (15 μg) was used to prepare 300 μl hybridization cocktail (100 mM MES, 1 M NaCl, 20 mM EDTA, 0.01% Tween-20) containing 0.1 mg ml⁻¹ of herring sperm DNA, and 0.5 mg ml⁻¹ acetylated bovine serum albumine. Control cRNA was used in order to compare hybridization efficiencies between arrays and to standardize the quantification of measured transcript levels and was included as component of the ‘Eukaryotic Hybridization Control kit’ (Affymetrix, Santa Clara, Calif., USA). The cocktails were heated to 95° C. for 5 minutes, equilibrated at 45° C. for 5 minutes, and clarified by centrifugation. The cocktail was hybridized to HG U133 Plus 2.0 arrays (Affymetrix) at 45° C. for 16 hours. The arrays were washed and stained with a streptavidin-conjugated fluor using the GeneChip fluidics station protocol EukGE-WS2 (Affymetrix) according to the manufacturer's instructions. Arrays were scanned with an argon-ion laser confocal scanner (Hewlett-Packard, Santa Clara, Calif.) with detection at 570 nm. Data were extracted using Microarray Suite version 5.0 (Affymetrix) and linearly scaled to achieve an average intensity of 2,500 per gene. Text files were exported to determine the intensity of each interrogating oligonucleotide perfect match probe cells or mismatch probe cells. In addition, the ratios of 5′- and 3′-ends of mRNA were analyzed of six randomly selected specimens (two of each group) using microarray test-chips (Test3 Array) containing 24 human housekeeping/maintenance genes (Affymetrix) and RNA degradation was not observed.

### Bioinformatic Analysis

The GeneChip® Operating Software 1.4 (Affymetrix) and ArrayAssist software package 5.2 (Stratagene) were used for statistical analyses.

### Results

Screening of samples from the 18 normal tissues shown below in table 1 and 30 tumor cell lines of different entities shown below in table 2 resulted in the sequences described herein which are expressed in placenta among the normal tissues investigated and in tumor cell lines.

TABLE 1
Tissues used for microarray expression analysis

| Tissue | Number |
|---|---|
| Placenta | 2 |
| Testis | 2 |
| Mammary gland | 2 |
| Thymus | 2 |
| Skin | 2 |
| Liver | 2 |
| Colon | 2 |
| Esophagus | 2 |
| Stomach | 2 |
| Lung | 2 |
| Kidney | 2 |
| Lymph node | 2 |
| Skeletal muscle | 2 |
| Myocard | 1 |
| Brain | 1 |
| Cerebellum | 1 |
| resting PBMCs | 2 |
| activ. PBMCs | 2 |

TABLE 2
Cell lines used for microarray expression analysis

| Cell line | Tissue |
|---|---|
| BT-549 | Breast cancer |
| MDA-MB-231 metastasizing | Breast cancer |
| MDA-MB-231 non-metastatsizing | Breast cancer |
| MDA-MB-435S | Breast cancer |
| MDA-MB-468 | Breast cancer |
| SK-BR-3 | Breast cancer |
| Caov-3 | Ovarian cancer |
| FU-OV | Ovarian cancer |
| NIH-OVCAR-3 | Ovarian cancer |
| COLO-205 | Colorectal cancer |
| HCT-116 | Colorectal cancer |
| HCT-116 DKO | Colorectal cancer |
| HCT-15 | Colorectal cancer |
| HT-29 | Colorectal cancer |
| LOVO | Colorectal cancer |
| SW-480 | Colorectal cancer |
| CPC-N | Lung cancer |
| LOU-NH-91 | Lung cancer |
| SHP-77 | Lung cancer |
| SK-MES-1 | Lung cancer |
| NCI-H-187 | Lung cancer |
| NCI-H-209 | Lung cancer |
| NCI-H-522 | Lung cancer |
| DU-145 | Prostate cancer |
| LnCaP | Prostate cancer |
| PC-3 | Prostate cancer |
| MEL-JUSO | Melanoma |
| Murowsky | Melanoma |
| SK-MEL-37 | Melanoma |
| HELA | Cervical cancer |

### Example 2

### Validation of the Identified Tumor-Associated Markers

### 1. Examination of RNA Expression

The identified tumor-associated markers are first validated with the aid of RNA which is obtained from various tissues or from tissue-specific cell lines. Since the differential expression pattern of healthy tissue in comparison with tumor tissue is of decisive importance for the subsequent therapeutic application, the target genes are preferably characterized with the aid of these tissue samples.

Total RNA is isolated from native tissue samples or from tumor cell lines by standard methods of molecular biology. Said isolation may be carried out, for example, with the aid of the RNeasy Maxi kit (Qiagen, Cat. No. 75162) according to the manufacturer's instructions. This isolation method is based on the use of chaotropic reagent guanidinium isothiocyanate. Alternatively, acidic phenol can be used for isolation (Chomczynski & Sacchi, Anal. Biochem. 162: 156-159, 1987). After the tissue has been worked up by means of guanidinium isothiocyanate, RNA is extracted with acidic phenol, subsequently precipitated with isopropanol and taken up in DEPC-treated water.

2-4 μg, of the RNA isolated in this way are subsequently transcribed into cDNA, for example by means of Superscript II (Invitrogen) according to the manufacturer's protocol. cDNA synthesis is primed with the aid of random hexamers (e.g. Roche Diagnostics) according to standard protocols of the relevant manufacturer. For quality control, the cDNAs are amplified over 30 cycles, using primers specific for the p53 gene which is expressed only lowly. Only p53-positive cDNA samples will be used for the subsequent reaction steps.

The targets are analyzed in detail by carrying out an expression analysis by means of PCR or quantitative PCR (qPCR) on the basis of a cDNA archive which has been isolated from various normal and tumor tissues and from tumor cell lines. For this purpose, 0.5 μl of cDNA of the above reaction mixture is amplified by a DNA polymerase (e.g. 1 U of HotStarTaq DNA polymerase, Qiagen) according to the protocols of the particular manufacturer (total volume of the reaction mixture: 25-50 μl). Aside from said polymerase, the amplification mixture comprises 0.3 mM dNTPs, reaction buffer (final concentration 1×, depending on the manufacturer of the DNA polymerase) and in each case 0.3 mM gene-specific “sense” and “antisense” primers.

The specific primers of the target gene are, as far as possible, selected in such a way that they are located in two different exons so that genomic contaminations do not lead to false-positive results. In a non-quantitative end point PCR, the cDNA is typically incubated at 95° C. for 15 minutes in order to denature the DNA and to activate the Hot-Start enzyme. Subsequently the DNA is amplified over 35 cycles (1 min at 95° C., 1 min at the primer-specific hybridization temperature (approx. 55-65° C.), 1 min at 72° C. to elongate the amplicons). Subsequently, 10 μl of the PCR mixture are applied to agarose gels and fractionated in the electric field. The DNA is made visible in the gels by staining with ethidium bromide and the PCR result is documented by way of a photograph.

As an alternative to conventional PCR, expression of a target gene may also be analyzed by quantitative real time PCR. Meanwhile various analytical systems are available for this analysis, of which the best known ones are the ABI PRISM sequence detection system (TaqMan, Applied Biosystems), the iCycler (Biorad) and the Light cycler (Roche Diagnostics). As described above, a specific PCR mixture is subjected to a run in the real time instruments. By adding a DNA-intercalating dye (e.g. ethidium bromide, CybrGreen), the newly synthesized DNA is made visible by specific light excitation (according to the dye manufacturers' information). A multiplicity of points measured during amplification enables the entire process to be monitored and the nucleic acid concentration of the target gene to be determined quantitatively. The PCR mixture is normalized by measuring a housekeeping gene (e.g. 18S RNA, β-actin). Alternative strategies via fluorescently labeled DNA probes likewise allow quantitative determination of the target gene of a specific tissue sample (see TaqMan applications from Applied Biosystems).

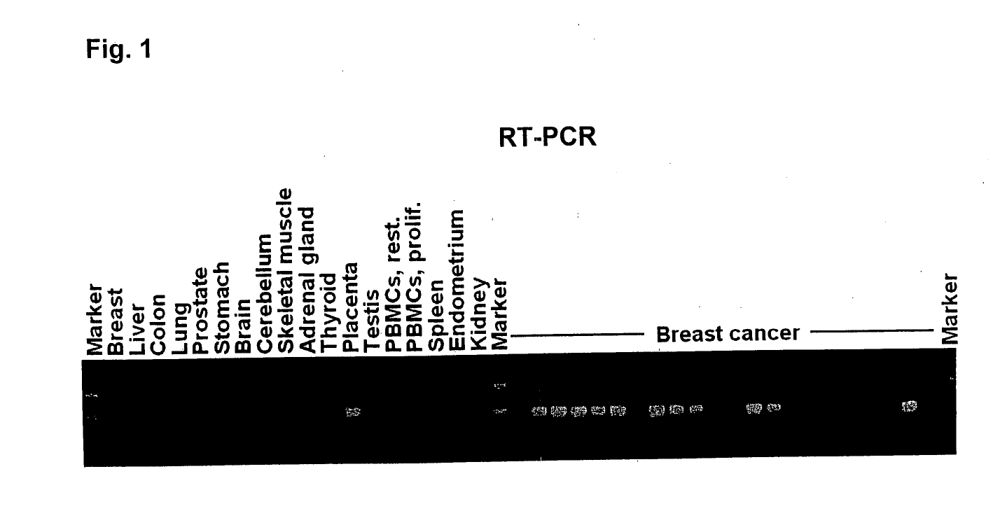

As shown in FIG. 1, placenta was confirmed in RT-PCR analyses as the only healthy tissue expressing the nucleic acid sequence according to SEQ ID NO:540. No significant expression was found in any other normal tissue. However, high and significant levels of expression were found in breast cancer.

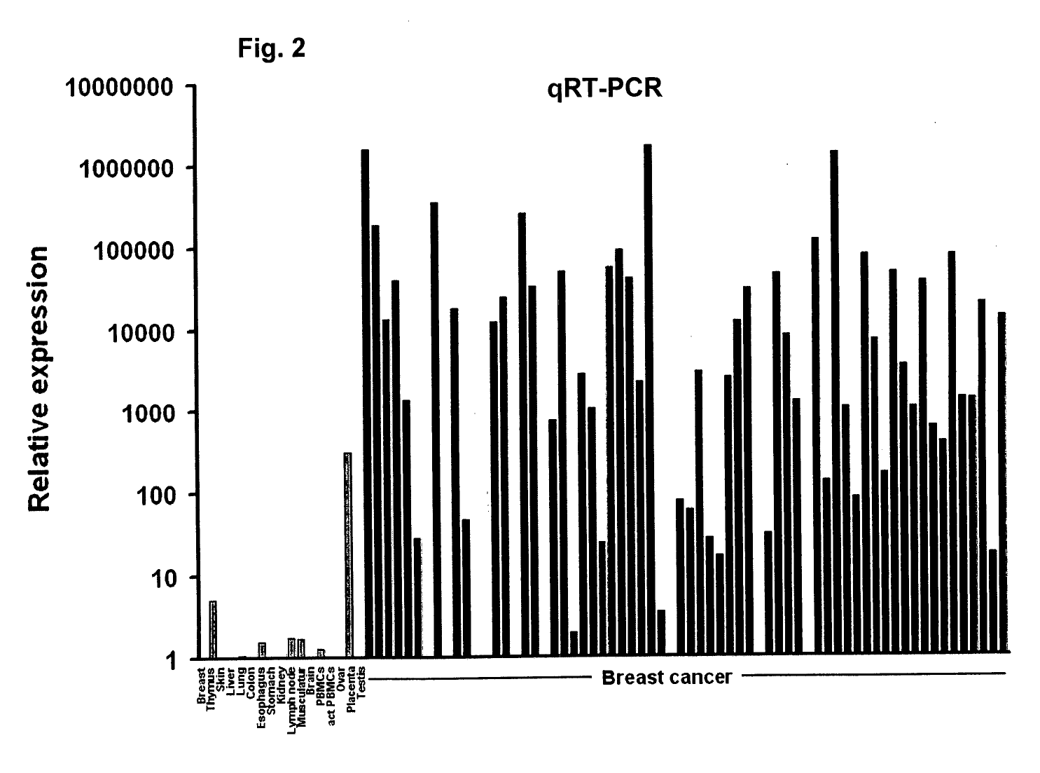

Quantitative real-time RT-PCR analyses revealed that the nucleic acid sequence according to SEQ ID NO:540 was expressed in significant levels in the majority of breast cancer samples analyzed; cf. FIG. 2.

### 2. Cloning

The complete target gene which is required for further characterization of the tumor-associated marker is cloned according to common molecular-biological methods (e.g. in “Current Protocols in Molecular Biology”, John Wiley & Sons Ltd., Wiley InterScience). In order to clone the target gene or to analyze its sequence, said gene is first amplified by a DNA polymerase having a proof reading function (e.g. pfu, Roche Diagnostics). The amplicon is then ligated by standard methods into a cloning vector. Positive clones are identified by sequence analysis and subsequently characterized with the aid of prediction programs and known algorithms.

### 3. Prediction of the Protein

Genes found according to the invention (in particular those from the RefSeq XM domain) may require cloning of the full-length gene, determination of the open reading frame and deduction and analysis of the protein sequence.

In order to clone the full-length sequence, common protocols for the rapid amplification of cDNA ends and the screening of cDNA expression libraries with gene-specific probes may be used (Sambrook et al., Molecular Cloning: A Laboratory Manual, 2nd edition (1989), Cold Spring Harbor Laboratory Press, Cold Spring Harbor, N.Y.).

After assembling the fragments found in this way, potential open reading frames (ORF) can be predicted using common prediction programs. Since the position of the PolyA tail and of polyadenylation motifs predetermines the orientation of the potential gene product, only the 3 reading frames of that particular orientation remain out of a possible 6 reading frames. The former often yield only one sufficiently large open reading frame which may code for a protein, while the other reading frames have too many stop codons and would not code for any realistic protein. In the case of alternative open reading frames, identification of the authentic ORF is assisted by taking into account the Kozak criteria for optimal transcription initiation and by analyzing the deduced protein sequences which may arise. Said ORF is further verified by generating immune sera against proteins deduced from the potential ORFs and analyzing said immune sera for recognition of a real protein in tissues and cell lines.

### 4. Production of Antibodies

The tumor-associated antigens identified according to the invention are characterized, for example, by using antibodies. The invention further comprises the diagnostic or therapeutic use of antibodies. Antibodies may recognize proteins in the native and/or denatured state (Anderson et al., J. Immunol. 143: 1899-1904, 1989; Gardsvoll, J. Immunol. Methods 234: 107-116, 2000; Kayyem et al., Eur. J. Biochem. 208: 1-8, 1992; Spiller et al., J. Immunol. Methods 224: 51-60, 1999).

Antisera comprising specific antibodies which specifically bind to the target protein may be prepared by various standard methods; cf., for example, “Monoclonal Antibodies: A Practical Approach” by Phillip Shepherd, Christopher Dean ISBN 0-19-963722-9, “Antibodies: A Laboratory Manual” by Ed Harlow, David Lane ISBN: 0879693142 and “Using Antibodies: A Laboratory Manual: Portable Protocol NO” by Edward Harlow, David Lane, Ed Harlow ISBN: 0879695447. It is also possible here to generate affine and specific antibodies which recognize complex membrane proteins in their native form (Azorsa et al., J. Immunol. Methods 229: 35-48, 1999; Anderson et al., J. Immunol. 143: 1899-1904, 1989; Gardsvoll, J. Immunol. Methods. 234: 107-116, 2000). This is especially important in the preparation of antibodies which are intended to be used therapeutically but also for many diagnostic applications. For this purpose, both the complete protein and extracellular partial sequences may be used for immunization.

### Immunization and Production of Polyclonal Antibodies

Various immunization protocols are published. A species (e.g. rabbits, mice) is immunized by a first injection of the desired target protein. The immune response of the animal to the immunogen can be enhanced by a second or third immunization within a defined period of time (approx. 2-4 weeks after the previous immunization). Blood is taken from said animals and immune sera obtained, again after various defined time intervals (1st bleeding after 4 weeks, then every 2-3 weeks, up to 5 takings). The immune sera taken in this way comprise polyclonal antibodies which may be used to detect and characterize the target protein in Western blotting, by flow cytometry, immunofluorescence or immunohistochemistry.

The animals are usually immunized by any of four well-established methods, with other methods also in existence. The immunization may be carried out using peptides specific for the target protein, using the complete protein, or using extracellular partial sequences of a protein which can be identified experimentally or via prediction programs. Since the prediction programs do not always work perfectly, it is also possible to employ two domains separated from one another by a transmembrane domain. In this case, one of the two domains has to be extracellular, which may then be proved experimentally (see below). Immunization is offered commercially by different service providers.
(1) In the first case, peptides (length: 8-12 amino acids) are synthesized by in vitro methods (possibly carried out by a commercial service), and said peptides are used for immunization. Normally 3 immunizations are carried out (e.g. with a concentration of 5-100 μg/immunization).
(2) Alternatively, immunization may be carried out using recombinant proteins. For this purpose, the cloned DNA of the target gene is cloned into an expression vector and the target protein is synthesized, for example, cell-free in vitro, in bacteria (e.g. E. coli), in yeast (e.g. S. pombe), in insect cells or in mammalian cells, according to the conditions of the particular manufacturer (e.g. Roche Diagnostics, Invitrogen, Clontech, Qiagen). It is also possible to synthesize the target protein with the aid of viral expression systems (e.g. baculovirus, vacciniavirus, adenovirus). After it has been synthesized in one of said systems, the target protein is purified, normally by employing chromatographic methods. In this context, it is also possible to use for immunization proteins which have a molecular anchor as an aid for purification (e.g. His tag, Qiagen; FLAG tag, Roche Diagnostics; GST fusion proteins). A multiplicity of protocols can be found, for example, in “Current Protocols in Molecular Biology”, John Wiley & Sons Ltd., Wiley InterScience. After the target protein has been purified, an immunization is carried out as described above.
(3) If a cell line is available which synthesizes the desired protein endogenously, it is also possible to use this cell line directly for preparing the specific antiserum. In this case, immunization is carried out by 1-3 injections with in each case approx. 1−5×10⁷ cells.
(4) The immunization may also be carried out by injecting DNA (DNA immunization). For this purpose, the target gene is first cloned into an expression vector so that the target sequence is under the control of a strong eukaryotic promoter (e.g. CMV promoter). Subsequently, DNA (e.g. 1-10 μg per injection) is transferred as immunogen using a gene gun into capillary regions with a strong blood flow in an organism (e.g. mouse, rabbit). The transferred DNA is taken up by the animal's cells, the target gene is expressed, and the animal finally develops an immune response to the target protein (Jung et al., Mol. Cells 12: 41-49, 2001; Kasinrerk et al., Hybrid Hybridomics 21: 287-293, 2002).

### Production of Monoclonal Antibodies

Monoclonal antibodies are traditionally produced with the aid of the hybridoma technology (technical details: see “Monoclonal Antibodies: A Practical Approach” by Philip Shepherd, Christopher Dean ISBN 0-19-963722-9; “Antibodies: A Laboratory Manual” by Ed Harlow, David Lane ISBN: 0879693142, “Using Antibodies: A Laboratory Manual: Portable Protocol NO” by Edward Harlow, David Lane, Ed Harlow ISBN: 0879695447). A new method which is also used is the “SLAM” technology. Here, B cells are isolated from whole blood and the cells are made monoclonal. Subsequently the supernatant of the isolated B cell is analyzed for its antibody specificity. In contrast to the hybridoma technology, the variable region of the antibody gene is then amplified by single-cell PCR and cloned into a suitable vector. In this manner production of monoclonal antibodies is accelerated (de Wildt et al., J. Immunol. Methods 207:61-67, 1997).

### 5. Validation of the Targets by Protein-Chemical Methods Using Antibodies

The antibodies which can be produced as described above can be used to further analyse the target protein as follows:

### Specificity of the Antibody

Assays based on cell culture with subsequent Western blotting are most suitable for demonstrating the fact that an antibody binds specifically only to the desired target protein (various variations are described, for example, in “Current Protocols in Proteinchemistry”, John Wiley & Sons Ltd., Wiley InterScience). For the demonstration, cells are transfected with a cDNA for the target protein, which is under the control of a strong eukaryotic promoter (e.g. cytomegalovirus promoter; CMV). A wide variety of methods (e.g. electroporation, liposome-based transfection, calcium phosphate precipitation) are well established for transfecting cell lines with DNA (e.g. Lemoine et al., Methods Mol. Biol. 75: 441-7, 1997). As an alternative, it is also possible to use cell lines which express the target gene endogenously (detection via target gene-specific RT-PCR). As a control, in the ideal case, homologous genes are cotransfected in the experiment, in order to be able to demonstrate in the following Western blot the specificity of the analyzed antibody.

In the subsequent Western blotting, cells from cell culture or tissue samples which might contain the target protein are lysed in a 1% strength SDS solution, and the proteins are denatured in the process. The lysates are fractionated according to size by electrophoresis on 8-15% strength denaturing polyacrylamide gels (contain 1% SDS) (SDS polyacrylamide gel electrophoresis, SDS-PAGE). The proteins are then transferred by one of a plurality of blotting methods (e.g. semi-dry electroblot; Biorad) to a specific membrane (e.g. nitrocellulose, Schleicher & Schüll). The desired protein can be visualized on this membrane. For this purpose, the membrane is first incubated with the antibody which recognizes the target protein (dilution approx. 1:20-1:200, depending on the specificity of said antibody), for 60 minutes. After a washing step, the membrane is incubated with a second antibody which is coupled to a marker (e.g. enzymes such as peroxidase or alkaline phosphatase) and which recognizes the first antibody. It is then possible to make the target protein visible on the membrane in a color or chemiluminescent reaction (e.g. ECL, Amersham Bioscience). An antibody with a high specificity for the target protein should in the ideal case only recognise the desired protein itself.

### Localization of the Target Protein

Various methods are used to confirm the membrane localization, identified in the in silico approach, of the target protein. An important and well-established method using the antibodies described above is immunofluorescence (IF). For this purpose, cells of established cell lines which either synthesize the target protein (detection of the RNA by RT-PCR or of the protein by Western blotting) or else have been transfected with plasmid DNA are utilized. A wide variety of methods (e.g. electroporation, liposome-based transfection, calcium phosphate precipitation) are well established for transfection of cell lines with DNA (e.g. Lemoine et al., Methods Mol. Biol. 75: 441-7, 1997). The plasmid transfected into the cells, in immunofluorescence, may encode the unmodified protein or else couple different amino acid markers to the target protein. The principle markers are, for example, the fluorescent green fluorescent protein (GFP) in various differentially fluorescent forms, short peptide sequences of 6-12 amino acids for which high-affinity and specific antibodies are available, or the short amino acid sequence Cys-Cys-X-X-Cys-Cys which can bind via its cysteines specific fluorescent substances (Invitrogen). Cells which synthesize the target protein are fixed, for example, with paraformaldehyde or methanol. The cells may then, if required, be permeabilized by incubation with detergents (e.g. 0.2% Triton X-100). The cells are then incubated with a primary antibody which is directed against the target protein or against one of the coupled markers. After a washing step, the mixture is incubated with a second antibody coupled to a fluorescent marker (e.g. fluorescein, Texas Red, Dako), which binds to the first antibody. The cells labeled in this way are then overlaid with glycerol and analyzed with the aid of a fluorescence microscope according to the manufacturer's information. Specific fluorescence emissions are achieved in this case by specific excitation depending on the substances employed. The analysis usually permits reliable localization of the target protein, the antibody quality and the target protein being confirmed in double stainings with, in addition to the target protein, also the coupled amino acid markers or other marker proteins whose localization has already been described in the literature being stained. GFP and its derivatives represent a special case, being excitable directly and themselves fluorescing. The membrane permeability which may be controlled through the use of detergents, in immunofluorescence, allows demonstration of whether an immunogenic epitope is located inside or outside the cell. The prediction of the selected proteins can thus be supported experimentally. An alternative possibility is to detect extracellular domains by means of flow cytometry. For this purpose, cells are fixed under non-permeabilizing conditions (e.g. with PBS/Na azide/2% FCS/5 mM EDTA) and analyzed in a flow cytometer in accordance with the manufacturer's instructions. Only extracellular epitopes can be recognized by the antibody to be analyzed in this method. A difference from immunofluorescence is that it is possible to distinguish between dead and living cells by using, for example, propidium iodide or trypan blue, and thus avoid false-positive results.

Another important detection is by immunohistochemistry (IHC) on specific tissue samples. The aim of this method is to identify the localization of a protein in a functionally intact tissue aggregate. IHC serves specifically for (1) being able to estimate the amount of target protein in tumor and normal tissues, (2) analyzing how many cells in tumor and healthy tissues synthesize the target gene, and (3) defining the cell type in a tissue (tumor, healthy cells) in which the target protein is detectable. Alternatively, the amounts of protein of a target gene may be quantified by tissue immunofluorescence using a digital camera and suitable software (e.g. Tillvision, Till-photonics, Germany). The technology has frequently been published, and details of staining and microscopy can therefore be found, for example, in “Diagnostic Immunohistochemistry” by David J., MD Dabbs ISBN: 0443065667 or in “Microscopy, Immunohistochemistry, and Antigen Retrieval Methods: For Light and Electron Microscopy” ISBN: 0306467704. It should be noted that, owing to the properties of antibodies, different protocols have to be used (an example is described below) in order to obtain a meaningful result.

Normally, histologically defined tumor tissues and, as reference, comparable healthy tissues are employed in IHC. It is also possible to use as positive and negative controls cell lines in which the presence of the target gene is known through RT-PCR analyses. A background control must always be included.

Formalin-fixed (another fixation method, for example with methanol, is also possible) and paraffin-embedded tissue pieces with a thickness of 4 μm are applied to a glass support and deparaffinated with xylene, for example. The samples are washed with TBS-T and blocked in serum. This is followed by incubation with the first antibody (dilution: 1:2 to 1:2000) for 1-18 hours, with affinity-purified antibodies normally being used. A washing step is followed by incubation with a second antibody which is coupled to an alkaline phosphatase (alternative: for example peroxidase) and directed against the first antibody, for approx. 30-60 minutes. This is followed by a color reaction using alkaline phosphatase (cf., for example, Shi et al., J. Histochem. Cytochem. 39: 741-748, 1991; Shin et al., Lab. Invest. 64: 693-702, 1991). To demonstrate antibody specificity, the reaction can be blocked by previous addition of the immunogen.

### Analysis of Protein Modifications

Secondary protein modifications such as, for example, N- or O-glycosylations or myristilations may impair or even completely prevent the accessibility of immuno-genic epitopes and thus call into question the efficacy of antibody therapies. Moreover, it has frequently been demonstrated that the type and amount of secondary modifications differ in normal and tumor tissues (e.g. Durand & Seta, 2000; Clin. Chem. 46: 795-805; Hakomori, 1996; Cancer Res. 56: 5309-18). The analysis of these modifications is therefore essential to the therapeutic success of an antibody. Potential binding sites can be predicted by specific algorithms.

Analysis of protein modifications usually takes place by Western blotting (see above). Glycosylations which usually have a size of several kDa, especially lead to a larger total mass of the target protein, which can be fractionated in SDS-PAGE. To detect specific O- and N-glycosidic bonds, protein lysates are incubated prior to denaturation by SDS with O- or N-glycosylases (in accordance with their respective manufacturer's instructions, e.g. PNgase, endoglycosidase F, endo-glycosidase H, Roche Diagnostics). This is followed by Western blotting as described above. Thus, if there is a reduction in the size of a target protein after incubation with a glycosidase, it is possible to detect a specific glycosylation and, in this way, also analyze the tumor specificity of a modification.

### Functional Analysis of the Target Gene

The function of the target molecule may be crucial for its therapeutic usefulness, so that functional analyses are an important component in the characterization of therapeutically utilizable molecules. The functional analysis may take place either in cells in cell culture experiments or else in vivo with the aid of animal models. This involves either switching off the gene of the target molecule by mutation (knockout) or inserting the target sequence into the cell or the organism (knockin). Thus it is possible to analyze functional modifications in a cellular context firstly by way of the loss of function of the gene to be analyzed (loss of function). In the second case, modifications caused by addition of the analyzed gene can be analyzed (gain of function).

a. Functional Analysis in Cells

Transfection. In order to analyze the gain of function, the gene of the target molecule must be transferred into the cell. For this purpose, cells which allow synthesis of the target molecule are transfected with a DNA. Normally, the gene of the target molecule here is under the control of a strong eukaryotic promoter (e.g. cytomegalovirus promoter; CMV). A wide variety of methods (e.g. electroporation, liposome-based transfection, calcium phosphate precipitation) are well established for transfecting cell lines with DNA (e.g. Lemoine et al., Methods Mol. Biol. 75: 441-7, 1997). The gene may be synthesized either transiently, without genomic integration, or else stably, with genomic integration after selection with neomycin, for example.

RNA interference (siRNA). An inhibition of expression of the target gene, which may induce a complete loss of function of the target molecule in cells, may be generated by the RNA interference (siRNA) technology in cells (Hannon, G J. 2002. RNA interference. Nature 418: 244-51; Czauderna et al. 2003. Nucl. Acid Res. 31: 670-82). For this purpose, cells are transfected with short, double-stranded RNA molecules of approx. 20-25 nucleotides in length, which are specific for the target molecule. An enzymic process then results in degradation of the specific RNA of the target gene and thus in reduced expression of the target protein and consequently enables the target gene to be functionally analyzed.

Cell lines which have been modified by means of transfection or siRNA may subsequently be analyzed in different ways. The most common examples are listed below.

### 1. Proliferation and Cell Cycle Behavior

A multiplicity of methods for analyzing cell proliferation are established and are commercially supplied by various companies (e.g. Roche Diagnostics, Invitrogen; details of the assay methods are described in the numerous application protocols). The number of cells in cell culture experiments can be determined by simple counting or by colorimetric assays which measure the metabolic activity of the cells (e.g. wst-1, Roche Diagnostics). Metabolic assay methods measure the number of cells in an experiment indirectly via enzymic markers. Cell proliferation may be measured directly by analyzing the rate of DNA synthesis, for example by adding bromodeoxyuridine (BrdU), with the integrated BrdU being detected colorimetrically via specific antibodies.

### 2. Apoptosis and Cytotoxicity

A large number of assay systems for detecting cellular apoptosis and cytotoxicity are available. A decisive characteristic is the specific, enzyme-dependent fragmentation of genomic DNA, which is irreversible and in any case results in death of the cell. Methods for detecting these specific DNA fragments are commercially obtainable. An additional method available is the TUNEL assay which can detect DNA single-strand breaks also in tissue sections. Cytotoxicity is mainly detected via an altered cell permeability which serves as marker of the vitality state of cells. This involves on the one hand the analysis of markers which can typically be found intracellularly in the cell culture supernatant. On the other hand, it is also possible to analyze the absorbability of dye markers which are not absorbed by intact cells. The best-known examples of dye markers are Trypan blue and propidium iodide, a common intracellular marker is lactate dehydrogenase which can be detected enzymatically in the supernatant. Different assay systems of various commercial suppliers (e.g. Roche Diagnostics, Invitrogen) are available.

### 3. Migration Assay

The ability of cells to migrate is analyzed in a specific migration assay, preferably with the aid of a Boyden chamber (Corning Costar) (Cinamon G., Alon R. J. Immunol. Methods. 2003 February; 273(1-2):53-62; Stockton et al. 2001. Mol. Biol. Cell. 12: 1937-56). For this purpose, cells are cultured on a filter with a specific pore size. Cells which can migrate are capable of migrating through this filter into another culture vessel below. Subsequent microscopic analysis then permits determination of a possibly altered migration behavior induced by the gain of function or loss of function of the target molecule.

b. Functional Analysis in Animal Models

A possible alternative of cell culture experiments for the analysis of target gene function are complicated in vivo experiments in animal models. Compared to the cell-based methods, these models have the advantage of being able to detect faulty developments or diseases which are detectable only in the context of the whole organism. A multiplicity of models for human disorders are available by now (Abate-Shen & Shen. 2002. Trends in Genetics S1-5; Matsusue et al. 2003. J. Clin. Invest. 111:737-47). Various animal models such as, for example, yeast, nematodes or zebra fish have since been characterized intensively. However, models which are preferred over other species are mammalian animal models such as, for example, mice (Mus musculus) because they offer the best possibility of reproducing the biological processes in a human context. For mice, on the one hand transgenic methods which integrate new genes into the mouse genome have been established in recent years (gain of function; Jegstrup I. et al. 2003. Lab Anim. 2003 January; 37(1):1-9). On the other hand, other methodical approaches switch off genes in the mouse genome and thus induce a loss of function of a desired gene (knockout models, loss of function; Zambrowicz B P & Sands A T. 2003. Nat. Rev. Drug Discov. 2003 January; 2(1):38-51; Niwa H.2001. Cell Struct. Funct. 2001 June; 26(3):137-48); technical details have been published in large numbers.

After the mouse models have been generated, alterations induced by the transgene or by the loss of function of a gene can be analyzed in the context of the whole organism (Balling R, 2001. Ann. Rev. Genomics Hum. Genet. 2:463-92). Thus it is possible to carry out, for example, behavior tests as well as to biochemically study established blood parameters. Histological analyses, immunohistochemistry or electron microscopy enable alterations to be characterized at the cellular level. The specific expression pattern of a gene can be detected by in-situ hybridization (Peters T. et al. 2003. Hum. Mol. Genet. 12:2109-20).

### Example 3

### Detailed Analysis of the Identified Tumor-Associated Markers

### RNA-Isolation, RT-PCR and Real-Time RT-PCR

RNA extraction, first-strand cDNA synthesis, RT-PCR and real-time RT-PCR was performed as previously described (Koslowski, M. et al., Cancer Res. 62, 6750-6755 (2002), Koslowski, M. et al., Cancer Res. 64, 5988-5993 (2004)). Real-time quantitative expression analysis was performed in a 40 cycle RT-PCR. After normalization to HPRT (sense 5′-TGA CAC TGG CAA AAC AAT GCA-3′; antisense 5′-GGT CCT TTT CAC CAG CAA GCT-3′, 62° C. annealing) gene-specific transcripts in tumor samples were quantified relative to normal tissues using ΔΔCT calculation.

siRNA Duplexes

The SEQ ID NO:540 siRNA duplexes (Qiagen, Hilden, Germany) were directed against target sequences 5′-NNC CAC AGA AGG UAC CAG UUA-3′ (siRNA#1; sense (5′-CCA CAG AAG GUA CCA GUU AUU-3′), antisense (5′-UAA CUG GUA CCU UCU GUG GUU-3′) and 5′-NNC AGC AAG ACU CCC UCU AAA-3′ (siRNA#2; sense (5′-CAG CAA GAC UCC CUC UAA AUU-3′), antisense (5′-UUU AGA GGG AGU CUU GCU GUU-3′) of the SEQ ID NO:540 mRNA sequence.

### Cell Proliferation Analysis

24 h after transfection with siRNA duplexes 1×10⁴ cells were cultured for 48 h in medium supplemented with 10% FCS. Proliferation was analyzed by measuring the incorporation of BrdU into newly synthesized DNA strands using the DELFIA cell proliferation Kit (Perkin Elmer, Boston, Mass.) according to the manufacturer's instructions on a Wallac Victor² multi-label counter (Perkin Elmer, Boston, Mass.).

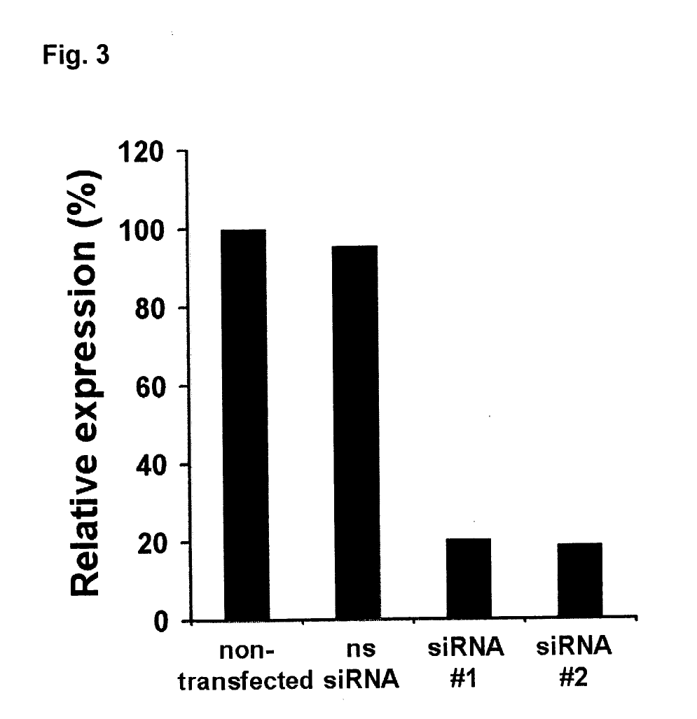

FIG. 3 shows the quantification of SEQ ID NO:540 mRNA expression in MCF-7 breast cancer cells by real-time RT-PCR 24 h after transfection with siRNA oligos. Compared to non-transfected cells and cells transfected with non-silencing (ns) siRNA both SEQ ID NO:540-specific siRNAs (siRNA#1 (SEQ ID NO:630, 631), siRNA#2 (SEQ ID NO:632, 633)) induce robust silencing of SEQ ID NO:540 expression.

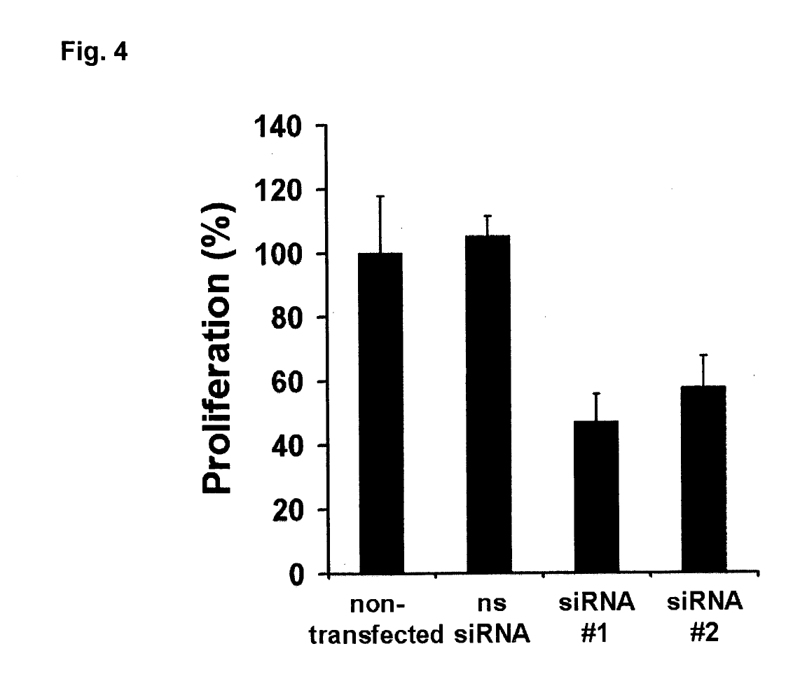

FIG. 4 shows that silencing of SEQ ID NO:540 expression by transfection with siRNA oligos results in impaired proliferation of MCF-7 breast cancer cells. Proliferation was quantified 96 h after transfection with siRNAs by measuring incorporation of BrdU in newly synthesized DNA strands. These results show that SEQ ID NO:540 is a positive factor for the proliferation of breast cancer cells.

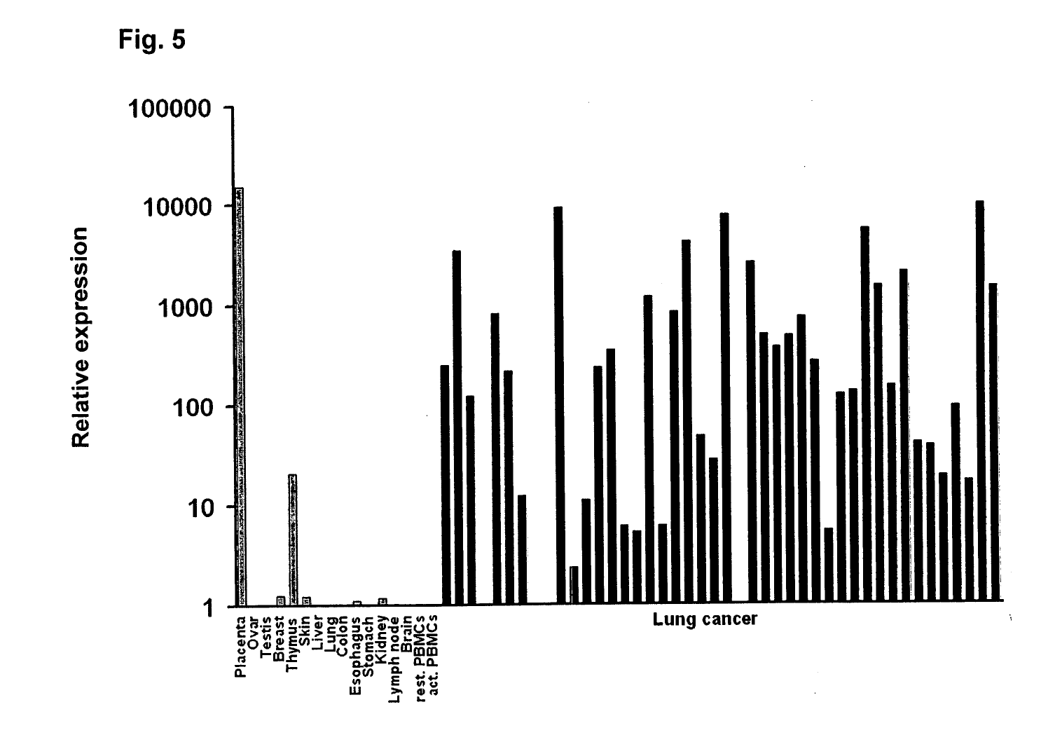

The nucleotide sequence according to SEQ ID NO:541 was deduced from SEQ ID NO:65 and codes for a 177 aa protein (SEQ ID NO:542) of unknown function. Expression of SEQ ID NO:541 in normal and cancerous tissues was quantified by real-time RT-PCR using sequence-specific oligos (SEQ ID NO:543, 544); see FIG. 5. In normal tissues SEQ ID NO:541 is highly expressed in placenta and shows only weak expression in thymus. SEQ ID NO:541 is overexpressed in lung cancer. Based on these expression results, SEQ ID NO:541 and its expression products qualify as molecular markers and/or target candidates for targeted therapies, in particular for this particular tumor type.

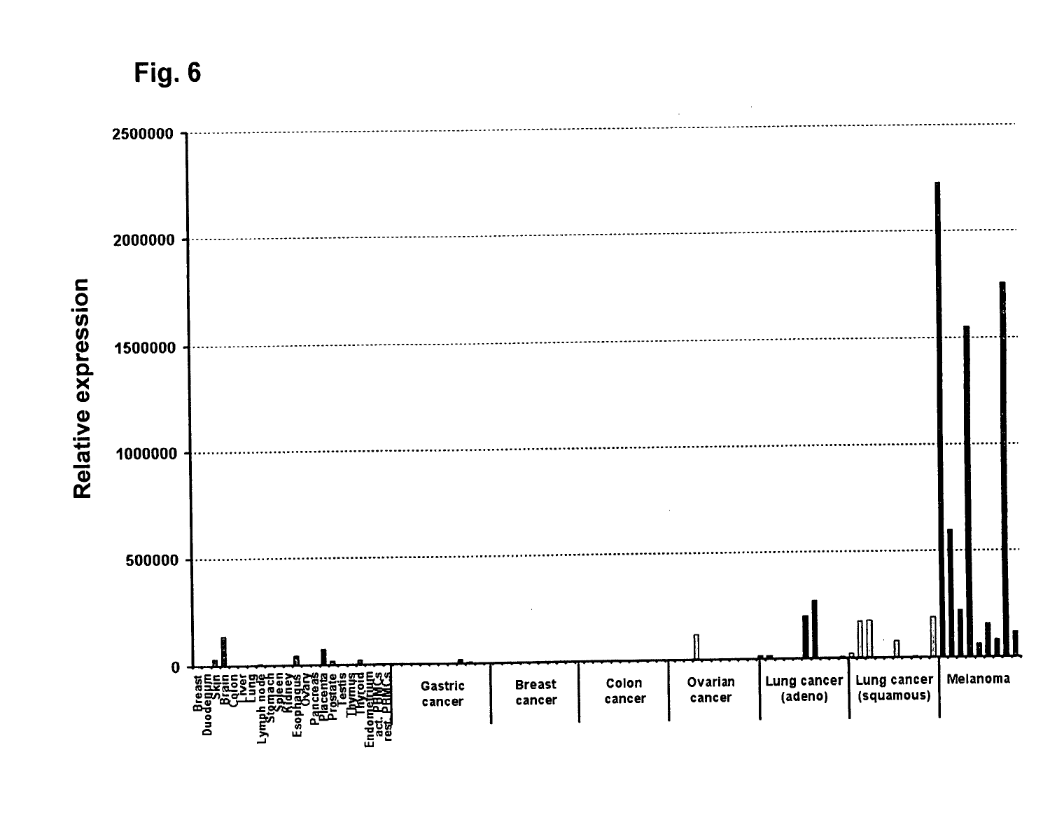

The nucleotide sequence according to SEQ ID NO:545 was deduced from SEQ ID NO:249 and codes for a member of the solute carrier (SLC) group of membrane proteins (SEQ ID NO:546). As is typical of integral membrane proteins, SLCs contain a number of hydrophobic transmembrane alpha helices connected to each other by hydrophilic intra- or extra-cellular loops. Depending on the SLC, these transporters are functional as either monomers or obligate homo- or hetero-oligomers. The protein encoded by SEQ ID NO:545 is a cell surface protein. Expression of SEQ ID NO:545 in normal and cancerous tissues was quantified by real-time RT-PCR using sequence-specific oligos (SEQ ID NO:547, 548); see FIG. 6. Compared to normal tissues, SEQ ID NO:545 is overexpressed in malignant melanomas. Based on these expression results, SEQ ID NO:545 and its expression products qualify as molecular markers and/or target candidates for targeted therapies, in particular for this particular tumor type.

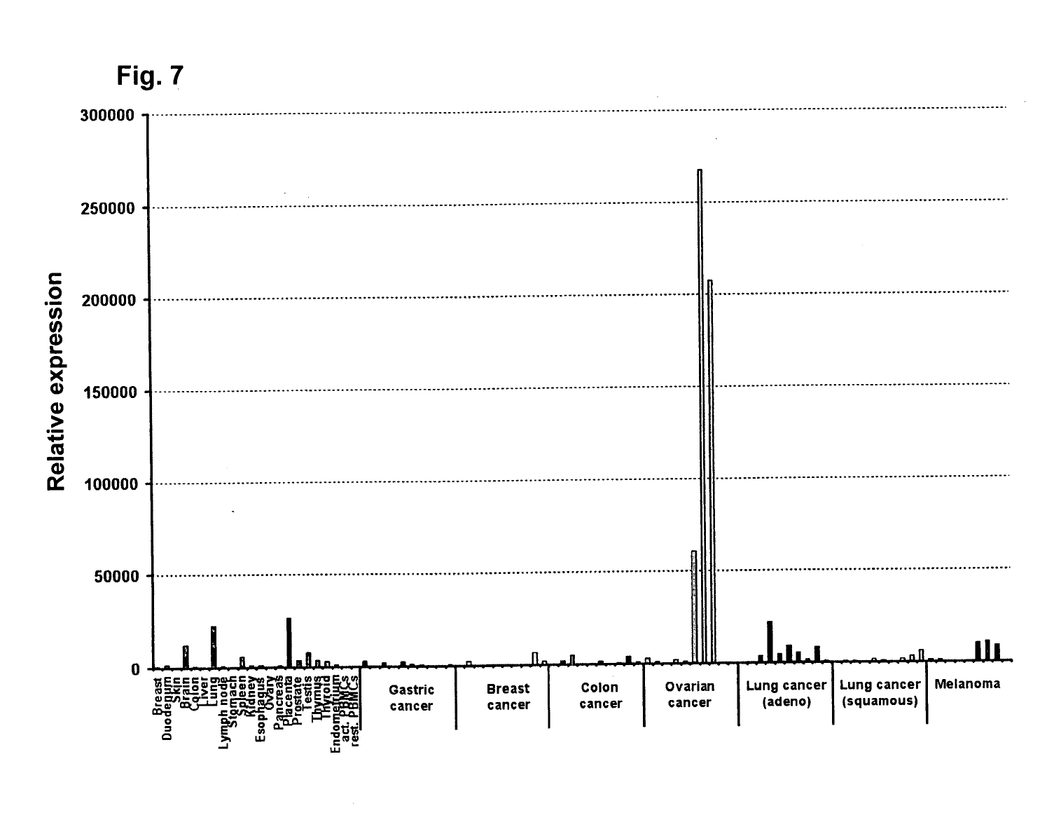

The nucleotide sequence according to SEQ ID NO:549 was deduced from SEQ ID NO:4 and codes for a 763 aa protein (SEQ ID NO:550) of unknown function. The protein harbors two potential transmembrane domains and a typical fibronectin type III domain. Fibronectin is a high-molecular-weight extracellular matrix glycoprotein that binds to membrane spanning receptor proteins (integrins). In addition to integrins, they also bind extracellular matrix components such as collagen, fibrin and heparan sulfate. The protein encoded by SEQ ID NO:549 might represent a hitherto unknown new fibronection-like protein. Expression of SEQ ID NO:549 in normal and cancerous tissues was quantified by real-time RT-PCR using sequence-specific oligos (SEQ ID NO:551, 552); see FIG. 7. Compared to normal tissues, SEQ ID NO:549 is overexpressed in ovarian cancer. Based on these expression results, SEQ ID NO:549 and its expression products qualify as molecular markers and/or target candidates for targeted therapies, in particular of this particular tumor type.

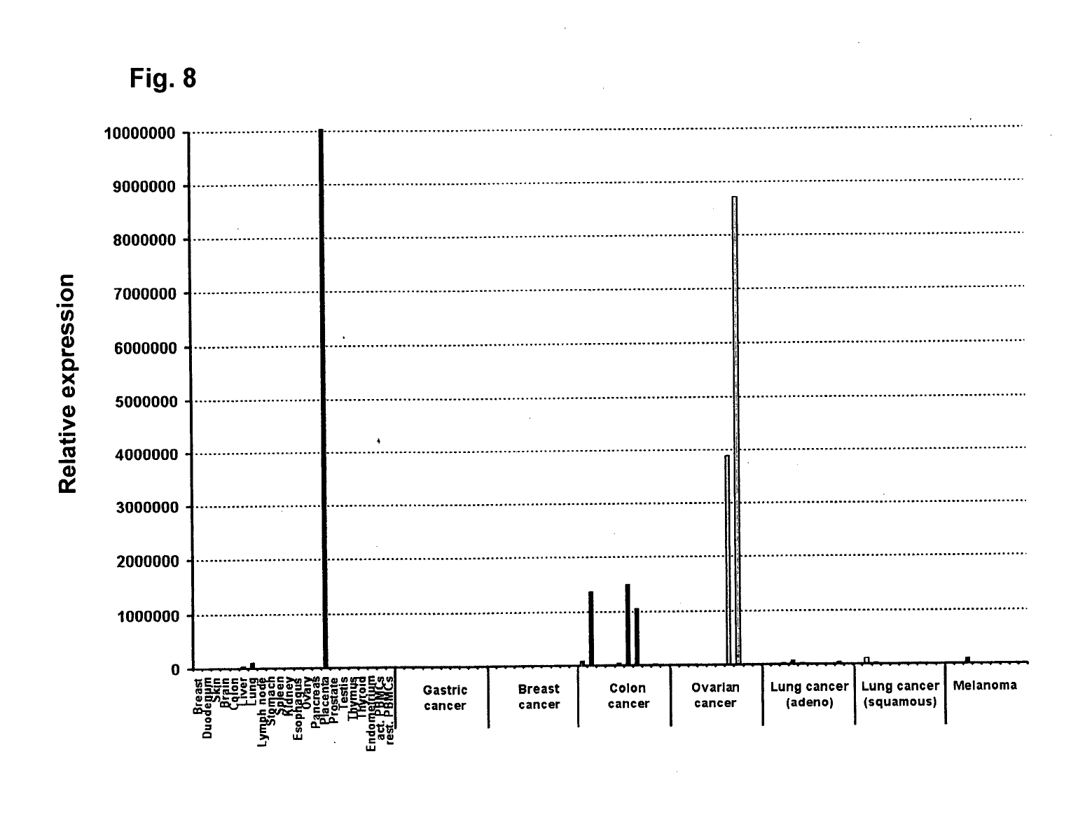

The nucleotide sequence according to SEQ ID NO:553 was deduced from SEQ ID NO:156 and codes for a 496 aa protein (SEQ ID NO:554) of unknown function. The protein harbors a potential transmembrane protein. Expression of SEQ ID NO:553 in normal and cancerous tissues was quantified by real-time RT-PCR using sequence-specific oligos (SEQ ID NO:555, 556); see FIG. 8. In normal tissues SEQ ID NO:553 is highly expressed in placenta. Compared to other normal tissues, SEQ ID NO:553 is overexpressed in colon cancer and ovarian cancer. Based on these expression results, SEQ ID NO:553 and its expression products qualify as molecular markers and/or target candidates for targeted therapies, in particular for these particular tumor types.

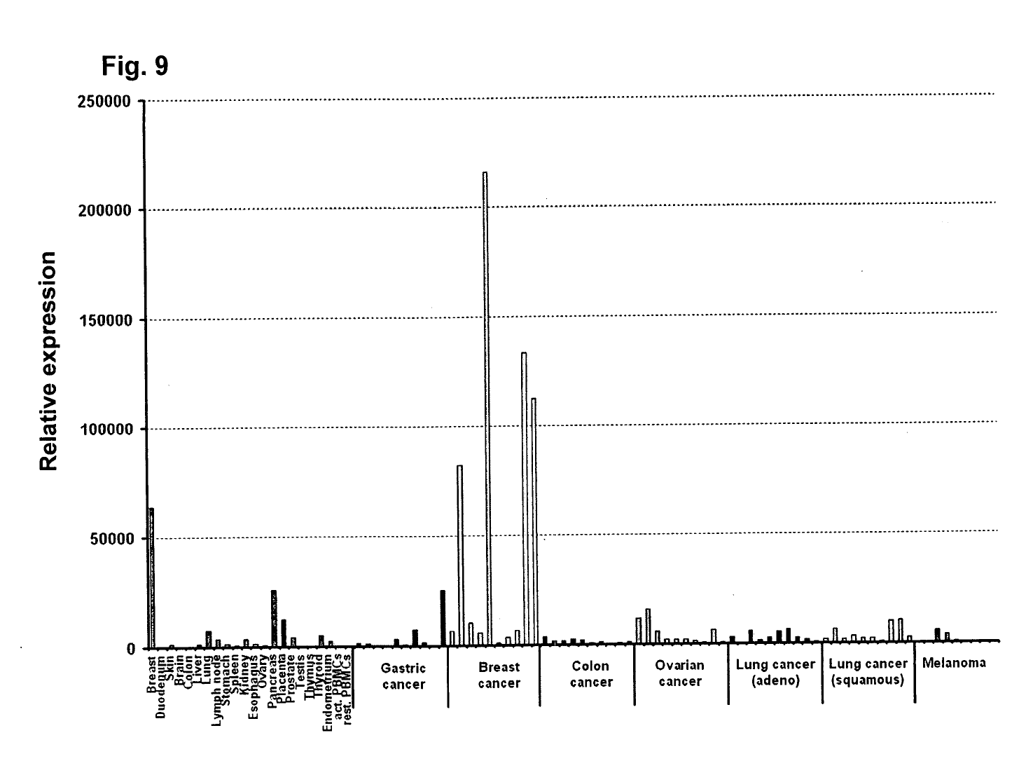

The nucleotide sequence according to SEQ ID NO:557 was deduced from SEQ ID NO:273. SEQ ID NO:557 represents a partial cDNA with no apparent open reading frame. Expression of SEQ ID NO:557 in normal and cancerous tissues was quantified by real-time RT-PCR using sequence-specific oligos (SEQ ID NO:558, 559); see FIG. 9. In normal tissues high expression of SEQ ID NO:557 is detectable in breast. Compared to normal tissues, SEQ ID NO:557 is overexpressed in breast cancer. Based on these expression results, SEQ ID NO:557 and its expression products qualify as molecular markers and/or target candidates for targeted therapies, in particular for this particular tumor type.

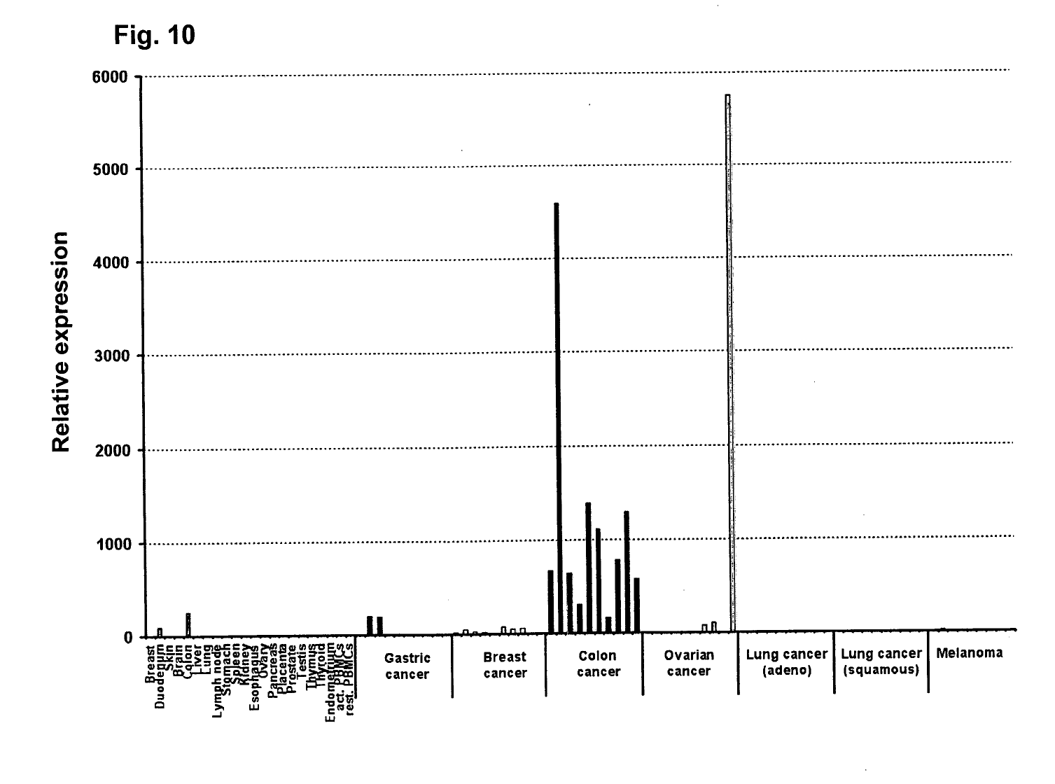

The nucleotide sequence according to SEQ ID NO:560 was deduced from SEQ ID NO:135. SEQ ID NO:560 has no apparent open reading frame. Expression of SEQ ID NO:560 in normal and cancerous tissues was quantified by real-time RT-PCR using sequence-specific oligos (SEQ ID NO:561, 562); see FIG. 10. In normal tissues expression of SEQ ID NO:560 is detectable in duodenum and colon. Compared to normal tissues, SEQ ID NO:560 is overexpressed in colon cancer and ovarian cancer. Based on these expression results, SEQ ID NO:560 and its expression products qualify as molecular markers and/or target candidates for targeted therapies, in particular for these particular tumor types.

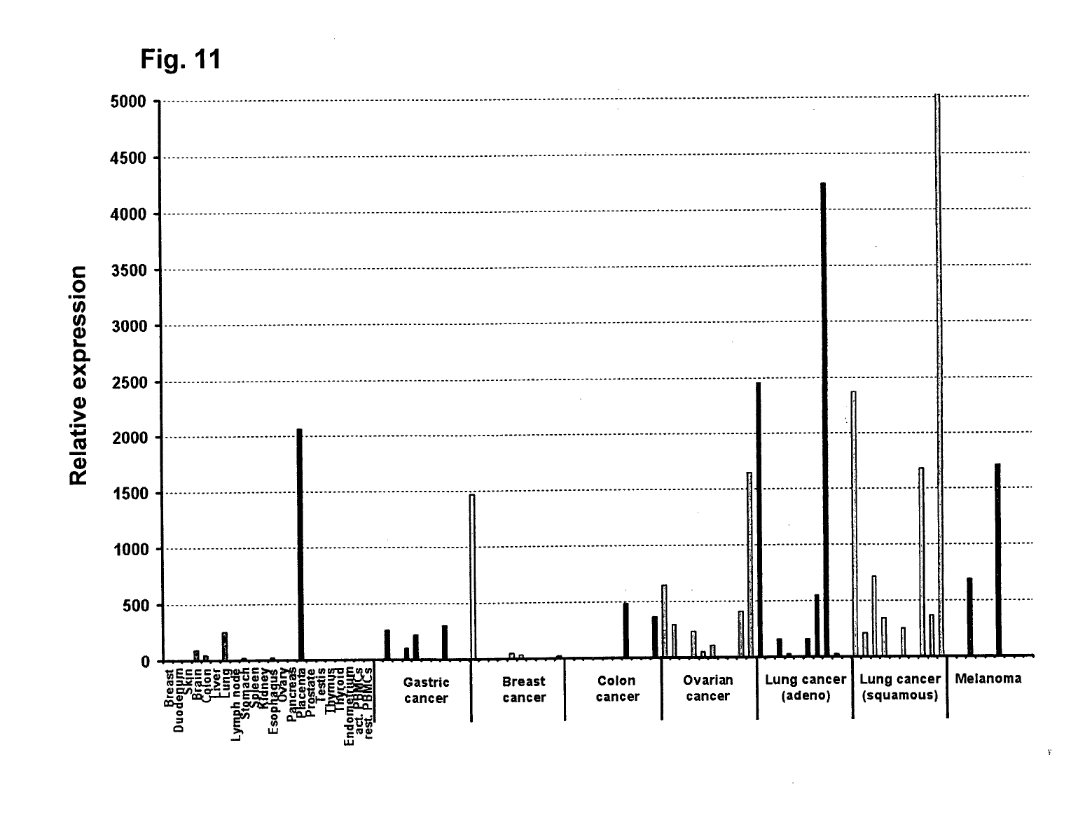

The nucleotide sequence according to SEQ ID NO:563 was deduced from SEQ ID NO:177. SEQ ID NO:563 has no apparent open reading frame. Expression of SEQ ID NO:563 in normal and cancerous tissues was quantified by real-time RT-PCR using sequence-specific oligos (SEQ ID NO:564, 565); see FIG. 11. SEQ ID NO:563 is highly expressed in placenta. Compared to normal tissues, SEQ ID NO:563 is overexpressed in breast cancer, colon cancer, ovarian cancer, lung cancer and melanoma. Based on these expression results, SEQ ID NO:563 and its expression products qualify as molecular markers and/or target candidates for targeted therapies, in particular for these particular tumor types.

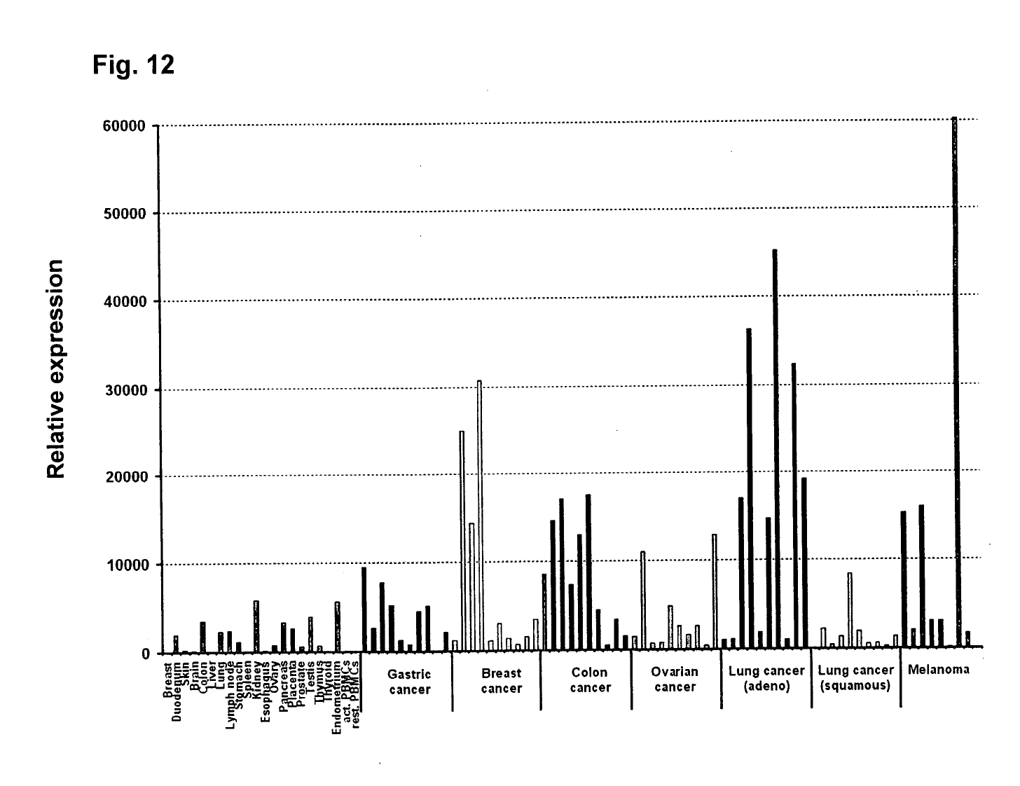

The nucleotide sequence according to SEQ ID NO:566 was deduced from SEQ ID NO:149 and codes for a 155 aa protein (SEQ ID NO:567) of unknown function. The protein sequence is partially homologous to members of the tumor necrosis factor receptor superfamily and harbors a potential transmembrane domain. The protein encoded by SEQ ID NO:566 might represent a new member of the tumor necrosis factor receptor superfamily. Expression of SEQ ID NO:566 in normal and cancerous tissues was quantified by real-time RT-PCR using sequence-specific oligos (SEQ ID NO:568, 569); see FIG. 12. Compared to normal tissues, SEQ ID NO:566 is overexpressed in gastric cancer, breast cancer, colon cancer, ovarian cancer, lung cancer and melanoma. Based on these expression results, SEQ ID NO:566 and its expression products qualify as molecular markers and/or target candidates for targeted therapies, in particular for these particular tumor types.

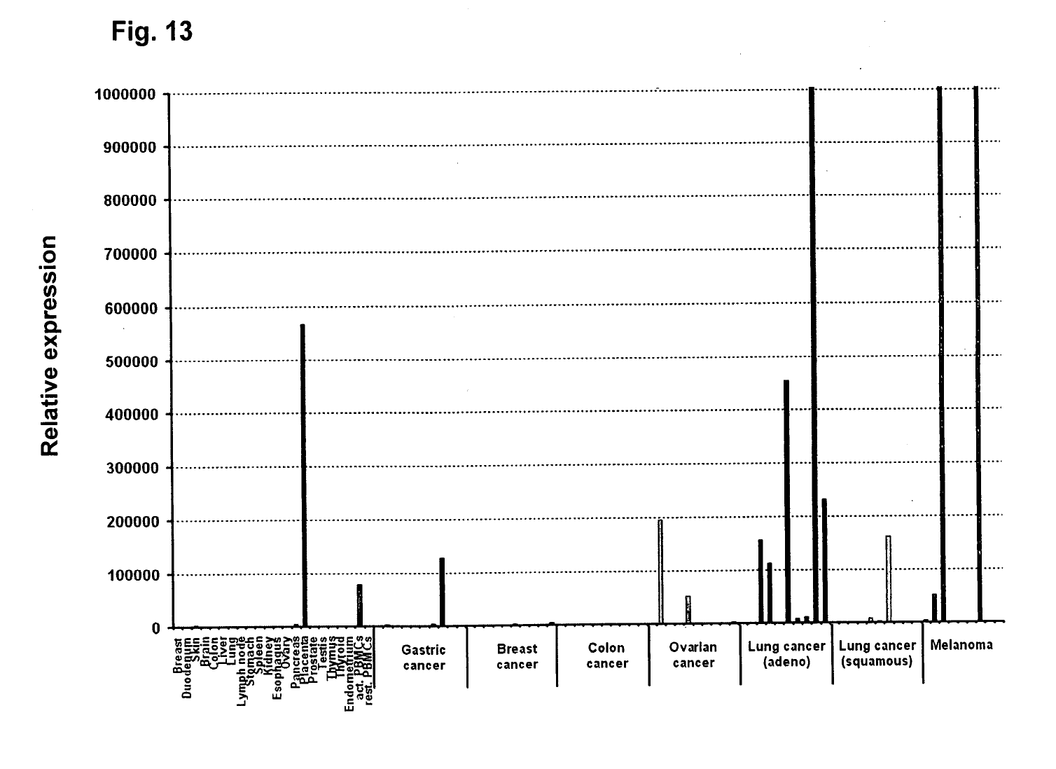

The nucleotide sequence according to SEQ ID NO:570 was deduced from SEQ ID NO:53 and codes for a member of the kernel lipocain superfamily (SEQ ID NO:571). These secreted glycoproteins have distinct and essential roles in regulating an uterine environment suitable for pregnancy and in the timing and occurrence of the appropriate sequence of events in the fertilization process. Expression of SEQ ID NO:570 in normal and cancerous tissues was quantified by real-time RT-PCR using sequence-specific oligos (SEQ ID NO:572, 573); see FIG. 13. SEQ ID NO:570 is highly expressed in placenta. Compared to other normal tissues, SEQ ID NO:570 is overexpressed in ovarian cancer, lung cancer and melanoma. Based on these expression results, SEQ ID NO:570 and its expression products qualify as molecular markers and/or target candidates for targeted therapies, in particular for these particular tumor types.

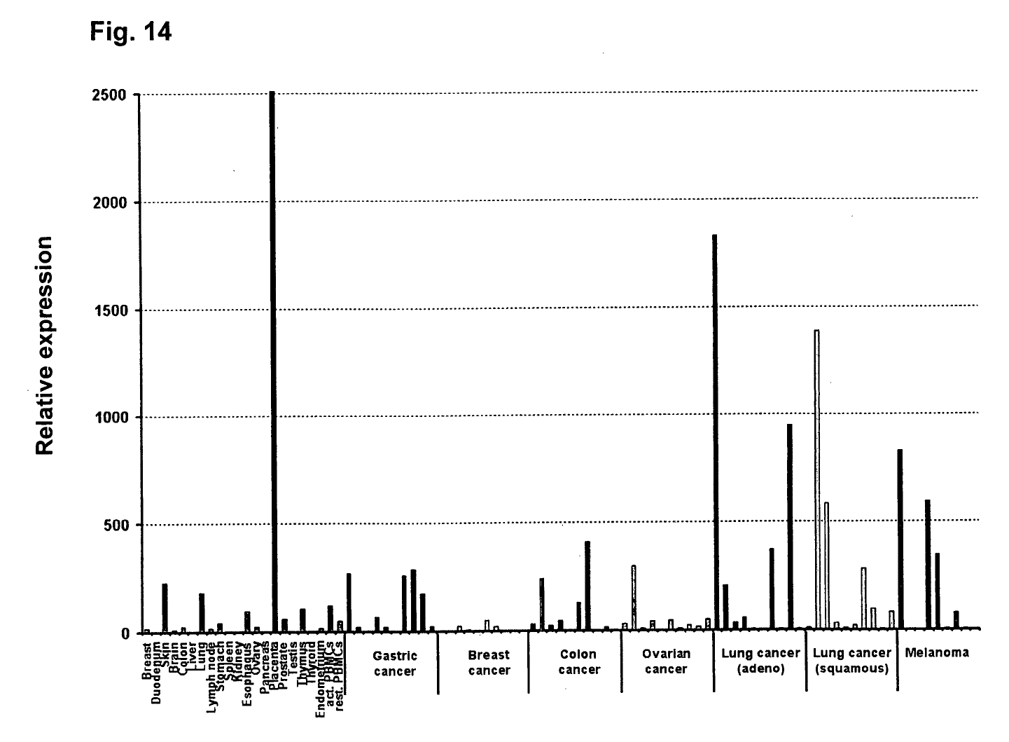

The nucleotide sequence according to SEQ ID NO:574 has no apparent open reading frame. Expression of SEQ ID NO:574 in normal and cancerous tissues was quantified by real-time RT-PCR using sequence-specific oligos (SEQ ID NO:575, 576); see FIG. 14. SEQ ID NO:574 is highly expressed in placenta. Compared to other normal tissues, SEQ ID NO:574 is overexpressed in lung cancer and melanoma. Based on these expression results, SEQ ID NO:574 and its expression products qualify as molecular markers and/or target candidates for targeted therapies, in particular for these particular tumor types.

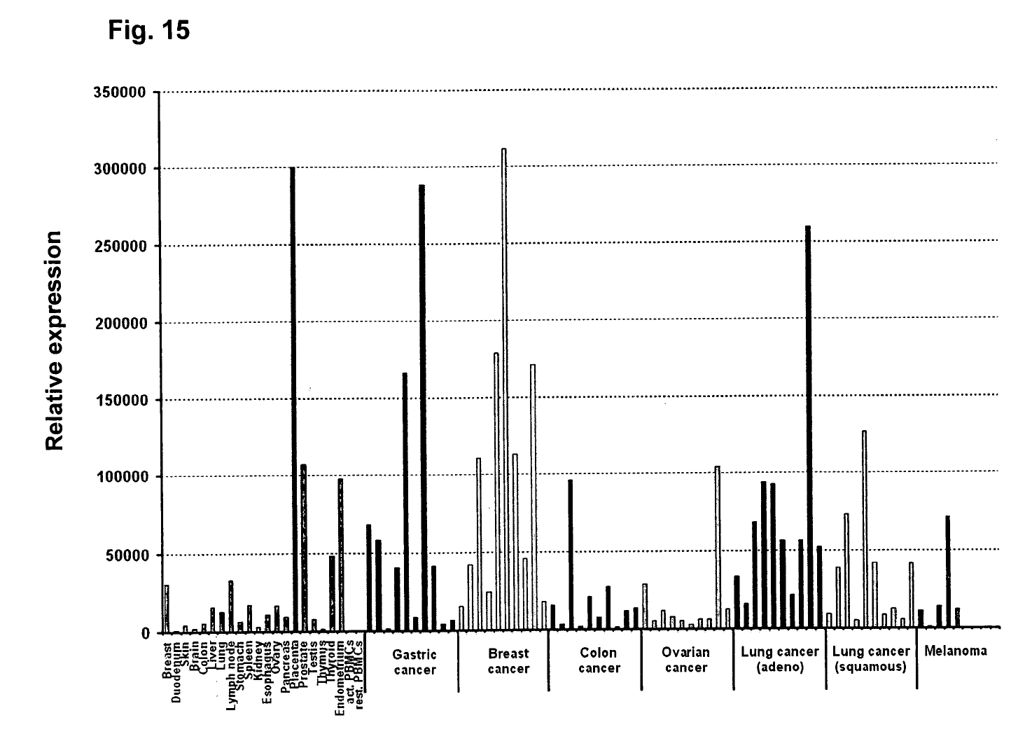

The nucleotide sequence according to SEQ ID NO:577 was deduced from SEQ ID NO:20. SEQ ID NO:577 represents a partial cDNA with no apparent open reading frame. Expression of SEQ ID NO:577 in normal and cancerous tissues was quantified by real-time RT-PCR using sequence-specific oligos (SEQ ID NO:578, 579); see FIG. 15. SEQ ID NO:577 is highly expressed in placenta. Compared to other normal tissues, SEQ ID NO:577 is overexpressed in gastric cancer, breast cancer and lung cancer. Based on these expression results, SEQ ID NO:577 and its expression products qualify as molecular markers and/or target candidates for targeted therapies, in particular for these particular tumor types.

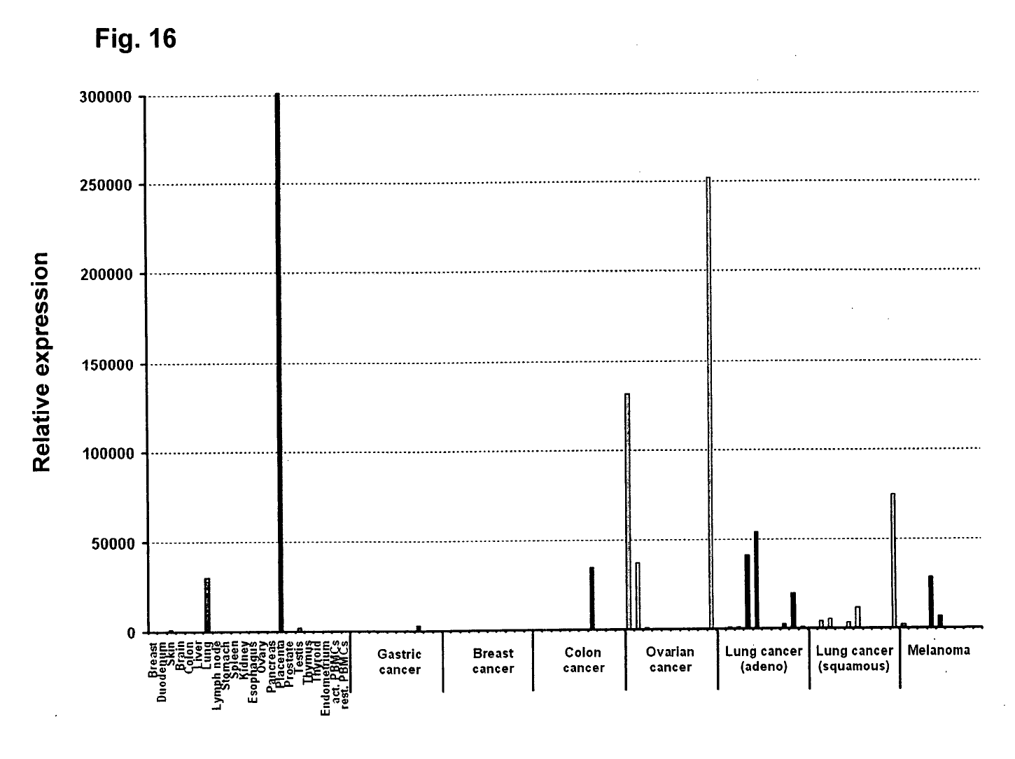

The nucleotide sequence according to SEQ ID NO:580 was deduced from SEQ ID NO:32. SEQ ID NO:580 represents a partial cDNA with no apparent open reading frame. Expression of SEQ ID NO:580 in normal and cancerous tissues was quantified by real-time RT-PCR using sequence-specific oligos (SEQ ID NO:581, 582); see FIG. 16. SEQ ID NO:580 is highly expressed in placenta. Compared to other normal tissues, SEQ ID NO:580 is overexpressed in ovarian cancer and lung cancer. Based on these expression results, SEQ ID NO:580 and its expression products qualify as molecular markers and/or target candidates for targeted therapies, in particular for these particular tumor types.

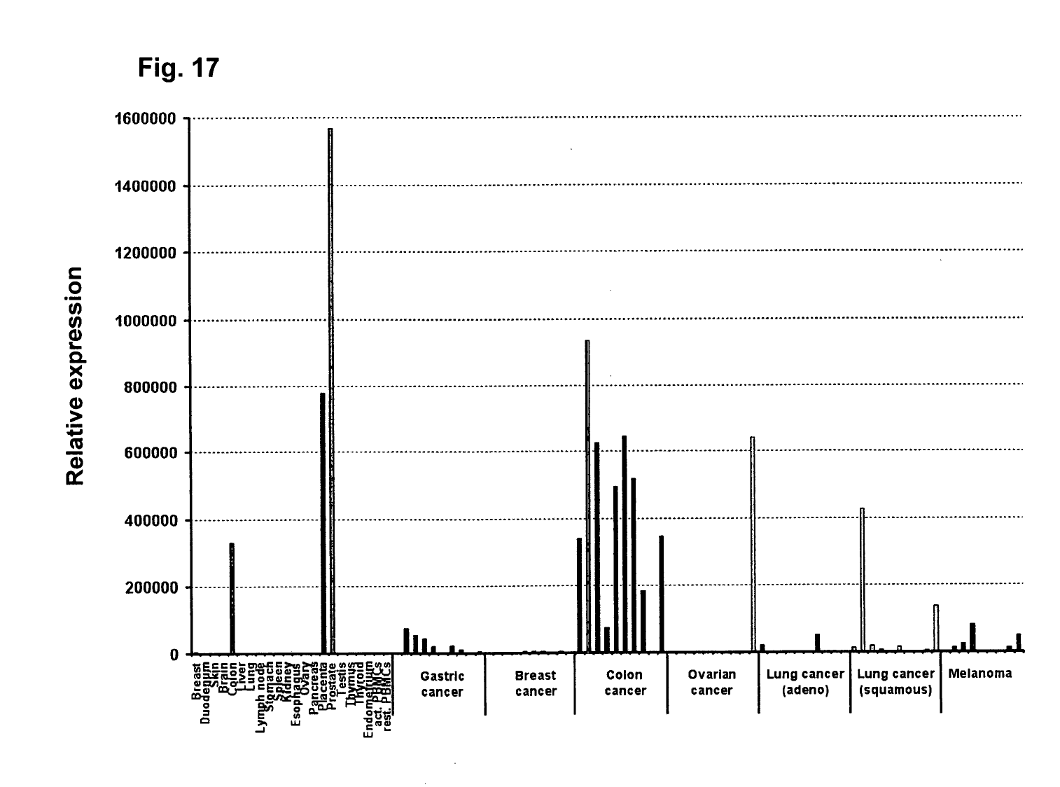

The nucleotide sequence according to SEQ ID NO:583 was deduced from SEQ ID NO:257 and codes for a member of the homeobox class of transcription factors (SEQ ID NO:584). Expression of these proteins is spatially and temporally regulated during embryonic development. Expression of SEQ ID NO:583 in normal and cancerous tissues was quantified by real-time RT-PCR using sequence-specific oligos (SEQ ID NO:585, 586); see FIG. 17. SEQ ID NO:583 is highly expressed in placenta and prostate. Compared to other normal tissues, SEQ ID NO:583is overexpressed in colon cancer, ovarian cancer and lung cancer. Based on these expression results, SEQ ID NO:583 and its expression products qualify as molecular markers and/or target candidates for targeted therapies, in particular for these particular tumor types.

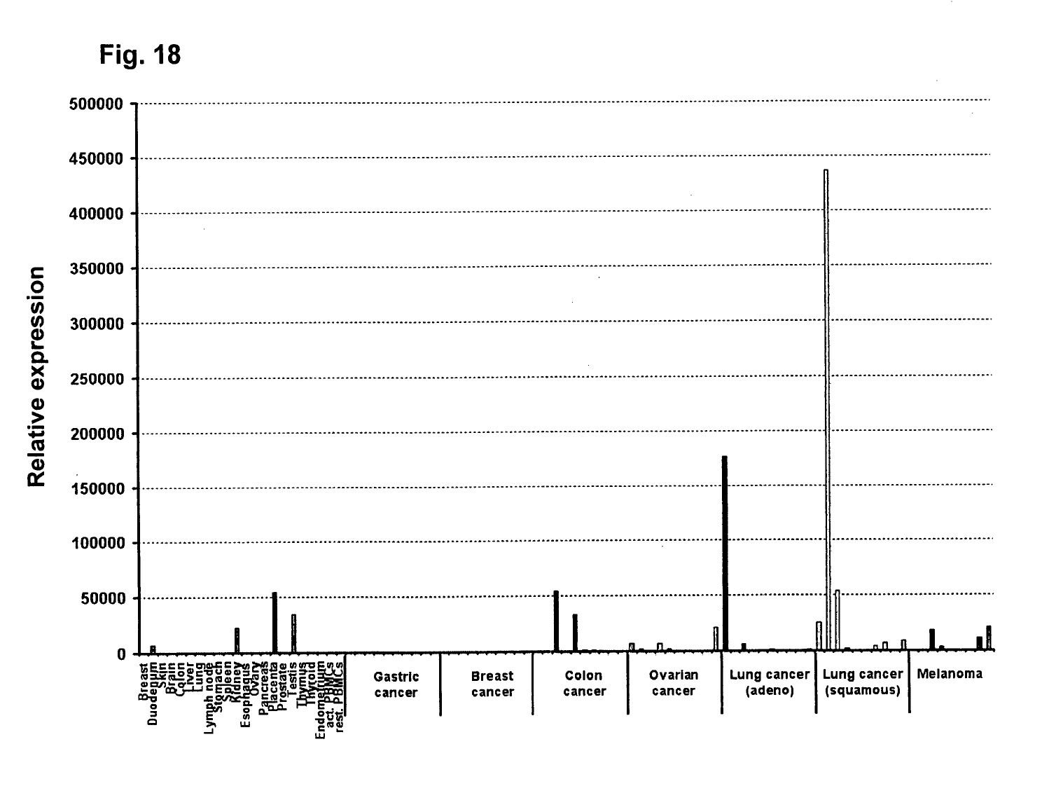

The nucleotide sequence according to SEQ ID NO:587 was deduced from SEQ ID NO:148 and codes for a member of the IGF-II mRNA-binding protein (IMP) family (SEQ ID NO:588). It functions by binding to the 5′ UTR of the insulin-like growth factor 2 (IGF2) mRNA and regulating IGF2 translation. Expression of SEQ ID NO:587 in normal and cancerous tissues was quantified by real-time RT-PCR using sequence-specific oligos (SEQ ID NO:589, 590); see FIG. 18. Compared to normal tissues, SEQ ID NO:587 is overexpressed in lung cancer. Based on these expression results, SEQ ID NO:587 and its expression products qualify as molecular markers and/or target candidates for targeted therapies, in particular for this particular tumor type.

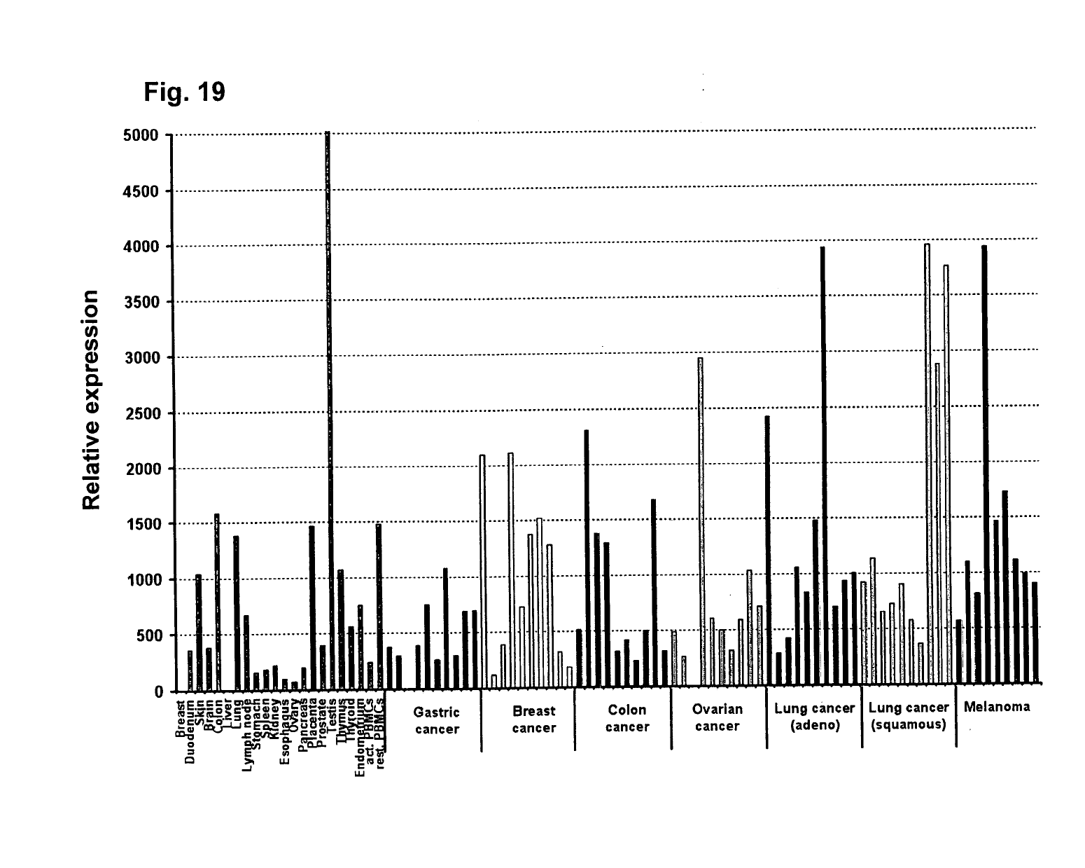

The nucleotide sequence according to SEQ ID NO:591 was deduced from SEQ ID NO:194 and codes for a 372 aa protein (SEQ ID NO:592) of unknown function. Expression of SEQ ID NO:591 in normal and cancerous tissues was quantified by real-time RT-PCR using sequence-specific oligos (SEQ ID NO:593, 594); see FIG. 19. SEQ ID NO:591 is highly expressed in testis. Compared to other normal tissues, SEQ ID NO:591 is overexpressed in breast cancer, colon cancer, ovarian cancer, lung cancer and melanoma. Based on these expression results, SEQ ID NO:591 and its expression products qualify as molecular markers and/or target candidates for targeted therapies, in particular for these particular tumor types.

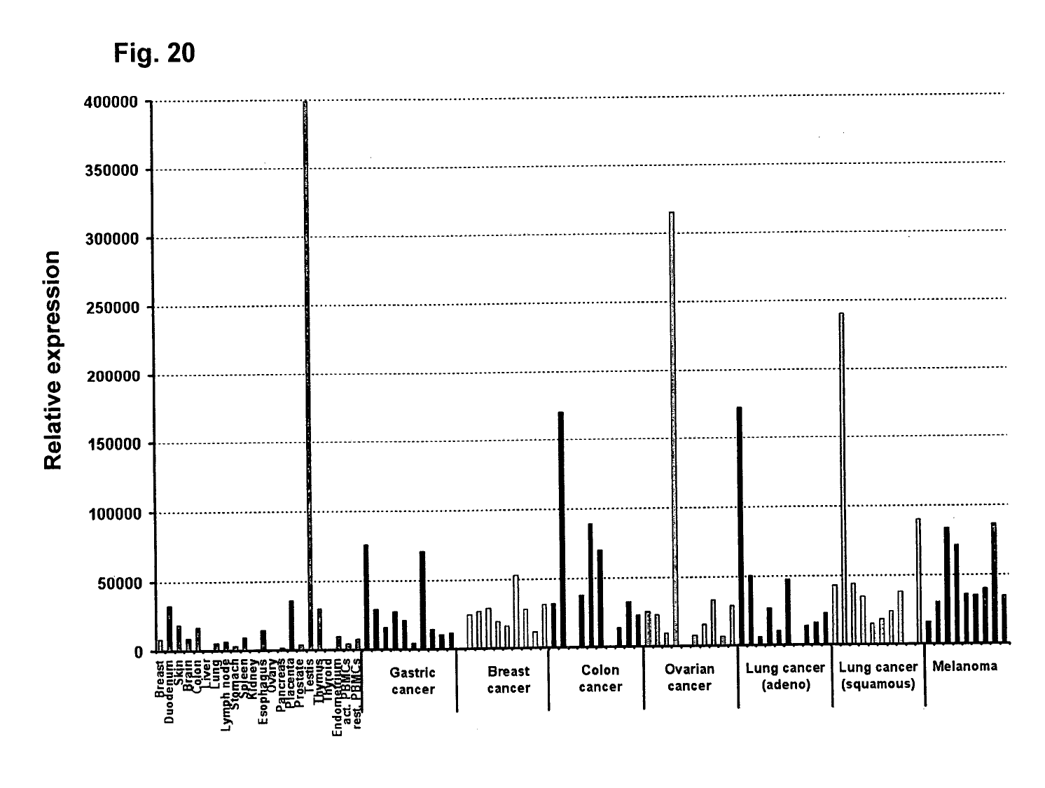

The nucleotide sequence according to SEQ ID NO:595 was deduced from SEQ ID NO:191 and codes for a 357 aa protein (SEQ ID NO:596) of unknown function. Expression of SEQ ID NO:595 in normal and cancerous tissues was quantified by real-time RT-PCR using sequence-specific oligos (SEQ ID NO:597, 598); see FIG. 20. SEQ ID NO:595 is highly expressed in testis. Compared to other normal tissues, SEQ ID NO:595 is overexpressed in gastric cancer, colon cancer, ovarian cancer, lung cancer and melanoma. Based on these expression results, SEQ ID NO:595 and its expression products qualify as molecular markers and/or target candidates for targeted therapies, in particular for these particular tumor types.

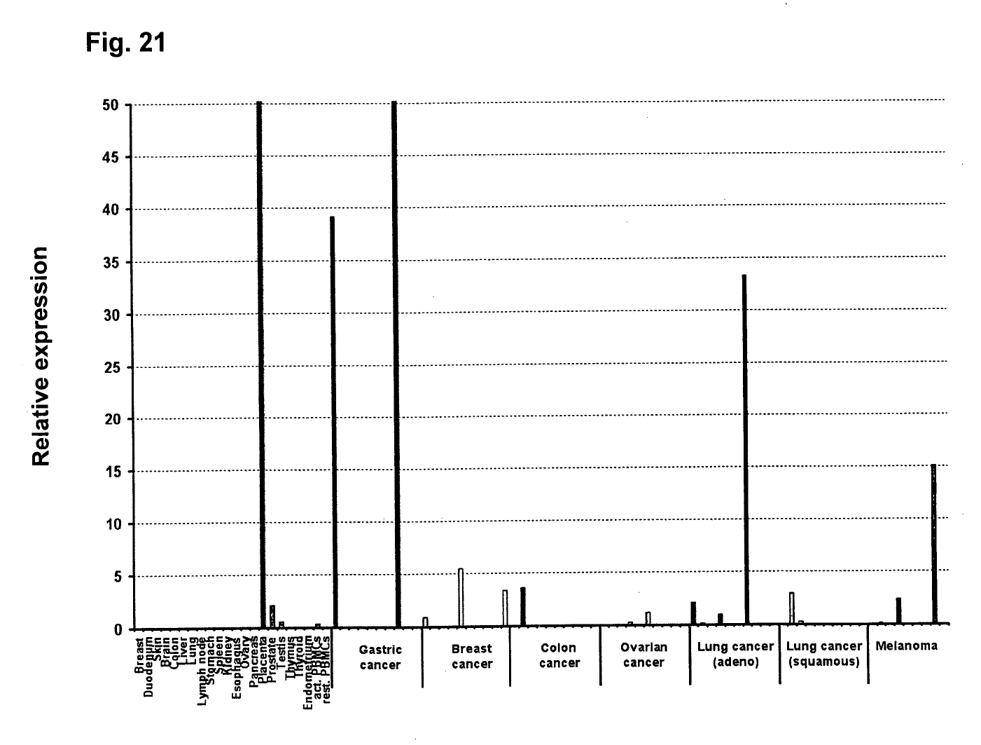

The nucleotide sequence according to SEQ ID NO:599 was deduced from SEQ ID NO:18 and has no apparent open reading frame. Expression of SEQ ID NO:599 in normal and cancerous tissues was quantified by real-time RT-PCR using sequence-specific oligos (SEQ ID NO:600, 601); see FIG. 21. SEQ ID NO:599 is highly expressed in placenta. Compared to other normal tissues, SEQ ID NO:599 is overexpressed in gastric cancer, breast cancer, lung cancer and melanoma. Based on these expression results, SEQ ID NO:599 and its expression products qualify as molecular markers and/or target candidates for targeted therapies, in particular for these particular tumor types.

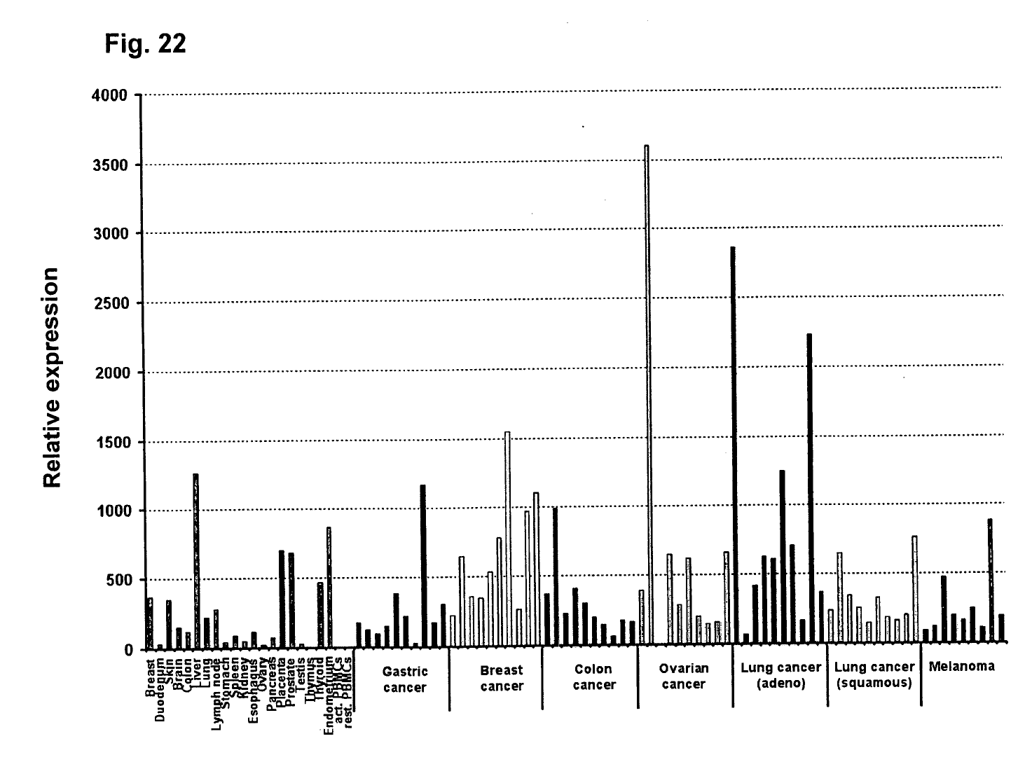

The nucleotide sequence according to SEQ ID NO:602 was deduced from SEQ ID NO:133 and codes for a member of the von Willebrand factor domain superfamily of extracellular matrix proteins (SEQ ID NO:603). Expression of SEQ ID NO:602 in normal and cancerous tissues was quantified by real-time RT-PCR using sequence-specific oligos (SEQ ID NO:604, 605); see FIG. 22. Compared to normal tissues, SEQ ID NO:602 is overexpressed in ovarian cancer and lung cancer. Based on these expression results, SEQ ID NO:602 and its expression products qualify as molecular markers and/or target candidates for targeted therapies, in particular for these particular tumor types.

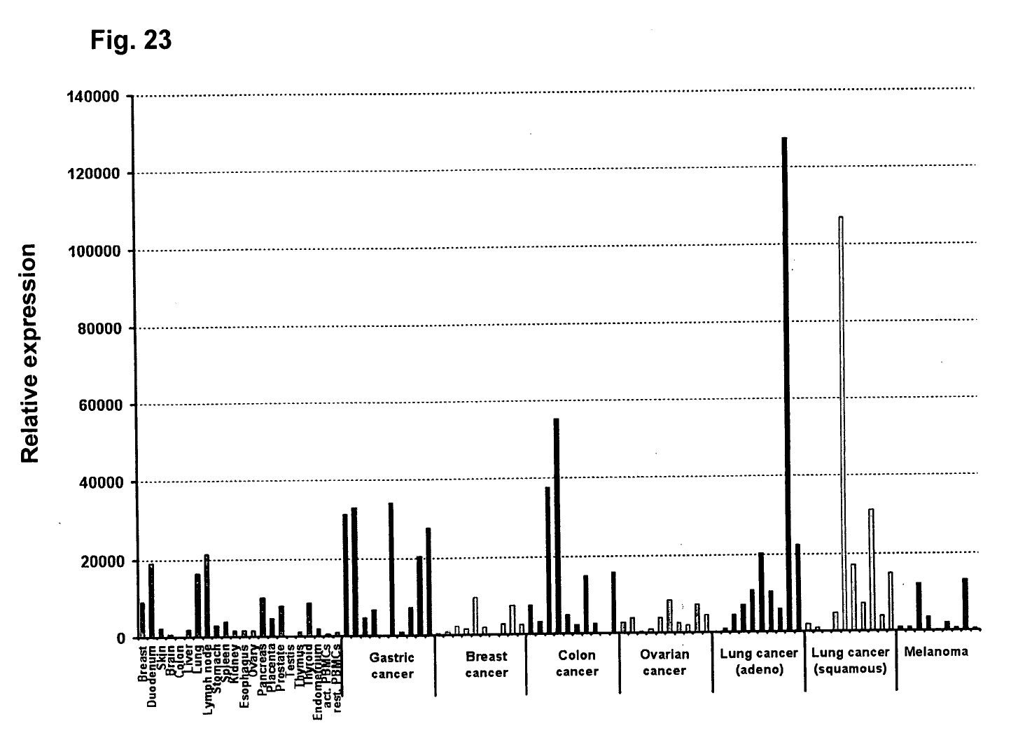

The nucleotide sequence according to SEQ ID NO:606 was deduced from SEQ ID NO:128 and codes for a member of the Borg family of CDC42 effector proteins (SEQ ID NO:607). Borg family proteins contain a CRIB (Cdc42/Rac interactive-binding) domain. They bind to, and negatively regulate the function of CDC42. CDC42, a small Rho GTPase, regulates the formation of F-actin-containing structures through its interaction with the downstream effector proteins. Expression of SEQ ID NO:606 in normal and cancerous tissues was quantified by real-time RT-PCR using sequence-specific oligos (SEQ ID NO:608, 609); see FIG. 23. Compared to normal tissues, SEQ ID NO:606 is overexpressed in gastric cancer, colon cancer and lung cancer. Based on these expression results, SEQ ID NO:606 and its expression products qualify as molecular markers and/or target candidates for targeted therapies, in particular for these particular tumor types.

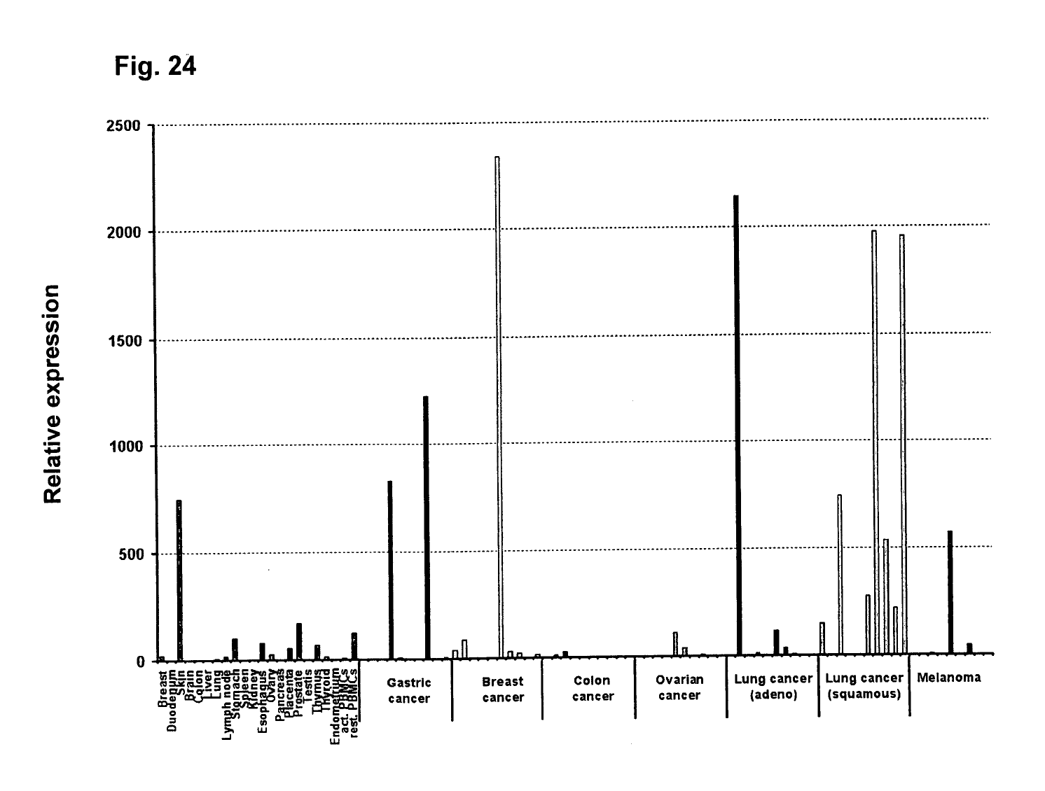

The nucleotide sequence according to SEQ ID NO:610 was deduced from SEQ ID NO:118 and has no apparent open reading frame. Expression of SEQ ID NO:610 in normal and cancerous tissues was quantified by real-time RT-PCR using sequence-specific oligos (SEQ ID NO:611, 612); see FIG. 24. Compared to normal tissues, SEQ ID NO:610 is overexpressed in gastric cancer, breast cancer and lung cancer. Based on these expression results, SEQ ID NO:610 and its expression products qualify as molecular markers and/or target candidates for targeted therapies, in particular for these particular tumor types.

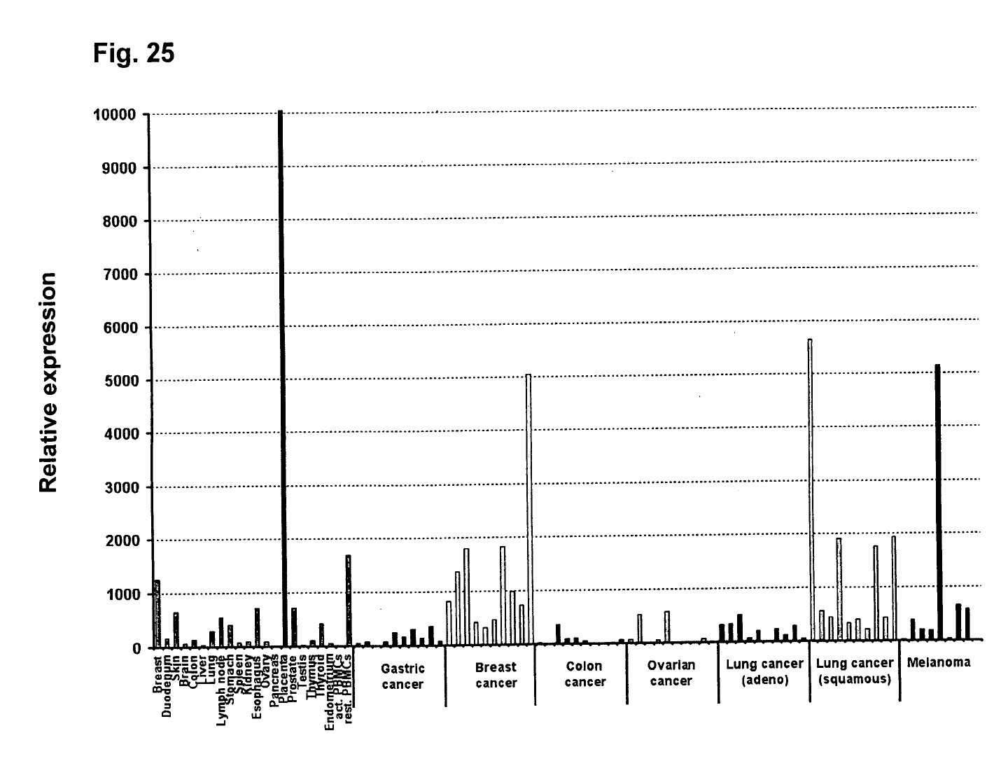

The nucleotide sequence according to SEQ ID NO:613 was deduced from SEQ ID NO:116 and codes for a 76 aa protein (SEQ ID NO:614) of unknown function. Expression of SEQ ID NO:613 in normal and cancerous tissues was quantified by real-time RT-PCR using sequence-specific oligos (SEQ ID NO:615, 616); see FIG. 25. SEQ ID NO:613 is highly expressed in placenta. Compared to other normal tissues, SEQ ID NO:613 is overexpressed in breast cancer, lung cancer and melanoma. Based on these expression results, SEQ ID NO:613 and its expression products qualify as molecular markers and/or target candidates for targeted therapies, in particular for these particular tumor types.

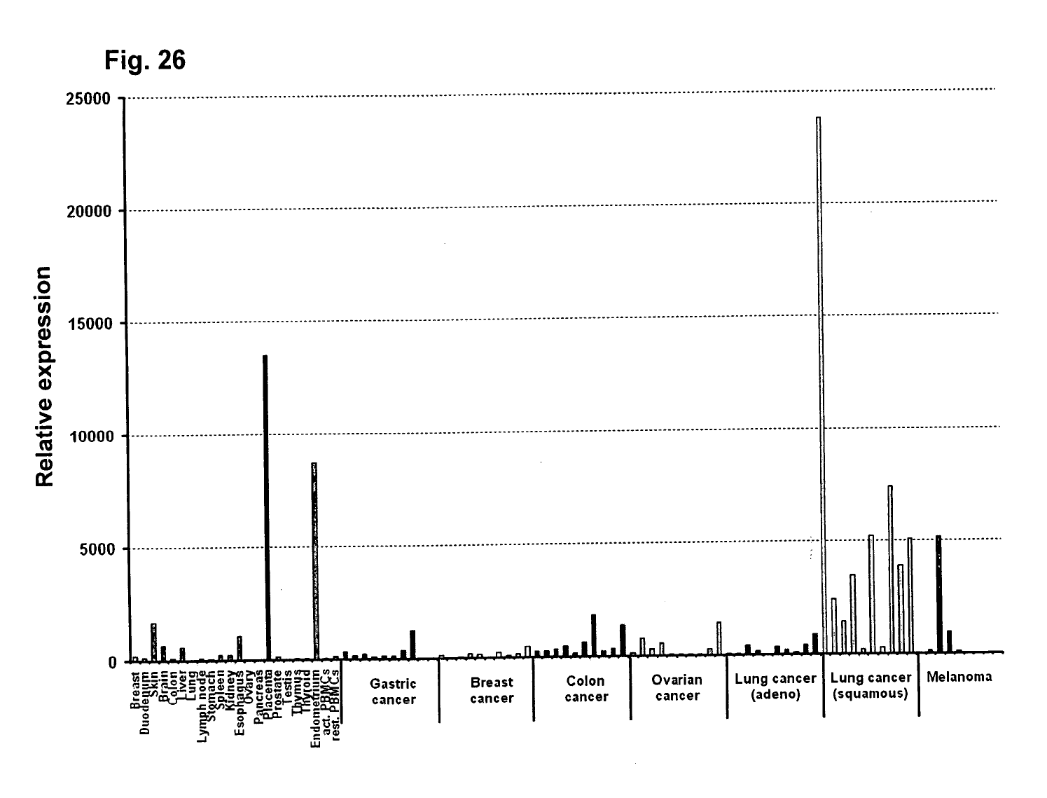

The nucleotide sequence according to SEQ ID NO:617 was deduced from SEQ ID NO:267. SEQ ID NO:617 represents a partial cDNA with no apparent open reading frame. Expression of SEQ ID NO:617 in normal and cancerous tissues was quantified by real-time RT-PCR using sequence-specific oligos (SEQ ID NO:618, 619); see FIG. 26. SEQ ID NO:617 is highy expressed in placenta and endometrium. Compared to other normal tissues, SEQ ID NO:617 is overexpressed in lung cancer and melanoma. Based on these expression results, SEQ ID NO:617 and its expression products qualify as molecular markers and/or target candidates for targeted therapies, in particular for these particular tumor types.

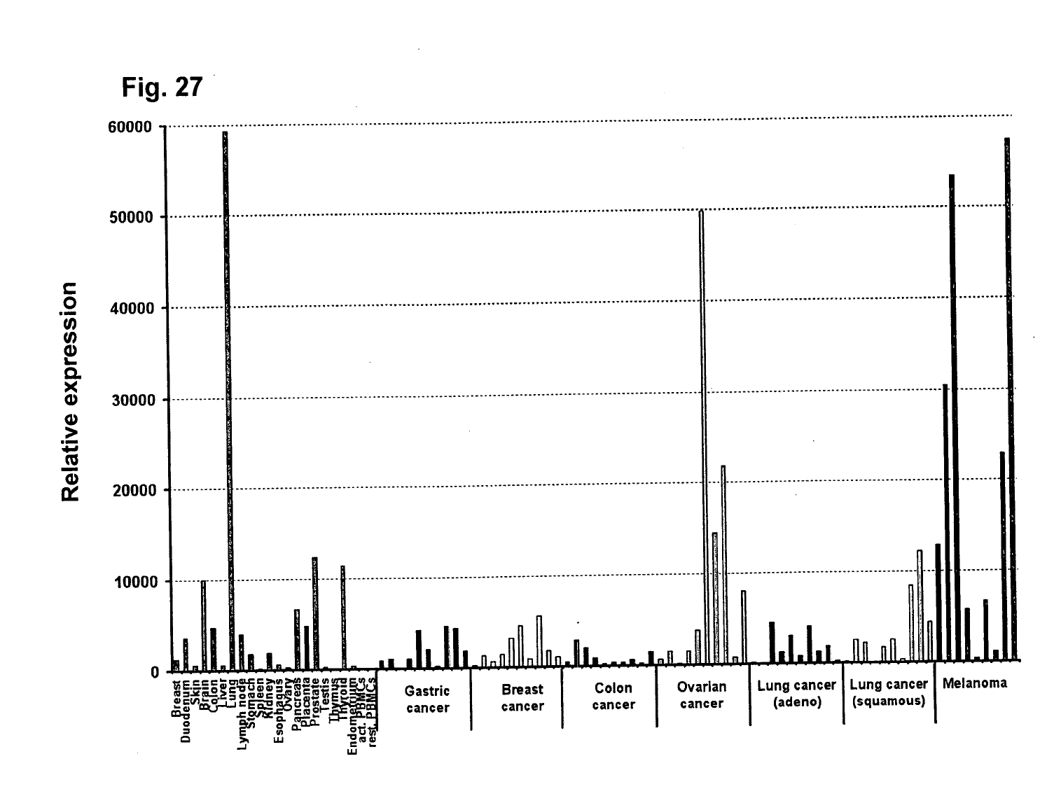

The nucleotide sequence according to SEQ ID NO:620 was deduced from SEQ ID NO:182 and codes for a 829 aa protein (SEQ ID NO:621) harboring multiple putative transmembrane domains and a patched family domain. The transmembrane protein Patched is a receptor for the morphogene Sonic Hedgehog. This protein associates with the smoothened protein to transduce hedgehog signals. SEQ ID NO:620 might represent a novel member of the Patched family. Expression of SEQ ID NO:620 in normal and cancerous tissues was quantified by real-time RT-PCR using sequence-specific oligos (SEQ ID NO:622, 623); see FIG. 27. SEQ ID NO:620 is highly expressed in lung. Compared to other normal tissues, SEQ ID NO:620 is overexpressed in ovarian cancer and melanoma. Based on these expression results, SEQ ID NO:620 and its expression products qualify as molecular markers and/or target candidates for targeted therapies, in particular for these particular tumor types.

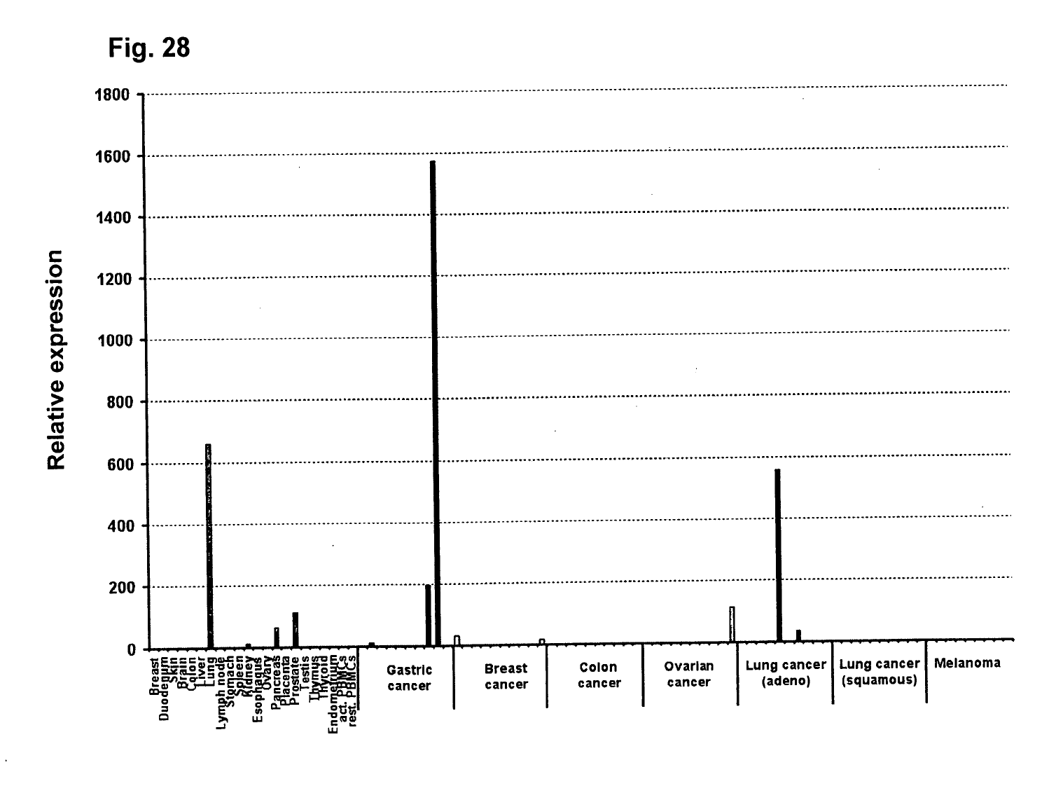

The nucleotide sequence according to SEQ ID NO:624 was deduced from SEQ ID NO:184 and codes for a 323 aa protein (SEQ ID NO:625) similar to TWIK-related acid-sensitive K⁺ channel, a member of the superfamily of potassium channel proteins that contain two pore-forming P domains. Expression of SEQ ID NO:624 in normal and cancerous tissues was quantified by real-time RT-PCR using sequence-specific oligos (SEQ ID NO:626, 627); see FIG. 28. SEQ ID NO:624 is highly expressed in lung. Compared to other normal tissues, SEQ ID NO:624 is overexpressed in gastric cancer and lung cancer. Based on these expression results, SEQ ID NO:624 and its expression products qualify as molecular markers and/or target candidates for targeted therapies, in particular for these particular tumor types.

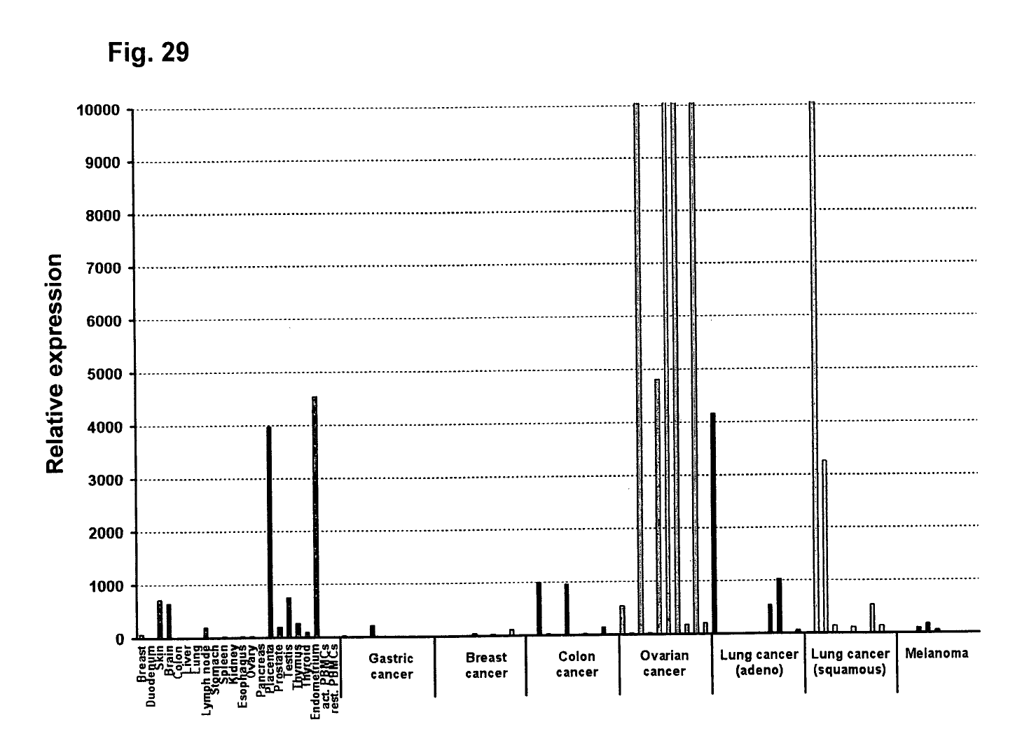

The nucleotide sequence according to SEQ ID NO:634 was deduced from SEQ ID NO:346 and codes for a member of the homeobox class of transcription factors (SEQ ID NO:635). Expression of these proteins is spatially and temporally regulated during embryonic development. Expression of SEQ ID NO:634 in normal and cancerous tissues was quantified by real-time RT-PCR using sequence-specific oligos (SEQ ID NO:636, 637); see FIG. 29. SEQ ID NO:634 is highly expressed in placenta and endometrium. Compared to other normal tissues, SEQ ID NO:634 is overexpressed in ovarian cancer and lung cancer. Based on these expression results SEQ ID NO:634 and its expression products qualify as molecular markers and/or target candidates for targeted therapies, in particular for these particular tumor types.

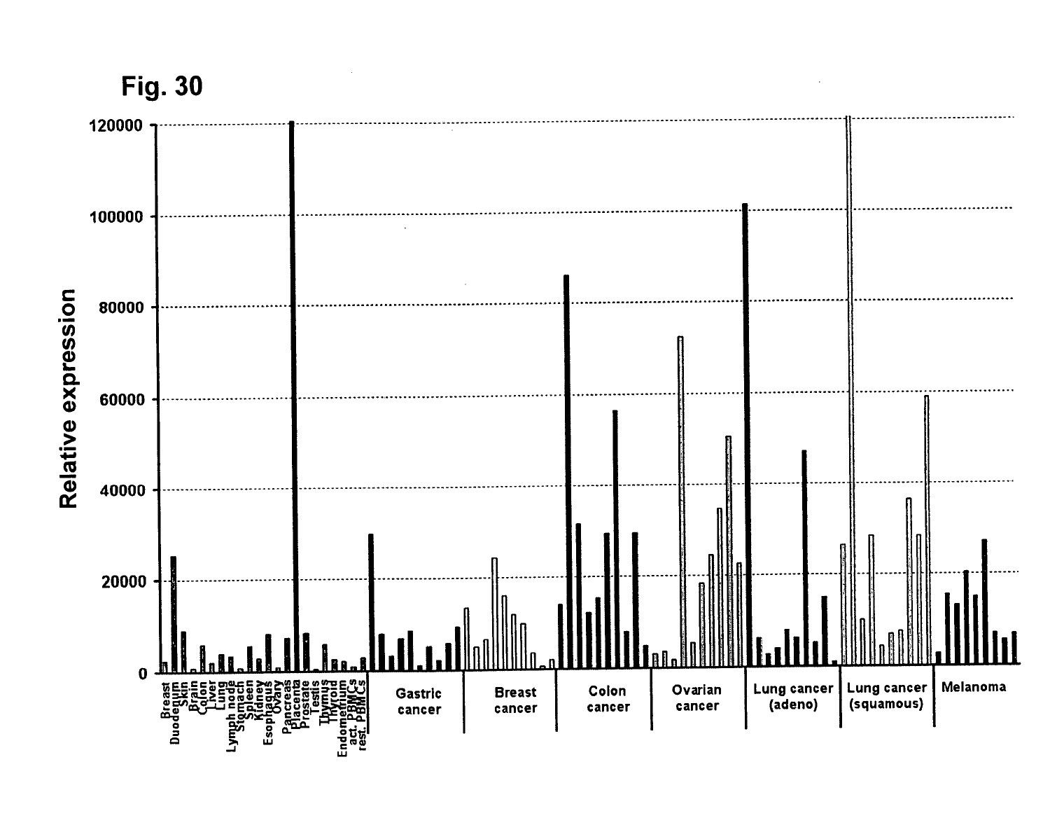

The nucleotide sequence according to SEQ ID NO:638 was deduced from SEQ ID NO:252 and codes for a member of the F-box protein family (SEQ ID NO:639) which is characterized by an approximately 40 amino acid motif, the F-box. The F-box proteins constitute one of the four subunits of ubiquitin protein ligase complex called SCFs (SKP1-cullin-F-box), which function in phosphorylation-dependent ubiquitination. The F-box proteins are divided into 3 classes: Fbws containing WD-40 domains, Fbls containing leucine-rich repeats, and Fbxs containing either different protein-protein interaction modules or no recognizable motifs. The protein encoded by this gene belongs to the Fbls class; in addition to an F-box, this protein contains 10 tandem leucine-rich repeats. This protein is an essential element of the cyclin A-CDK2 S-phase kinase. It specifically recognizes phosphorylated cyclin-dependent kinase inhibitor 1B (CDKN1B, also referred to as p27 or KIP1) predominantly in S phase and interacts with S-phase kinase-associated protein 1 (SKP1 or p19). Expression of SEQ ID NO:638 in normal and cancerous tissues was quantified by real-time RT-PCR using sequence-specific oligos (SEQ ID NO:640, 641); see FIG. 30. SEQ ID NO:638 is highly expressed in placenta. Compared to normal tissues, SEQ ID NO:638 is overexpressed in colon cancer, ovarian cancer, lung cancer, and malignant melanomas. Based on these expression results SEQ ID NO:638 and its expression products qualify as molecular markers and/or target candidates for targeted therapies, in particular for these particular tumor types.

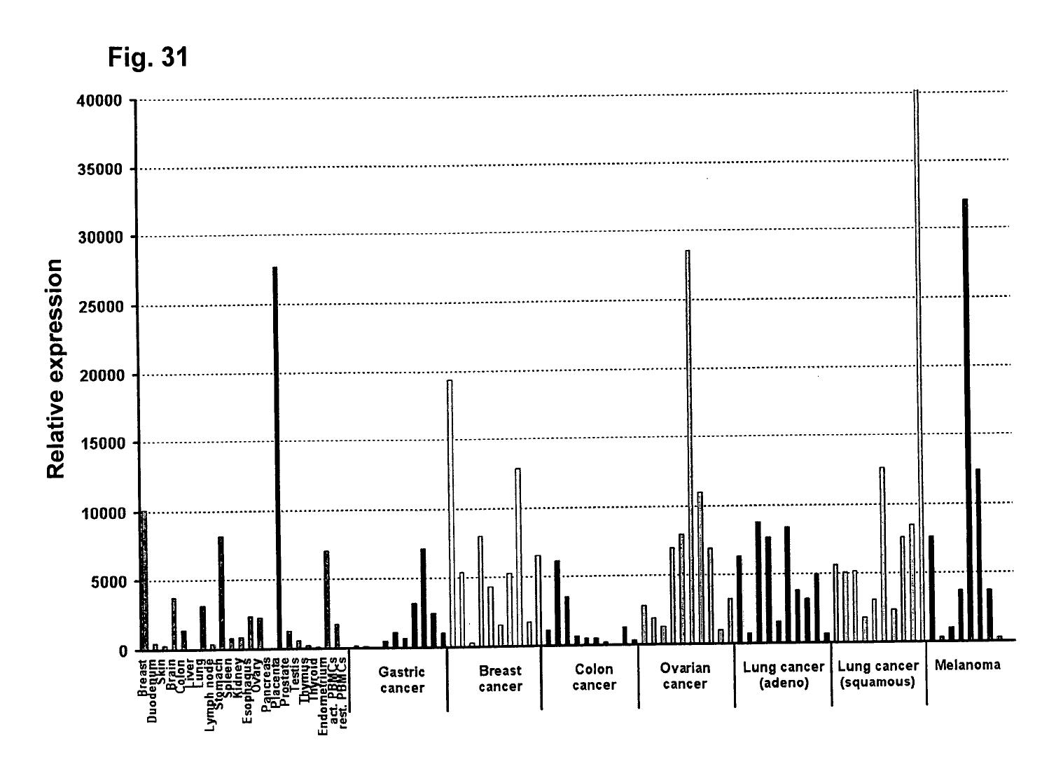

The nucleotide sequence according to SEQ ID NO:642 was deduced from SEQ ID NO:248 and codes for a 1.024 aa protein (SEQ ID NO:643) of unknown function. Expression of SEQ ID NO:642 in normal and cancerous tissues was quantified by real-time RT-PCR using sequence-specific oligos (SEQ ID NO:644, 645); see FIG. 31. SEQ ID NO:642 is highly expressed in placenta. Compared to normal tissues, SEQ ID NO:642 is overexpressed in breast cancer, ovarian cancer, lung cancer, and malignant melanomas. Based on these expression results SEQ ID NO:642 and its expression products qualify as molecular markers and/or target candidates for targeted therapies, in particular of these particular tumor types.

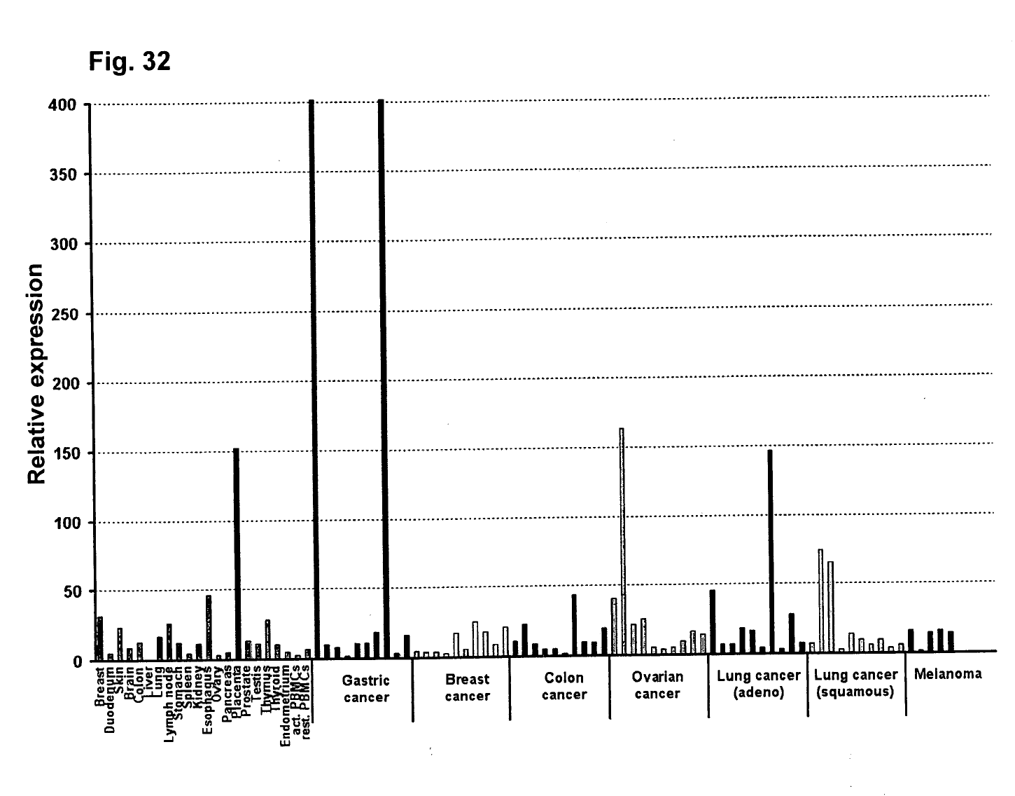

The nucleotide sequence according to SEQ ID NO:646 was deduced from SEQ ID NO:269. SEQ ID NO:646 represents a partial cDNA with no apparent open reading frame. Expression of SEQ ID NO:646 in normal and cancerous tissues was quantified by real-time RT-PCR using sequence-specific oligos (SEQ ID NO:647, 648); see FIG. 32. SEQ ID NO:646 is expressed in placenta. Compared to normal tissues, SEQ ID NO:646 is overexpressed in gastric cancer, ovarian cancer, and lung cancer. Based on these expression results SEQ ID NO:646 and its expression products qualify as molecular markers and/or target candidates for targeted therapies, in particular for these particular tumor types.

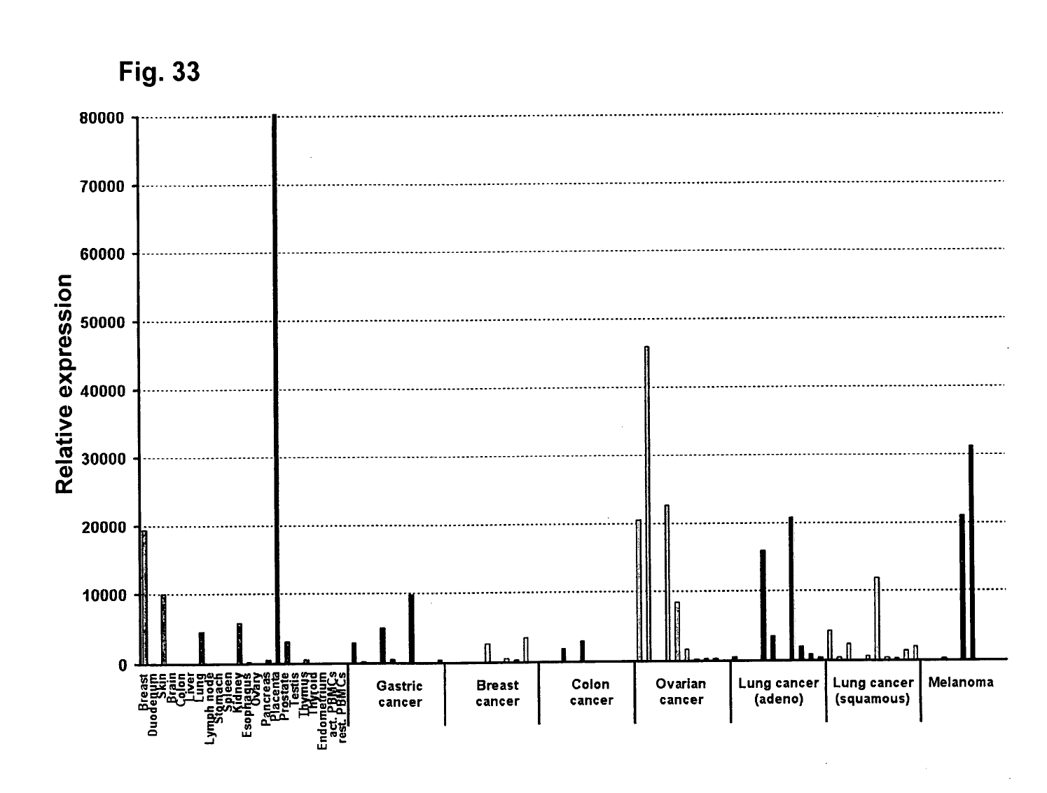

The nucleotide sequence according to SEQ ID NO:649 was deduced from SEQ ID NO:27 and codes for a 258 aa protein (SEQ ID NO:650) of unknown function. Expression of SEQ ID NO:649 in normal and cancerous tissues was quantified by real-time RT-PCR using sequence-specific oligos (SEQ ID NO:651, 652); cf. FIG. 33. SEQ ID NO:649 is highly expressed in placenta. Compared to normal tissues, SEQ ID NO:649 is overexpressed in ovarian cancer, lung cancer, and malignant melanomas. Based on these expression results SEQ ID NO:649 and its expression products qualify as molecular markers and/or target candidates for targeted therapies, in particular for these particular tumor types.

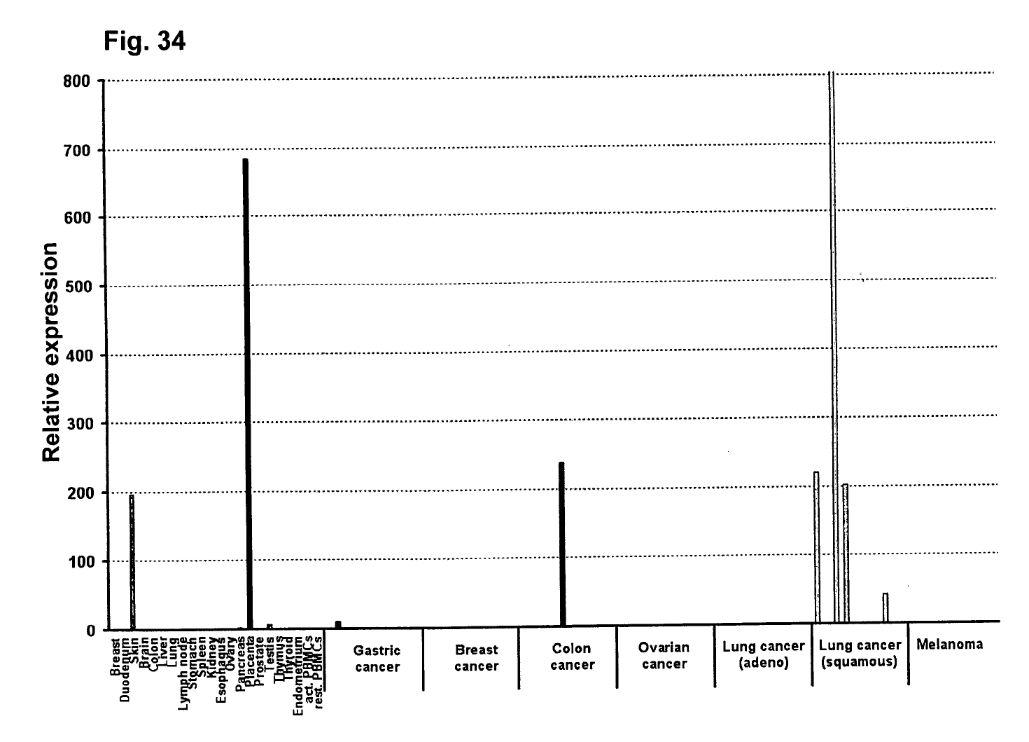

The nucleotide sequence according to SEQ ID NO:653 was deduced from SEQ ID NO:139. SEQ ID NO:653 represents a partial cDNA with no apparent open reading frame. Expression of SEQ ID NO:653 in normal and cancerous tissues was quantified by real-time RT-PCR using sequence-specific oligos (SEQ ID NO:654, 655); see FIG. 34. SEQ ID NO:653 is expressed in placenta and skin. Compared to other normal tissues, SEQ ID NO:653 is overexpressed in colon cancer, and lung cancer. Based on these expression results SEQ ID NO:653 and its expression products qualify as molecular markers and/or target candidates for targeted therapies, in particular for these particular tumor types.

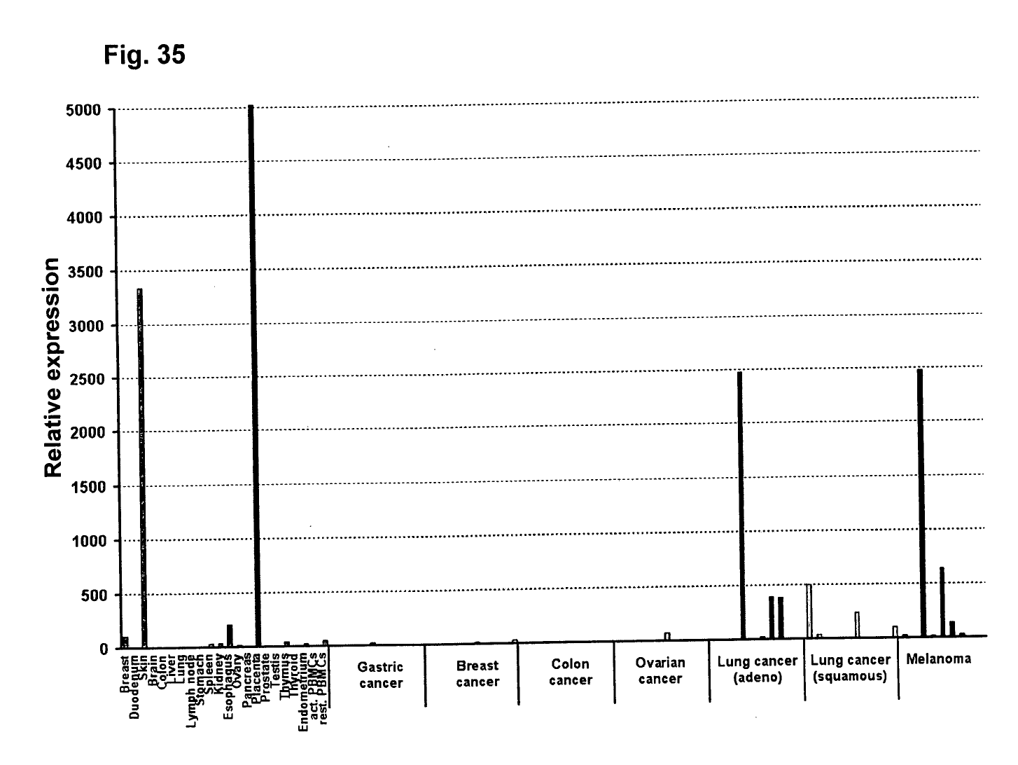

The nucleotide sequence according to SEQ ID NO:656 was deduced from SEQ ID NO:305 and codes for a 419 aa protein (SEQ ID NO:657) of unknown function. Expression of SEQ ID NO:656 in normal and cancerous tissues was quantified by real-time RT-PCR using sequence-specific oligos (SEQ ID NO:658, 659); see FIG. 35. SEQ ID NO:656 is expressed in placenta and skin. Compared to other normal tissues, SEQ ID NO:656 is overexpressed in lung cancer, and malignant melanomas. Based on these expression results SEQ ID NO:656 and its expression products qualify as molecular markers and/or target candidates for targeted therapies, in particular for these particular tumor types.

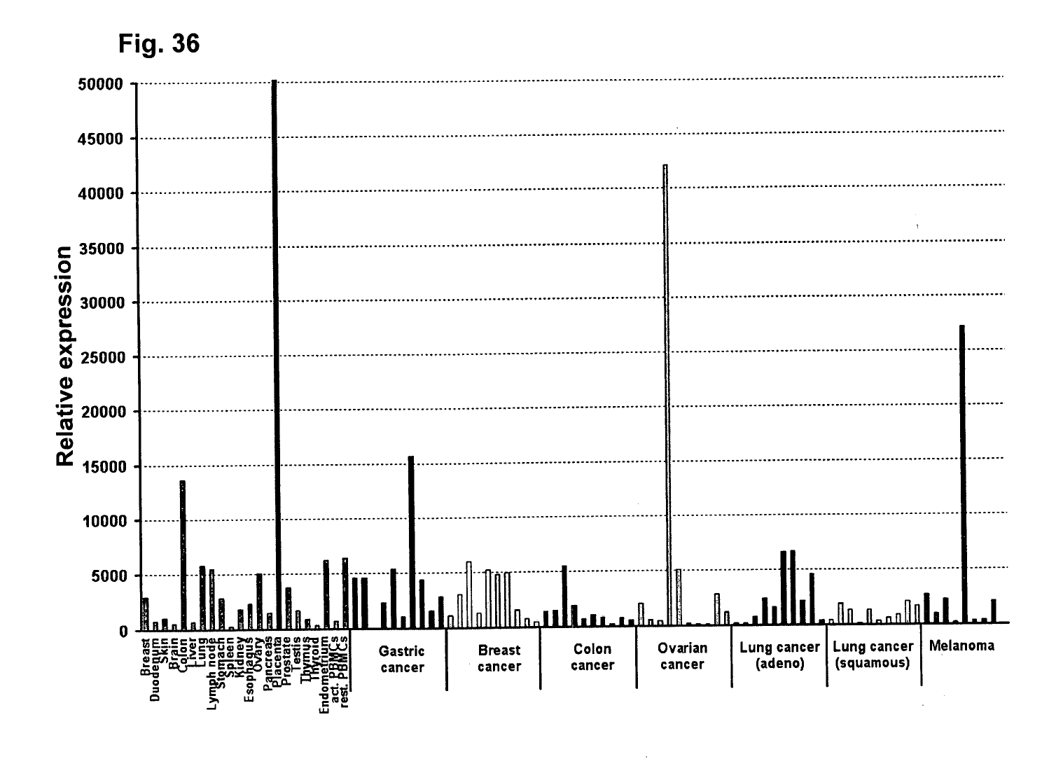

The nucleotide sequence according to SEQ ID NO:660 was deduced from SEQ ID NO:125 and codes for a a disintegrin and metalloproteinase with thrombospondin motif (SEQ ID NO:661). Expression of SEQ ID NO:660 in normal and cancerous tissues was quantified by real-time RT-PCR using sequence-specific oligos (SEQ ID NO:662, 663); see FIG. 36. SEQ ID NO:660 is highly expressed in placenta. Compared to normal tissues, SEQ ID NO:660 is overexpressed in gastric cancer, ovarian cancer, and malignant melanomas. Based on these expression results SEQ ID NO:660 and its expression products qualify as molecular markers and/or target candidates for targeted therapies, in particular for these particular tumor types.

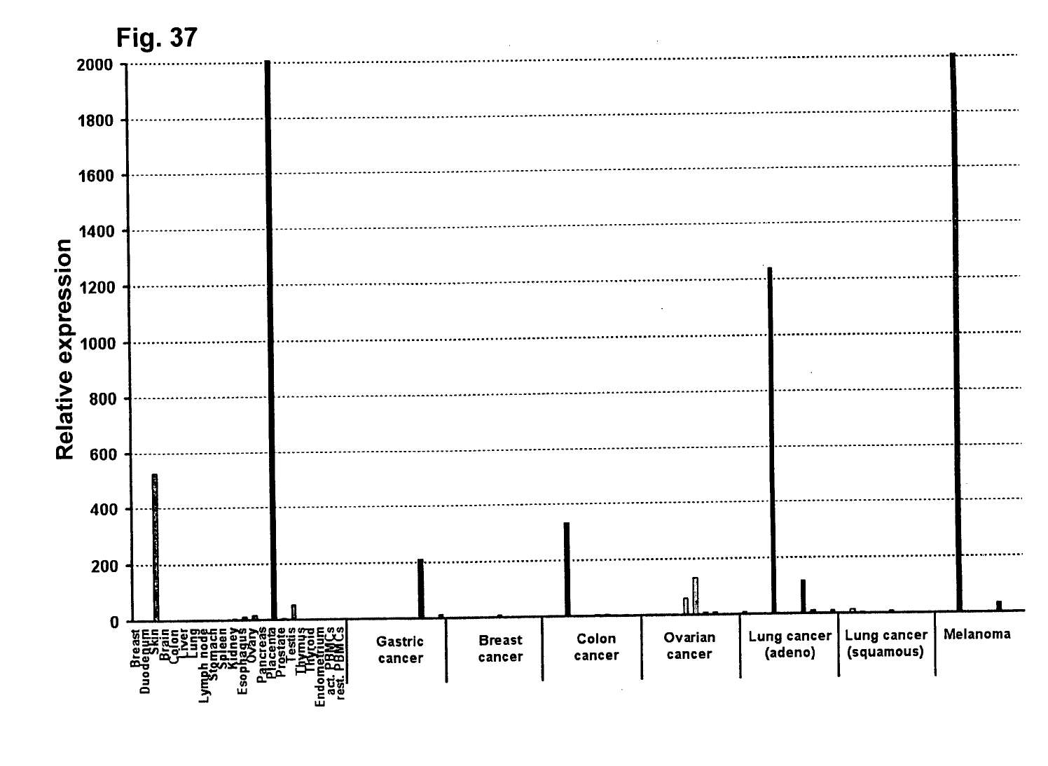

The nucleotide sequence according to SEQ ID NO:664 was deduced from SEQ ID NO:61 and codes for a 334 aa protein (SEQ ID NO:665) of unknown function. Expression of SEQ ID NO:664 in normal and cancerous tissues was quantified by real-time RT-PCR using sequence-specific oligos (SEQ ID NO:666, 667); see FIG. 37. SEQ ID NO:664 is expressed in placenta and skin. Compared to other normal tissues, SEQ ID NO:664 is overexpressed in gastric cancer, colon cancer, lung cancer, and malignant melanomas. Based on these expression results SEQ ID NO:664 and its expression products qualify as molecular markers and/or target candidates for targeted therapies, in particular for these particular tumor types.

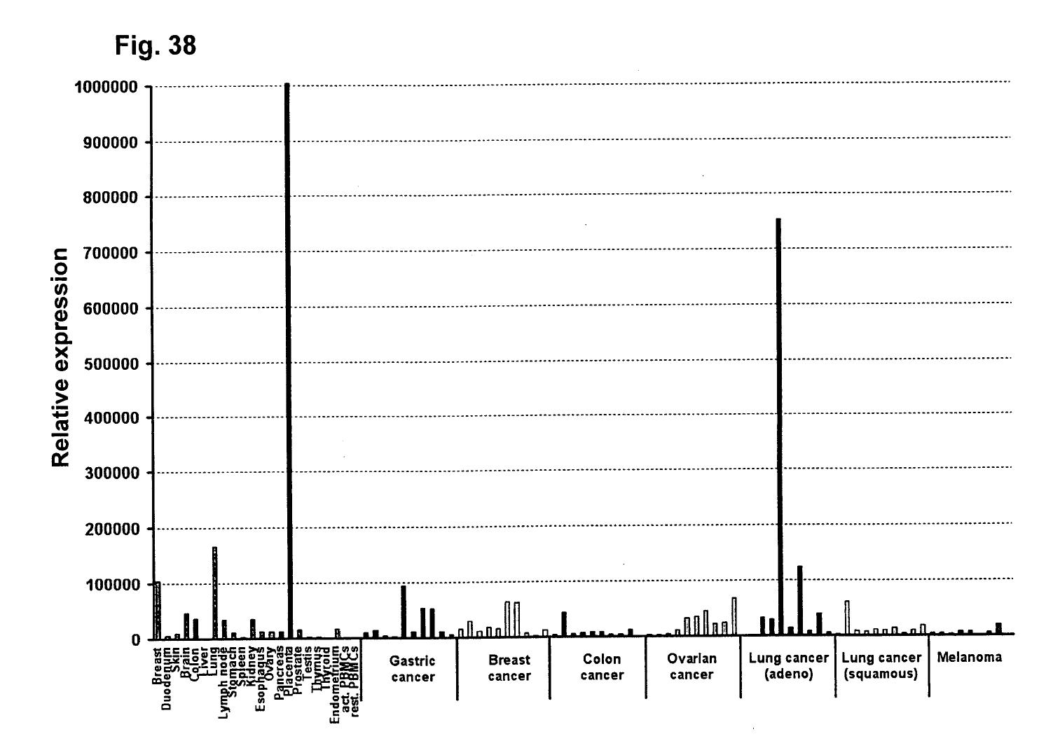

The nucleotide sequence according to SEQ ID NO:668 was deduced from SEQ ID NO:13. SEQ ID NO:668 represents a partial cDNA with no apparent open reading frame. Expression of SEQ ID NO:668 in normal and cancerous tissues was quantified by real-time RT-PCR using sequence-specific oligos (SEQ ID NO:669, 670); see FIG. 38. SEQ ID NO:668 is highly expressed in placenta. Compared to other normal tissues, SEQ ID NO:668 is overexpressed in lung cancer. Based on these expression results SEQ ID NO:668 and its expression products qualify as molecular markers and/or target candidates for targeted therapies, in particular for this particular tumor type.

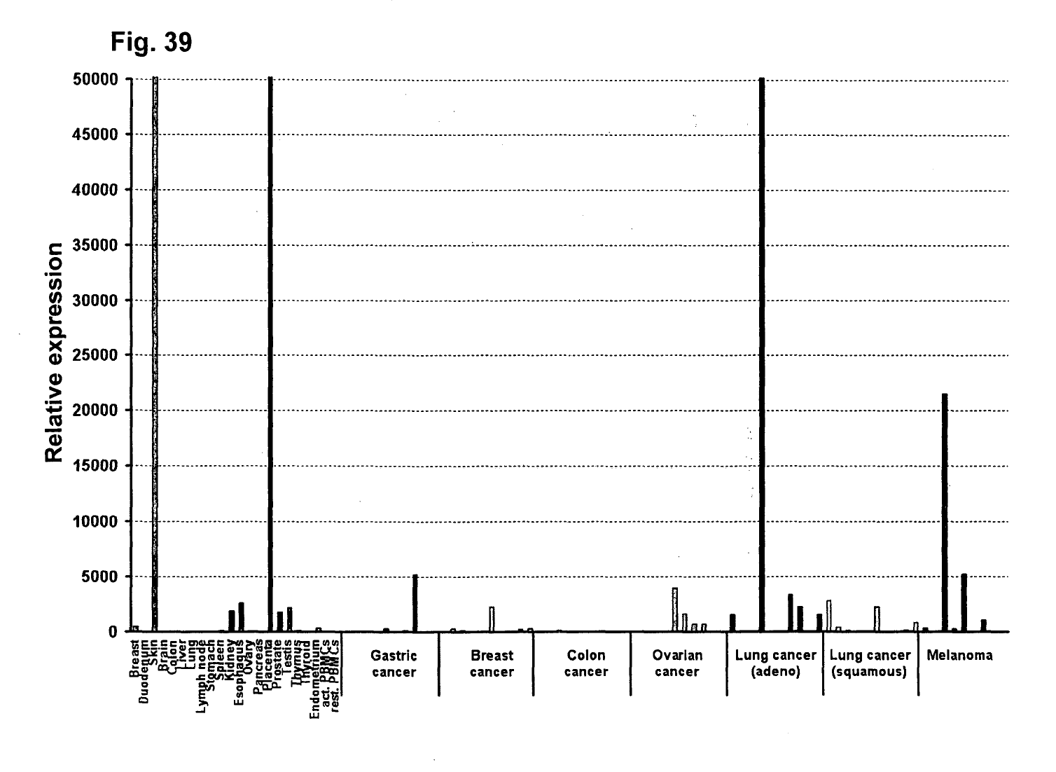

The nucleotide sequence according to SEQ ID NO:671 was deduced from SEQ ID NO:42 and codes for a 426 aa protein (SEQ ID NO:672) of unknown function. Expression of SEQ ID NO:671 in normal and cancerous tissues was quantified by real-time RT-PCR using sequence-specific oligos (SEQ ID NO:673, 674); see FIG. 39. SEQ ID NO:671 is expressed in placenta and skin. Compared to other normal tissues, SEQ ID NO:671 is overexpressed in lung cancer, and malignant melanomas. Based on these expression results SEQ ID NO:671 and its expression products qualify as molecular markers and/or target candidates for targeted therapies, in particular for these particular tumor types.

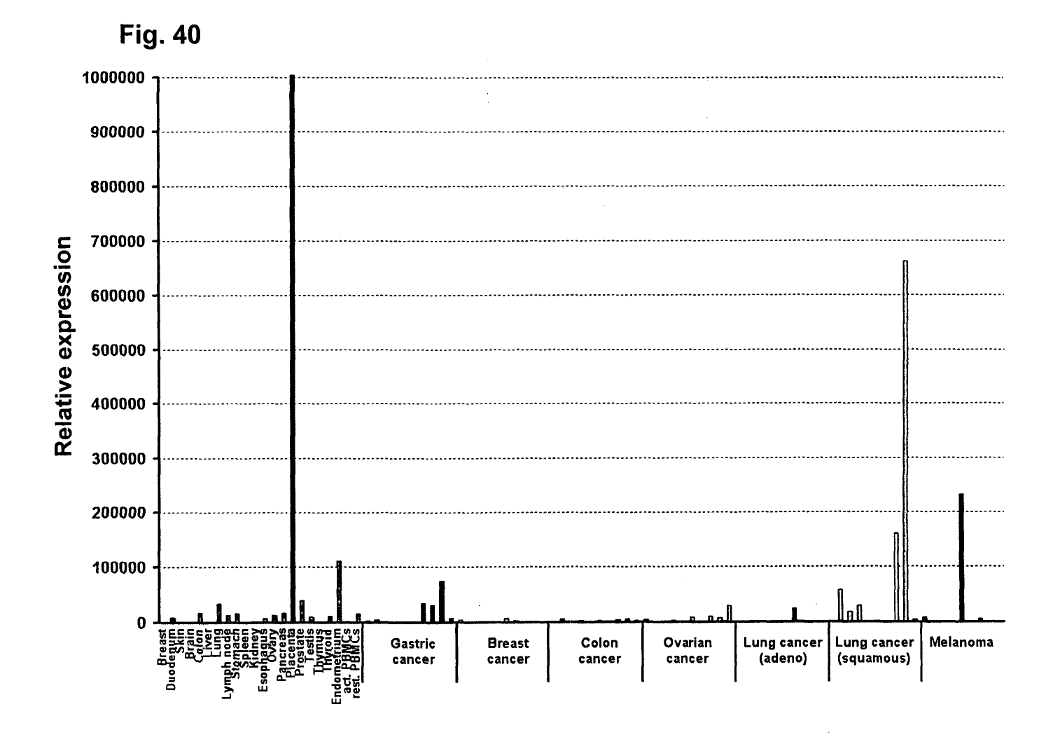

The nucleotide sequence according to SEQ ID NO:675 was deduced from SEQ ID NO:314 and codes for a 2.912 aa protein (SEQ ID NO:676). Expression of SEQ ID NO:675 in normal and cancerous tissues was quantified by real-time RT-PCR using sequence-specific oligos (SEQ ID NO:677, 678); see FIG. 40. SEQ ID NO:675 is highly expressed in placenta. Compared to other normal tissues, SEQ ID NO:675 is overexpressed in lung cancer, and malignant melanomas. Based on these expression results SEQ ID NO:675 and its expression products qualify as molecular markers and/or target candidates for targeted therapies, in particular for these particular tumor types.

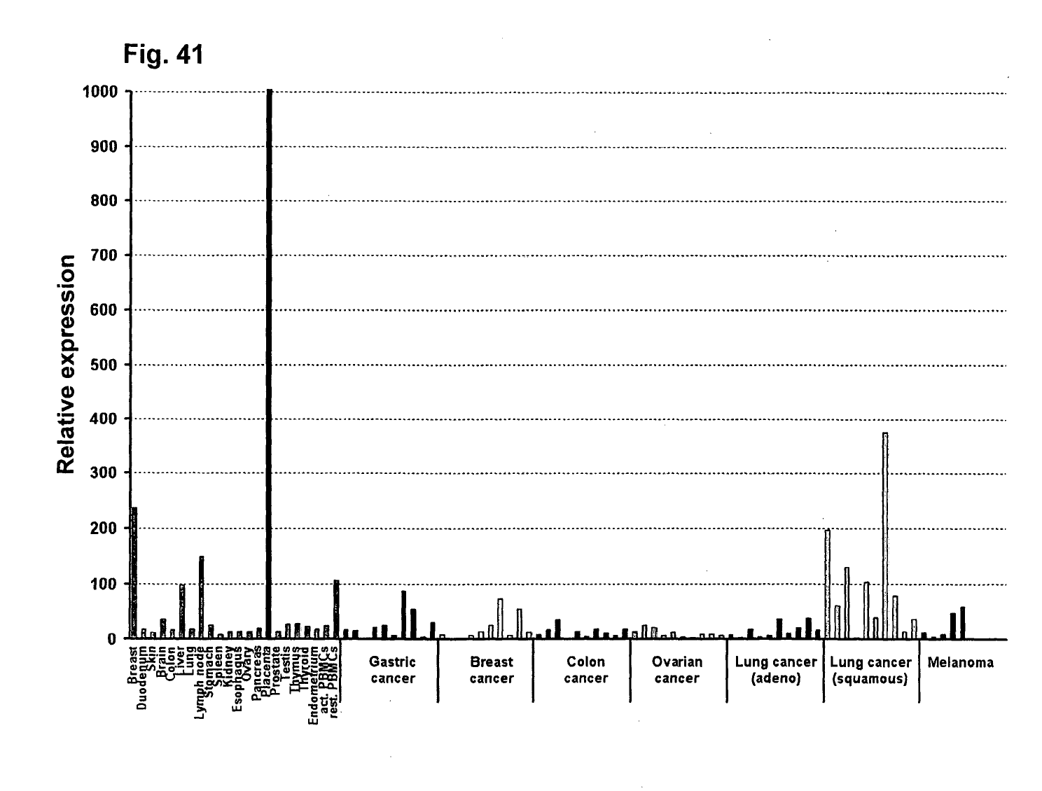

The nucleotide sequence according to SEQ ID NO:679 was deduced from SEQ ID NO:31. SEQ ID NO:679 represents a partial cDNA with no apparent open reading frame. Expression of SEQ ID NO:679 in normal and cancerous tissues was quantified by real-time RT-PCR using sequence-specific oligos (SEQ ID NO:680, 681); see FIG. 41. SEQ ID NO:679 is highly expressed in placenta. Compared to other normal tissues, SEQ ID NO:679 is overexpressed in lung cancer. Based on these expression results SEQ ID NO:679 and its expression products qualify as molecular markers and/or target candidates for targeted therapies, in particular for this particular tumor type.

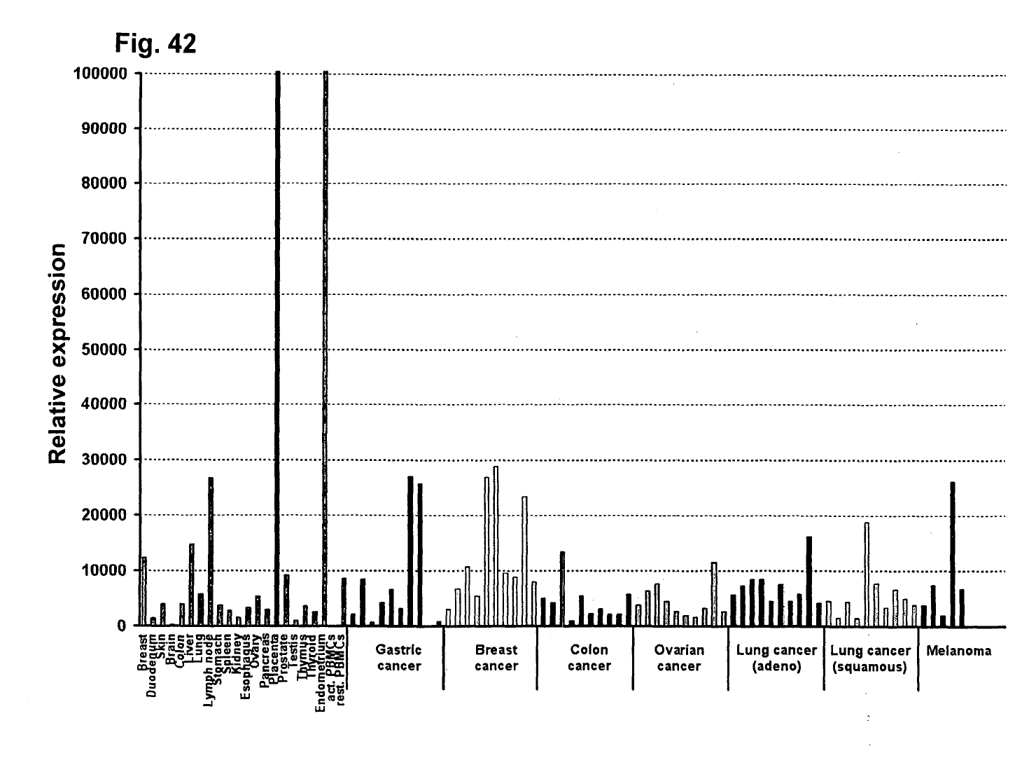

The nucleotide sequence according to SEQ ID NO:682 was deduced from SEQ ID NO:21 and codes for a 278 aa protein (SEQ ID NO:683). Expression of SEQ ID NO:682 in normal and cancerous tissues was quantified by real-time RT-PCR using sequence-specific oligos (SEQ ID NO:684, 685); see FIG. 42. SEQ ID NO:682 is highly expressed in placenta, endometrium, and at lower levels in lymph node. Compared to other normal tissues SEQ ID NO:682 is overexpressed in gastric cancer, breast cancer, and malignant melanomas. Based on these expression results SEQ ID NO:682 and its expression products qualify as molecular markers and/or target candidates for targeted therapies, in particular for these particular tumor types.

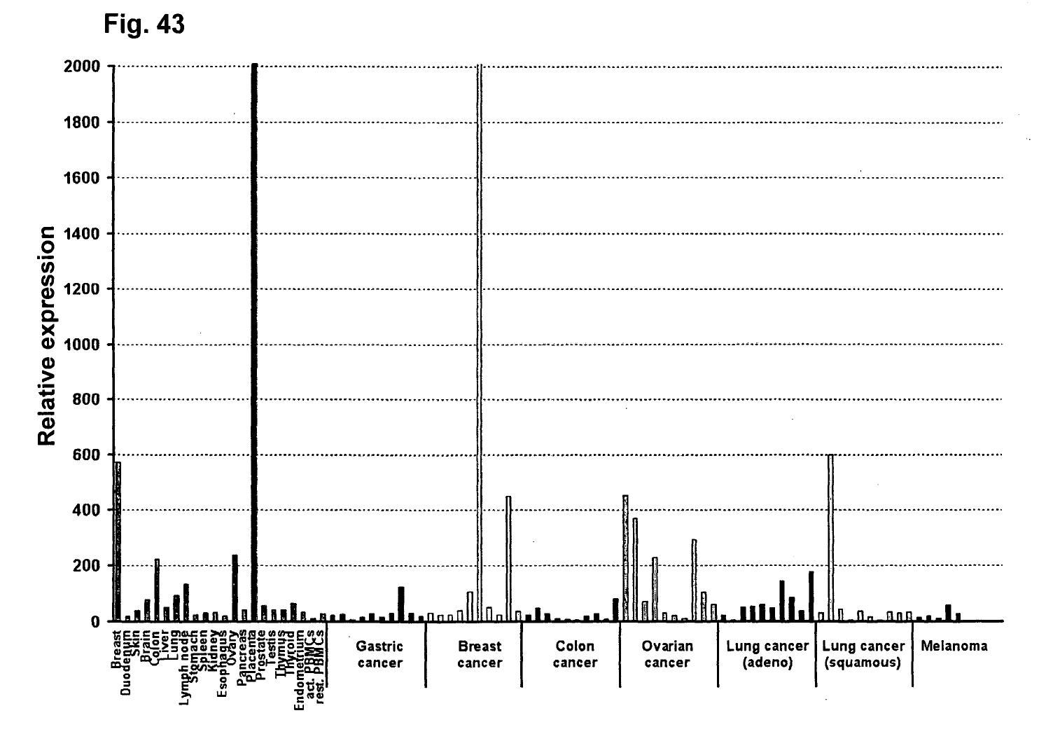

The nucleotide sequence according to SEQ ID NO:686 was deduced from SEQ ID NO:58. SEQ ID NO:686 represents a partial cDNA with no apparent open reading frame. Expression of SEQ ID NO:686 in normal and cancerous tissues was quantified by real-time RT-PCR using sequence-specific oligos (SEQ ID NO:687, 688); see FIG. 43. SEQ ID NO:686 is highly expressed in placenta and at lower levels in breast. Compared to other normal tissues, SEQ ID NO:686 is overexpressed in breast cancer, ovarian cancer, and lung cancer. Based on these expression results SEQ ID NO:686 and its expression products qualify as molecular markers and/or target candidates for targeted therapies, in particular for these particular tumor types.

1. A pharmaceutical composition, comprising an agent which
(I) inhibits expression or activity of a tumor-associated antigen and/or
(II) has tumor-inhibiting activity, and is selective for cells expressing or abnormally expressing a nucleic acid coding for a tumor-associated antigen or a tumor-associated antigen and/or
(III) when administered, selectively increases the amount of complexes between an MHC molecule and a tumor-associated antigen or a part thereof,
said tumor-associated antigen having a sequence encoded by a nucleic acid which is selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

2. The pharmaceutical composition as claimed in claim 1, in which the agent under (I) or (II) is an antisense nucleic acid which hybridizes selectively with the nucleic acid coding for the tumor-associated antigen or is an antibody which binds selectively to the tumor-associated antigen.

3. The pharmaceutical composition as claimed in claim 1, in which the agent comprises one or more components selected from the group consisting of:
(i) the tumor-associated antigen or a part thereof,
(ii) a nucleic acid which codes for the tumor-associated antigen or a part thereof,
(iii) an antibody which binds to the tumor-associated antigen or a part thereof,
(iv) an antisense nucleic acid which hybridizes specifically with a nucleic acid coding for the tumor-associated antigen,
(v) an siRNA directed against a nucleic acid coding for the tumor-associated antigen,
(vi) a host cell which expresses the tumor-associated antigen or a part thereof, and
(vii) isolated complexes between the tumor-associated antigen or a part thereof and an MHC molecule.

4. A pharmaceutical composition, comprising one or more components selected from the group consisting of:
(i) a tumor-associated antigen or a part thereof,
(ii) a nucleic acid which codes for a tumor-associated antigen or a part thereof,
(iii) an antibody which binds to a tumor-associated antigen or a part thereof,
(iv) an antisense nucleic acid which hybridizes specifically with a nucleic acid coding for a tumor-associated antigen,
(v) an siRNA directed against a nucleic acid coding for a tumor-associated antigen,
(vi) a host cell which expresses a tumor-associated antigen or a part thereof, and
(vii) isolated complexes between a tumor-associated antigen or a part thereof and an MHC molecule,
said tumor-associated antigen having a sequence encoded by a nucleic acid which is selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

5. The pharmaceutical composition as claimed in claim 3, in which the host cell additionally expresses an MHC molecule which binds to the tumor-associated antigen or the part thereof, wherein the host cell preferably is an antigen-presenting cell.

6. The pharmaceutical composition as claimed claim 2, in which the antibody is a monoclonal, chimeric, human or humanized antibody, or is a fragment of an antibody.

7. The pharmaceutical composition as claimed in claim 2, in which the antibody is coupled to a therapeutic or diagnostic agent.

8. The pharmaceutical composition as claimed in claim 1, which may be used for the treatment or prevention of cancer.

9. The pharmaceutical composition as claimed in claim 8, in which the cancer is a lung tumor, a breast tumor, a prostate tumor, a melanoma, a colon tumor, a gastric tumor, a pancreatic tumor, an ENT tumor, an ovarian tumor, a colorectal tumor, a cervical carcinoma, a colon carcinoma or a mammary carcinoma.

10. The pharmaceutical composition as claimed in claim 1, which is in the form of a vaccine and preferably is for therapeutic and/or prophylactic use.

11. A method of diagnosing or monitoring a cancer disease, which method comprises detecting or determining the quantity
(i) of a tumor-associated nucleic acid or of a part thereof, and/or
(ii) of a tumor-associated antigen or of a part thereof, and/or
(iii) of an antibody to the tumor-associated antigen or a part thereof and/or
(iv) of T lymphocytes which are specific to the tumor-associated antigen or to a part thereof
in a biological sample isolated from a patient,
said tumor-associated nucleic acid being selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c), and
said tumor-associated antigen having a sequence encoded by a nucleic acid which is selected from said group of nucleic acids.

12. The method as claimed in claim 11, in which the detection or determination of the quantity comprises
(i) contacting the biological sample with an agent which binds specifically to the tumor-associated nucleic acid or to the part thereof, to the tumor-associated antigen or the part thereof, to the antibody or to the T lymphocytes, and
(ii) detecting the formation of or determining the quantity of a complex between the agent and the nucleic acid or the part thereof, the tumor-associated antigen or the part thereof, the antibody or the T lymphocytes.

13. The method as claimed in claim 12, in which the agent which binds specifically to the tumor-associated nucleic acid or to the part thereof is an oligonucleotide or polynucleotide, which hybridizes specifically to said nucleic acid or to said part thereof.

14. The method as claimed in claim 12, in which the agent which binds specifically to the tumor-associated antigen or the part thereof is an antibody binding specifically to said tumor-associated antigen or to said part thereof.

15. The method as claimed in claim 12, in which the agent which binds specifically to the antibody is a protein or peptide binding specifically to said antibody.

16. The method as claimed in claim 12, in which the agent which binds specifically to the T lymphocytes is a cell presenting the complex between the tumor-associated antigen or the part thereof and an MHC molecule.

17. The method as claimed in claim 11 wherein said monitoring of said disease comprises determining regression, course or onset of said disease in a sample from a patient who has said disease or is suspected of falling ill with said disease.

18. The method as claimed in claim 12, in which the agent is labeled in a detectable manner.

19. The method as claimed in claim 11, in which the sample comprises body fluid and/or body tissue.

20. The method as claimed in claim 11, in which said cancer disease is characterized by expression or abnormal expression of said tumor-associated nucleic acid and preferably is further characterized by expression or abnormal expression of a tumor-associated antigen encoded by said tumor-associated nucleic acid.

21. A method of treating or preventing a disease characterized by expression or abnormal expression of a tumor-associated antigen, which method comprises administration of a pharmaceutical composition as claimed in claim 1,
said tumor-associated antigen having a sequence encoded by a nucleic acid which is selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

22. A method of treating, preventing, diagnosing or monitoring a disease characterized by expression or abnormal expression of a tumor-associated antigen, which method comprises administering an antibody binding to said tumor-associated antigen or to a part thereof and coupled to a therapeutic or diagnostic agent,
said tumor-associated antigen having a sequence encoded by a nucleic acid which is selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

23. The method as claimed in claim 22, in which the antibody is a monoclonal, chimeric, human or humanized antibody, or is a fragment of an antibody.

24. A method of treating a patient having a disease characterized by expression or abnormal expression of a tumor-associated antigen, which method comprises:
(i) providing a sample containing immunoreactive cells,
(ii) contacting said sample with a host cell expressing said tumor-associated antigen or a part thereof, under conditions which favor production of cytolytic or cytokine-releasing T cells against said tumor-associated antigen or said part thereof, and
(iii) introducing the cytolytic or cytokine-releasing T cells into the patient in an amount suitable for lysing cells expressing the tumor-associated antigen or a part thereof,
said tumor-associated antigen having a sequence encoded by a nucleic acid which is selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

25. The method as claimed in claim 24, in which the host cell recombinantly expresses an MHC molecule binding to the tumor-associated antigen or to a part thereof or endogenously expresses an MHC molecule binding to the tumor-associated antigen or to a part thereof.

26. A method of inhibiting the development of cancer in a patient, which method comprises administering an effective amount of a pharmaceutical composition as claimed in claim 1.

27. An agent, which binds specifically to a protein or polypeptide or to a part thereof, said protein or polypeptide being encoded by a nucleic acid selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

28. The agent as claimed in claim 27, which is an antibody, preferably a monoclonal, chimeric, human or humanized antibody, or a fragment of an antibody.

29. An antibody, which binds selectively to a complex of:
(i) a protein or polypeptide or a part thereof and
(ii) an MHC molecule to which said protein or polypeptide or said part thereof binds,
with said antibody not binding to (i) or (ii) alone and said protein or polypeptide being encoded by a nucleic acid selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c).

30. The antibody as claimed in claim 29, which is a monoclonal, chimeric or humanized antibody, or is a fragment of an antibody.

31. A conjugate between an agent as claimed in claim 27 and a therapeutic or diagnostic agent.

32. A kit for detecting cancer, which kit comprises agents for detecting or determining the quantity
(i) of a tumor-associated nucleic acid or of a part thereof, and/or
(ii) of a tumor-associated antigen or of a part thereof, and/or
(iii) of antibodies which bind to the tumor-associated antigen or to a part thereof, and/or
(iv) of T cells which are specific for a complex between the tumor-associated antigen or a part thereof and an MHC molecule,
said tumor-associated nucleic acid being selected from the group consisting of:
(a) a nucleic acid which comprises a nucleic acid sequence selected from the group consisting of SEQ ID NOs: 634, 1-540, 541, 545, 549, 553, 557, 560, 563, 566, 570, 574, 577, 580, 583, 587, 591, 595, 599, 602, 606, 610, 613, 617, 620, 624, 638, 642, 646, 649, 653, 656, 660, 664, 668, 671, 675, 679, 682, and 686, a part or derivative thereof,
(b) a nucleic acid which hybridizes with the nucleic acid of (a) under stringent conditions,
(c) a nucleic acid which is degenerate with respect to the nucleic acid of (a) or (b), and
(d) a nucleic acid which is complementary to the nucleic acid of (a), (b) or (c), and
said tumor-associated antigen having a sequence encoded by a nucleic acid which is selected from said group of nucleic acids.

33. The pharmaceutical composition as claimed in claim 1, in which the tumor-associated antigen comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.

34. The agent as claimed in claim 27 in which the protein or polypeptide comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.

35. A conjugate between an antibody as claimed in claim 29 and a therapeutic or diagnostic agent.

36. The method as claimed in claim 11, 22, or 24, in which the tumor-associated antigen comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.

37. The kit as claimed in claim 32, in which the tumor-associated antigen comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.

38. The antibody as claimed in claim 29, in which the protein or polypeptide comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.

39. The conjugate as claimed in claim 31, in which the protein or polypeptide comprises an amino acid sequence selected from the group consisting of SEQ ID NOs: 635, 542, 546, 550, 554, 567, 571, 584, 588, 592, 596, 603, 607, 614, 621, 625, 639, 643, 650, 657, 661, 665, 672, 676, and 683, a part or derivative thereof.
